(19) United States
(12) Patent Application Publication
(10) Pub. No.: US 2025/0066777 A1
(43) Pub. Date: Feb. 27, 2025

(54) METHODS AND AGENTS FOR INCREASING RBM3 EXPRESSION

(71) Applicant: FREIE UNIVERSITÄT BERLIN, Berlin (DE)

(72) Inventors: Marco PREUßNER, Berlin (DE); Florian HEYD, Berlin (DE)

(21) Appl. No.: 18/721,597

(22) PCT Filed: Dec. 19, 2022
(86) PCT No.: PCT/EP2022/086740

§ 371 Date: Jun. 18, 2024

(30) Foreign Application Priority Data
Dec. 20, 2021 (GB) 2118495.7
Oct. 24, 2022 (GB) 2215713.5

(51) Int. Cl. C12N 15/113; A61P 25/28; C12N 9/22; C12N 15/11; C12N 15/86; G01N 33/50

(52) U.S. Cl. CPC C12N 15/113; A61P 25/28; C12N 9/22; C12N 15/111; C12N 15/86; G01N 33/5023; C12N 2310/11; C12N 2310/20; C12N 2310/315; C12N 2310/321; C12N 2310/3231; C12N 2320/33; C12N 2750/14143; G01N 2500/10

(57) ABSTRACT

The invention provides agents (e.g., antisense oligonucleotides (ASOs), a CRISPR/Cas-based base editing system) capable of increasing expression of RNA-binding motif protein 3 (RBM3) by targeting a poison exon of RBM3, exon 3a, or a splice site thereof. Also disclosed are methods for increasing expression of RBM3 in a cell, methods of treating or preventing a disease affected by RMB3 expression in a subject, or methods for providing neuroprotective treatment to a subject.

This application claims priority from GB 2118495.7 filed 20 Dec. 2021 and GB 2215713.5 filed 24 Oct. 2022, the contents and elements of which are herein incorporated by reference for all purposes.

### FIELD OF THE INVENTION

The present invention relates generally to methods and materials for increasing expression of RNA-binding motif protein 3 (RBM3) and agents for that purpose, for example for use in therapy and prevention of disease.

### BACKGROUND

In hibernation and hypothermia, global protein synthesis and cell metabolism are downregulated, but low temperature also induces the expression of a small subset of proteins known as cold-shock proteins (Peretti, D. et al. Nature 518, 236-239 (2015)). Amongst these, RNA-binding motif protein 3 (RBM3) and cold-inducible RNA binding protein (CIRP, also known as CIRBP) are cold-shock proteins expressed at high levels in the brain^{1,2,30,31}. RBM3 and CIRPB (and other RNA-binding proteins, RBPs) regulate splicing, stability and transport of mRNA and are critical regulators of gene expression (GE).

Despite the evolutionary conservation³ and extreme temperature sensitivity⁴ of RBM3 and CIRPB, the mechanistic basis for cold induced transcript expression remained enigmatic for a long time. A global analysis of gene expression indicated temperature-regulated alternative splicing (AS) coupled to nonsense-mediated decay (NMD) as a global mechanism of controlling temperature-dependent gene expression⁵.

Alternative splicing of pre-mRNA leads to different mRNA isoforms. The NMD pathway recognizes mRNA isoforms encoding premature termination codons (PTCs) and targets these mRNAs for degradation, thus allowing splicing-controlled regulation of gene expression levels⁶. NMD-inducing isoforms are frequently found in RNA binding proteins⁵ and a heat-induced PTC-containing isoform of CIRBP provides an explanation for cold-induced CIRBP expression⁷. In a global analysis, temperature-controlled NMD isoforms of various RNA-binding proteins (RBPs) were identified along with a heat-induced NMD isoform of RBM3 (Neumann A., “Leveraging RNA-sequencing data to obtain insights about mRNA splicing: from daily rhythms to secretory adaptations and cryptic splice sites” thesis, Appendix I publication 6, 2019). Neumann et al.⁵ reports over 60 RBPs with temperature-regulated NMD exons. Preussner et al. (“Rhythmic gene expression is controlled by alternative splicing triggering nonsense mediated decay”, Abstract submitted at the 24^{th} Annual meeting of the RNA Society, 2019) discusses the aim of establishing methods to alter the expression levels of RBM3 and CIRBP by interfering with NMD-inducing splicing isoforms.

While CIRBP has diverse functions which range from circadian sleep homeostasis to inflammation and cancer⁸⁻¹¹, RBM3 is strongly associated with the neuroprotective effect of hypothermia¹². The neuroprotective function of RBM3 has been confirmed in independent systems which include hypoxic-ischemic brain injury¹³, exposure to neurotoxins¹⁴ and neurodegenerative diseases (e.g. prion-infected and Alzheimer-type mice)¹⁵. More specifically, RBM3 overexpression achieved either by inducing hypothermia or by lentiviral delivery results in synaptic protection in a mouse model of Alzheimer's disease and throughout the course of prion disease in mice, while preventing behavioural deficits and neuronal loss and significantly prolonging survival¹⁵.

Therapeutic hypothermia is extensively used in clinical practice for neuroprotection—including in neonatal hypoxic ischemic encephalopathy through to head injury, stroke and during cardiac surgery in adults.

The mechanisms of hypothermia-induced neuroprotection in humans are not fully understood Furthermore induced cooling in humans is not without risk, with high prevalence of blood clots, pneumonia, etc. and requires an intensive care set up.

The present invention has been devised in light of the above considerations.

### SUMMARY OF THE INVENTION

The present invention relates to increasing the levels of RBM3 without requiring induction of hypothermia. The inventors identified an exon, which was previously not experimentally characterised, that is included in an isoform of RBM3 mRNA at warm temperatures, and confirmed that this exon is responsible for non-sense mediated decay of RBM3 thus leading to reduced RBM3 expression at warm temperatures.

Agents targeting this exon or a splice site thereof increase expression of RBM3 without the need for cooling. A variety of such agents are described herein.

Peretti et al.¹⁵ describes the use of a lentiviral vector to induce expression of RBM3 in a mouse model of Alzheimer's disease and prion disease but this article is not concerned with the mechanism responsible for cold-induction of RBM3 and does not identify an alternative splicing isoform of RBM3.

Neumann (“Leveraging RNA-sequencing data to obtain insights about mRNA splicing: from daily rhythms to secretory adaptations and cryptic splice sites”, Appendix I publication 6, 2019) characterises NMD isoforms at different temperatures and identifies unannotated NMD variants of various RNA-binding proteins including RBM3. The NMD variant of RBM3 includes an NMD exon which is described only in a schematic which shows it being between exons 3 and 4 (Figure S2A). The inclusion of the NMD exon was not identified as a significant splicing event because the difference between its inclusion frequency (percent spliced in, PSI) at warm versus cold temperature was very low (dPSI<15%, see legend of Figure S2). Moreover, the inclusion level of the RBM3 NMD exon was only ˜3% PSI even at 38° C. (Figure S2B) which was the lowest reported in the global analysis, with the other RBP heat-included NMD exons having over 20% or even 60% PSI at 38° C. (hnRNP DL and CIRBP in FIG. 2D). Neumann does not disclose the effect of preventing the inclusion of this exon on the relative expression of RBM3 and does not disclose the specific sequence or position of this exon.

Neumann et al.⁵ does not identify an exon between exons 3 and 4 of RBM3. The alternative splicing events relating to RBM3 identified in Neumann et al.⁵ are alternative terminal exons, however these were not identified as temperature-dependent (marked as ‘undirected’, see sheet 3 of Table EV1).

Llorian et al.³² investigates alternative splicing of RBM3 among other genes in aortic smooth muscle cells during differentiation. It describes introns 3 and 4 of RBM3 as flanking a poison exon and show increased retention of the introns in differentiated cells. The authors do not investigate the effect of alternative splicing on the level of RBM3 mRNA, do not investigate the role of temperature on alternative splicing, and do not confirm or investigate the inclusion of the exon.

The present inventors identified that an NMD inducing exon between exons 3 and 4 surprisingly dominates the temperature-controlled expression of RBM3 despite its low inclusion level at high temperatures. A dramatic increase (3-4 fold) of RBM3 expression is achieved by agents capable of affecting alternative splicing of RBM3 pre-mRNA such as antisense oligonucleotides to prevent inclusion of this exon as described herein and demonstrated in the Examples.

In one aspect, the present invention provides a method for inhibiting non-sense mediated decay of RBM3-encoding mature mRNA in a cell, the method comprising exposing the cell to an agent, wherein the agent is capable of hybridising to a region of the pre-mRNA of RBM3 such as to alter the splicing of the pre-mRNA such that in the resulting mature mRNA exon 3a is not incorporated. The method may be in vitro or ex vivo. In some embodiments, the method is in vivo. The cell may be a mammalian cell e.g. a primate cell, optionally the cell may be a mouse cell or human cell. The cell is preferably a neuron, astrocyte, oligodendrocyte, microglial cell, ependymal cell or brain stem cell.

The term “exposing” as used herein refers to exposing a cell to the agent (e.g. ASO) or delivering the agent to the cell in such a manner or under such conditions that the agent enters the cell. See, for example Juliano et al., Bioconjug Chem. 2012 Feb. 15; 23(2): 147-57 (incorporated herein in its entirety). The agent (e.g. ASO) is delivered to the interior of the cell and enters the cell nucleus. The agent is exogenous to the cell. In one embodiment, the cell is contacted with a vector (e.g., a viral genome, a plasmid, an artificial chromosome) that enters the cell. In the cell, the vector causes expression of the ASO in the cell, e.g., from the cellular genome or from an exogenous nucleic acid. ASO can be introduced into cells in vivo or ex vivo.

“Capable of hybridising”—hybridisation assays are known in the art and generally involve using complementary nucleic acid probes (such as in situ hybridization using labelled probe, Northern blot and related techniques). In some embodiments, the hybridisation assay is an in situ hybridisation assay using a labelled probe, such as a fluorescently labelled probe.

Suitable selective hybridisation conditions for oligonucleotides of 17 to 30 bases include hybridization overnight at 42° C. in 6×SSC and washing in 6×SSC at a series of increasing temperatures from 42° C. to 65° C. One common formula for calculating the stringency conditions required to achieve hybridization between nucleic acid molecules of a specified sequence homology is (Sambrook et al., 1989): Tm=81.5° C.+16.6 Log [Na+]+0.41 (% G+C)−0.63 (% formamide)−600/#bp in duplex.

In another aspect, the invention provides a method for treating or preventing a disease affected by RMB3 expression in a subject, the method comprising administering to the subject an agent, wherein the agent is capable of hybridising to a region of the pre-mRNA of RBM3 such as to alter splicing of the pre-mRNA such that in the resulting mature mRNA exon 3a is not incorporated. In one aspect, the invention provides a method for increasing neuroprotection in a subject, the method comprising administering to the subject an agent, wherein the agent is capable of hybridising to a region of the pre-mRNA of RBM3 such as to alter splicing of the pre-mRNA such that in the resulting mature mRNA exon 3a is not incorporated.

Agents of the invention may be capable of preventing incorporation of all or part of exon 3a.

As used herein, “expression” may be gene expression or protein expression and thus may be measured by quantifying levels of a transcript of RBM3 or the protein levels of RBM3.

Exemplary methods for measuring expression of RBM3 are discussed in the “Methods” section hereinafter.

Methods and agents of the invention may be used to treat or prevent a disease such as: a neurological disease, and/or the disease is neonatal hypoxic ischemic encephalopathy, head injury, or stroke; anxiety or depression; a neurodegenerative disease optionally selected from Alzheimer's disease, Parkinson's disease, prion disease, frontotemporal dementia, a tauopathy, amyotrophic lateral sclerosis (ALS), and vascular dementia; or neurological damage, optionally caused during cardiac surgery or induced coma.

In some embodiments, the region of the pre-mRNA of RBM3 is selected from: a region within exon 3a, a region spanning a splice site of exon 3a, a region located within 250 nucleotides upstream exon 3a and a region located within 250 nucleotides downstream exon 3a. In some embodiments, the region is located within 200 nucleotides upstream exon 3a or within 200 nucleotides downstream exon 3a. The region may be within 10, 20, 30, 40, 50, 60, 70, 80, 90 or 100 nucleotides upstream exon 3a. The region may be within 10, 20, 30, 40, 50, 60, 70, 80, 90 or 100 nucleotides downstream exon 3a. The region may be within 110, 120, 130, 140, 150, 160, 170, 180, 190 or 200 nucleotides upstream exon 3a. The region may be within 110, 120, 130, 140, 150, 160, 170, 180, 190 or 200 nucleotides downstream exon 3a. The region may be within 210, 220, 230, 240 or 250 nucleotides upstream exon 3a. The region may be within 210, 220, 230, 240 or 250 nucleotides downstream exon 3a. The region may be upstream or downstream exon 3a and corresponding to annotated intron 3 of the RBM3 gene.

In some embodiments, the region comprises a splice enhancer element. For example, in some embodiments, the region corresponds to SEQ ID NO: 27, preferably SEQ ID NO: 29. In other embodiments, the region corresponds to SEQ ID NO: 15, 16, 17, 19, 21, 23, or 25.

In some embodiments, the region spans the 5′ splice site of exon 3a. In certain embodiments, the region comprises nucleotides 257 to 269, 258 to 269, 259 to 269, 260 to 269, 261 to 269, 262 to 269, 263 to 269, 264 to 269, 265 to 269, 266 to 269, or 267 to 269 of SEQ ID NO: 6 and/or nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33. In some embodiments, the region comprises nucleotides 259 to 271, 260 to 271, 261 to 271, 262 to 271, 263 to 271, 264 to 271, 265 to 271, 266 to 271, 267 to 271, 268 to 271, or 269 to 271 of SEQ ID NO: 5.

In some embodiments, the region spans the 3′ splice site of exon 3a. In some embodiments, the region comprises nucleotides 1 and 2, 1 to 3, 1 to 4, 1 to 5, 1 to 6, 1 to 7, 1 to 8, 1 to 9, or 1 to 10 of SEQ ID NO: 6 and/or nucleotides 136 to 147, 137 to 147, 138 to 147, 139 to 147, 140 to 147, 141 to 147, 142 to 147, 143 to 147, 144 to 147, or 145 to 147 of SEQ ID NO: 32. In certain embodiments, the region comprises nucleotides 1 and 2, 1 to 3, 1 to 4, 1 to 5, 1 to 6, 1 to 7, 1 to 8, 1 to 9, or 1 to 10 of SEQ ID NO: 5.

In certain embodiments, the agent is an antisense oligonucleotide (ASO). In this case, the method for inhibiting non-sense mediated decay of RBM3-encoding mature mRNA in a cell, the method for increasing neuroprotection and/or the method for treating or preventing a disease according to the invention may further comprise expressing the ASO from a transgene encoding the ASO which is introduced into or administered to said cell or subject. Preferably, the viral vector is a recombinant AAV vector.

In another aspect, the present invention provides an ASO as described herein. Also provided is an expression construct encoding the ASO according to the invention. Preferably the expression construct is a vector or a viral vector. Also provided is a host cell comprising the expression construct.

“Antisense oligonucleotide”—as used herein, the terms ‘antisense oligonucleotide’, ‘ASO’ and ‘antisense oligomer’ are used interchangeably and refer to a polynucleotide, comprising nucleotides, that hybridises to a target nucleic acid (e.g., pre-mRNA) sequence by Watson-Crick base pairing or wobble base pairing (G-U).

Preferably, the ASO is complementary or essentially complementary to all or part of the region of the pre-mRNA of RBM3 described herein.

The ASO may be of any length suitable for hybridising to the pre-mRNA and effective in altering splicing. In some embodiments, the ASO is 10 to 30 nucleotides long, preferably, the ASO is 25 nucleotides long, or the ASO is 15, 16, 17, 18 or 19 nucleotides long. In certain embodiments the ASO is 19 nucleotides long. In certain embodiments, the ASO is 8 to 300, preferably 18 to 30 nucleotides long, and more preferably, the ASO is 20, 21, 22, 23, 24 or 25 nucleotides long.

In specific embodiments, the ASO is 30 to 40, 41 to 50, or 51 to 300 nucleotides long. It will be appreciated that where a DNA sequence is specified herein, e.g. with reference to a Figure or SEQ. ID No, unless context requires otherwise, the “RNA equivalent”, with U substituted for T where it occurs, is disclosed mutatis mutandis.

“Complementary to”—for the purpose of the invention, the “complement of a nucleotide sequence represented in SEQ ID NO: X” is the nucleotide sequence which can be derived from the represented nucleotide sequence by replacing the nucleotides through their complementary nucleotide according to Chargaff's rules (A^{⇔}T or U; G^{⇔}C) and reading the sequence in the 5′ to 3′ direction, i.e. in opposite direction of the represented nucleotide sequence.

When polynucleotides (e.g., oligonucleotides, ASOs, mRNA, gRNA etc.) are “complementary” to one another, hybridisation occurs in an antiparallel configuration between two single-stranded polynucleotides.

“Essentially complementary to”—ASOs may have exact sequence complementarity to the target sequence or near complementarity (e.g., be essentially complementary, having sufficient complementarity to bind the target sequence and alter splicing of RBM3 pre-mRNA).

In either case the ASOs may block or inhibit the binding of spliceosomal complexes or spliceosomal components or a trans-regulatory element to the pre-mRNA; ASOs are designed so that they bind (hybridise) to a target nucleic acid (e.g., a pre-mRNA transcript) and remain hybridised under physiological conditions.

Typically, if they hybridise to a site other than the intended (target) nucleic acid sequence, they hybridise to a limited number of sequences that are not a target nucleic acid (to few sites other than a target nucleic acid). Design of an ASO can take into consideration the occurrence of the target nucleic acid sequence or a sufficiently similar nucleic acid sequence in other locations in the genome or cellular RNA/transcriptome, such that the likelihood the ASO will bind other sites and cause ‘off-target’ effects is limited.

An agent (e.g. ASO) need not hybridise to all nucleobases in a target sequence and the nucleobases to which it does hybridise may be contiguous or noncontiguous. ASOs may hybridise over one or more segments of a target nucleic acid, such that intervening or adjacent segments are not involved in the hybridization event (e.g., a loop structure or hairpin structure may be formed). In certain embodiments, an ASO hybridises to noncontiguous nucleobases in a target nucleic acid. For example, an ASO can hybridise to nucleobases in a target nucleic acid that are separated by one or more nucleobase(s) to which the ASO does not hybridise.

Thus an agent (e.g. ASO) may comprise a sequence of complementarity that is complementary with at least 8, at least 9, or at least 10 contiguous nucleotides (nt) of a sequence within a region between exons 3 and 4. In some embodiments, the sequence of complementarity is complementary with at least 8, at least 9 or at least 10 contiguous nucleotides of a sequence within exon 3a. In some embodiments, the sequence of complementarity is complementary with at least 8, at least 9 or at least 10 contiguous nucleotides that span a splice site of exon 3a. In some embodiments, the sequence of complementarity is complementary with at least 8, at least 9 or at least 10 contiguous nucleotides of a splice enhancer element.

“Complementarity” (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an oligomeric compound, e.g., an ASO or gRNA, need not be 100% complementary to that of its target nucleic acid to hybridise. In certain embodiments, “essentially complementary” ASOs can comprise at least about 70%, at least about 80%, at least about 90%, at least about 95%, or at least about 99% sequence complementarity to a target region within the target nucleic acid sequence to which they are targeted.

For example, an ASO in which 18 of 20 nucleobases of the oligomeric compound are complementary to a target region, and would therefore specifically hybridise, would represent 90 percent complementarity. In this example, the remaining noncomplementary nucleobases may be clustered together or interspersed with complementary nucleobases and need not be contiguous to each other or to complementary nucleobases. An ASO which is 18 nucleobases in length having 4 (four) noncomplementary nucleobases which are flanked by two regions of complete complementarity with the target nucleic acid would have 77.8% overall complementarity with the target nucleic acid and would thus fall within this scope

When considering ASOs which are capable of hybridising to the sense strand of a target region it will be appreciated that the longer the total length of the sense or antisense region, the less stringent the requirements of complementarity or sequence identity between these regions and the (complement of the) corresponding target region.

However, it is preferred that the nucleic acid of interest includes a sequence of about 19 consecutive nucleotides, particularly about 25 nucleotides, with 100% sequence identity to the corresponding part of the target nucleic acid.

“Corresponding to”—a nucleotide or a nucleotide sequence or region at a position in one sequence corresponds to a position of a nucleotide or a nucleotide sequence or region in a similar specific nucleotide sequence when they are optimally aligned. Typically, the corresponding regions will share a high degree of sequence identity.

“Optimal alignment”—the optimal alignment of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. on the World Wide Web at

https://www.ebi.ac.uk/Tools/emboss/using

default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Sequence identity”—for the purpose of this invention, the sequence identity of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues.

In some embodiments, the ASO comprises or consists of one of SEQ ID NOs: 40, 41, 42, 43, 44, 45, 46, 47, 48, 49 and 50, optionally having one, two or three substitutions. In some embodiments, the ASO comprises or consists of one of SEQ ID NOs: 51, 52, 53, 54, 55, 56 and 57, optionally having one, two or three substitutions. In certain embodiments, the ASO comprises or consists of one of SEQ ID NOs: 34, 35, 36, 37, 38, 39, 69 and 70 optionally having one, two or three substitutions.

In some embodiments, the ASO comprises or consists of one of SEQ ID NOs: 40, 41, 42, 43, 44, 46, 47, 48, 49, 50, 83, 84, 85, 86 and 87 optionally having one, two or three substitutions.

In some embodiments, the ASO comprises or consists of one of SEQ ID NOs: 83, 84, 85 and 87, optionally having one, two or three substitutions.

In certain embodiments, the ASO comprises or consists of one of SEQ ID NOs: 44, 46, 47, 48, 83, 84, 85, 86 and 87 optionally having one, two or three substitutions.

The ASO described herein may comprise an LNA, RNA or DNA nucleotide. In some embodiments, the ASO comprises alternating LNA and RNA nucleotides. In other embodiments, the ASO comprises alternating LNA and DNA nucleotides. In specific embodiments, the ASO comprises alternating RNA and DNA nucleotides.

In some embodiments, the ASO comprises a backbone modification, which is optionally a phosphorothioate linkage. In some embodiments, the ASO comprises a sugar moiety modification, which is optionally 2′-O-methoxyethyl (MOE) modification. Preferably, the ASO comprises a 2′-O-methoxyethyl (MOE) modification and a phosphorothioate linkage. In specific embodiments, the ASO is fully 2′-O-methoxyethyl and fully phosphorothioate modified. In some embodiments, the ASO comprises a 2′-O-methyl modification. In some embodiments, the ASO is fully 2′-O-methyl and fully phosphorothioate modified.

In certain embodiments, the ASO is or comprises a phosphorodiamidate morpholino oligonucleotide.

In another aspect, the present invention provides a method for inhibiting non-sense mediated decay of RBM3-encoding mature mRNA in a cell, the method comprising exposing the cell to an agent, wherein the agent is an ASO described herein.

In another aspect, provided is a method for treating or preventing a disease affected by RMB3 expression in a subject, the method comprising administering to the subject an agent, wherein the agent is an ASO described herein. Also provided is a method for increasing neuroprotection in a subject, the method comprising administering to the subject an agent, wherein the agent is an ASO described herein.

The invention further provides an agent for use in a method for inhibiting nonsense-mediated decay (NMD) of RBM3-encoding mature mRNA, for use in a method for treating or preventing a disease affected by RMB3 expression, or for use in a method for increasing neuroprotection in a subject as described herein. In some embodiments, the agent is an ASO.

In another aspect, provided is use of an agent in the preparation of a medicament for use in a method for inhibiting nonsense-mediated decay (NMD) of RBM3-encoding mature mRNA, use of an agent in the preparation of a medicament for use in a method for treating or preventing a disease affected by RMB3 expression, or use of an agent in the preparation of a medicament for increasing neuroprotection in a subject, as described herein.

In a further aspect, provided is a method of identifying an antisense oligonucleotide (ASO) capable of increasing expression of RBM3 in a cell, the method comprising i) identifying an ASO that targets a region of the pre-mRNA of the RBM3 gene, wherein the region is selected from: a region within exon 3a, a region spanning a splice site of exon 3a, a region located within 250 nucleotides upstream exon 3a and a region located within 250 nucleotides downstream exon 3a; ii) delivering the ASO identified in step i) to the cell; and iii) measuring the level of expression of RBM3 in the cell of step ii). In certain embodiments, the method further comprises: iv) comparing the level of expression measured in step iii) with the level of expression of RBM3 in a cell treated with a control, which is optionally an ASO or DMSO. Preferably, the level of expression is measured by RT-qPCR or by western blotting.

In another aspect, the present invention provides the CRISPR/Cas-based system as described herein including at least one gRNA capable of hybridising to a region in the gene of RBM3 such as to remove exon 3a therefrom.

In another aspect, the present invention provides the CRISPR/Cas-based base editing system as described herein including at least one gRNA capable of hybridising to a region in the gene of RBM3 such as to edit one or more of the splice sites described herein,

In another aspect, provided is a pair of guide RNAs for removing exon 3a from the gene of RBM3, wherein the first guide RNA is capable of hybridising to the genomic sequence upstream of exon 3a and the second guide RNA is capable of hybridising to the genomic sequence downstream of exon 3a. In some embodiments, the first guide RNA comprises SEQ ID NO: 11 or SEQ ID NO: 12 and the second guide RNA comprises SEQ ID NO: 13.

In some embodiments, the gRNA is complementary or essentially complementary to all or part of the region defined above. In certain embodiments, the gRNA is 80%, 85%, 90%, 95% or 100% complementary to said region. In certain embodiments, the gRNA is 100% complementary to said region.

Also provided is an expression construct, or combination of constructs, encoding the CRISPR/Cas system. Preferably the expression construct is a vector or a viral vector.

In another aspect, the present invention provides a method of treating or preventing a disease affected by RMB3 expression in a subject, or providing neuroprotective treatment to a subject, the method comprising administering to the subject the CRISPR/Cas-based base editing system described herein, or the expression construct(s) encoding the system.

The invention includes the combination of the aspects and preferred features described except where such a combination is clearly impermissible or expressly avoided.

### DETAILED DESCRIPTION OF THE INVENTION

### RBM3

The RNA-binding motif protein 3 (RBM3), also known as IS1-RNPL and RNPL, is a cold shock protein which is up-regulated in response to decreased temperature.¹ RBM3 is an evolutionary highly conserved RNA binding protein.

The human RBM3 gene (NCBI gene ID: 5935) is located at 48574484-48581162 chromosome X, GRCh38.p13 Primary Assembly and encodes a 157 amino acid long RBM3 protein (NCBI Reference Sequence: NP_006734.1). The mouse RBM3 gene has NCBI Gene ID: 19652.

RBM3 is expressed in tissues such as bone marrow and brain tissues (https://www.ncbi.nlm.nih.gov/gene?Db=gene&Cmd=ShowDetailView&TermToSearch=5935#g ene-expression). Multiple alternatively spliced transcript variants that are predicted to encode different isoforms have been characterised.

In this specification “RBM3” refers to a RBM3 from any species and includes isoforms, fragments, variants or homologues of RBM3 from any species. In certain embodiments the species is human (Homo sapiens). In some embodiments the species is mouse (Mus musculus).

“Variants or homologues” of RBM3 or its encoding nucleic acid refers to the native RBM3 in the relevant species which corresponds functionally to the human sequence, and shares a high degree of sequence similarity or identity such that the two can be optimally aligned and corresponding regions identified as described herein.

### Alternative Splicing of Pre-mRNA

Most unspliced eukaryotic pre-mRNA consists of protein-coding exons and intervening noncoding introns. During pre-mRNA splicing, introns are excised from the pre-mRNA and exons are connected to form the mature mRNA, which is then translated into a protein. The boundaries between introns and exons are marked by splice sites (ss) at the intron-exon junction.

Over 95% of human multiexon genes can be spliced in a way that multiple different mature mRNAs are created (Pan, Q. et al. (2008), Deep surveying of alternative splicing complexity in the human transcriptome by high-throughput sequencing., Nature genetics, 40(12), pp. 1413-5. doi: 10.1038/ng.259; Barbosa-Morais, N. L. et al. (2012) The evolutionary landscape of alternative splicing in vertebrate species., Science 338(6114), pp. 1587-93. doi:10.1126/science. 1230612; Merkin, J. et al. (2012) Evolutionary dynamics of gene and isoform regulation in Mammalian tissues., Science. 338(6114), pp. 1593-9. doi:10.1126/science. 1228186.). In contrast to constitutive splicing, where only one mRNA isoform is created from a gene, this process is called alternative splicing (AS). AS can be classified into five major categories: shortening of an exon by using an alternative 5′ or 3′ splice site (A5ss/A3ss), skipping of a cassette exon (SE), skipping of either one of two consecutive exons and simultaneous inclusion of the other (mutually exclusive exons, MXE) or retaining of an intron (RI).

Alternative splicing factors regulate AS by interacting with components of the spliceosome and can either promote or repress the usage of a particular splice site. These trans-acting protein factors bind the pre-mRNA at cis-regulatory elements within its sequence. Cis-regulatory elements can be divided into four categories based on their position and type of regulation: intronic splicing enhancers (ISE), exonic splicing enhancers (ESE, both promote ss usage), intronic splicing silencers (ISS) and exonic splicing silencers (ESS, both repress ss usage) (Black, D. L. (2003) Mechanisms of Alternative Pre-Messenger RNA Splicing, Annual Review of Biochemistry. pp. 291-336. doi: 10.1146/annurev.biochem.72.121801.161720.).

One example of alternative splicing events that control mRNA levels and therefore protein levels are highly conserved exons in RNA-binding proteins.⁵ These exons, when included into the transcript, lead to degradation of the mRNA via the nonsense mediated decay (NMD) pathway. Therefore, by preventing inclusion of these exons, one can inhibit nonsense mediated decay (NMD) of the mRNA and increase the mRNA and protein levels of an RNA-binding protein.

In all embodiments herein, referring to ‘mRNA’, ‘RNA’ or ‘transcript’ means a messenger RNA molecule which may be the primary transcript (pre-mRNA), or the product of processing and splicing of pre-mRNA, termed mature mRNA. Pre-mRNAs have not been spliced and therefore include introns.

### Nonsense-Mediated Decay (NMD)

Nonsense-mediated mRNA decay (NMD) is a eukaryotic RNA degradation pathway through which a set of NMD factors recognize and degrade mRNA isoforms that contain translation termination codons that are positioned in abnormal contexts. These codons are called premature termination codons (PTCs). A PTC-containing mRNA isoform is often called a poison isoform.

Poison isoforms are frequently found in RNA binding proteins where alternative splicing leads to inclusion of a PTC-containing exon, a poison exon, into the transcript.⁵ A heat-induced poison exon of CIRBP provides an explanation for cold-induced CIRBP expression.⁵

PTC recognition occurs during translation of the mRNA and requires several proteins to ultimately degrade the mRNA. These proteins include NMD factors such as UPF1, UPF2 and UPF3 and SMG6 and SMG5-SMG7.⁶ The process of NMD is described in greater detail in Lykke-Andersen, S. & Jensen, T. H. Nature Reviews Molecular Cell Biology 16, 665 (2015), hereby incorporated by reference in its entirety.

An inhibitor of translation may be used to stabilise the poison mRNA isoforms before degradation occurs thus allowing the accumulation and detection of poison mRNA isoforms. An exemplary inhibitor of translation is cycloheximide (CHX). Alternatively, NMD may be inhibited by knocking down UPF1 and/or other proteins that play a role in NMD, such as SMG6 and SMG7.

### RBM3 Exon 3a

The mature mRNA of human RBM3 that encodes the RBM3 polypeptide (NP_006734.1) is defined in GenBank accession no. NM_006743.5 and consists of seven annotated exons. Exon 3 (SEQ ID NO: 1) extends from nucleotide 207 to 313, and exon 4 (SEQ ID NO: 2) extends from nucleotide 314 to 419 of NM_006743.5.

The mature mRNA of mouse RBM3 that encodes RBM3 polypeptide isoform 1 (NP_001159881.1) is defined in GenBank accession no. NM_001166409.2 and consists of seven annotated exons. Exon 3 (SEQ ID NO: 3) extends from nucleotide 362 to 468 and exon 4 (SEQ ID NO: 4) extends from 469 to 568 of NM_001166409.2.

The inventors identified an alternative splicing event that leads to inclusion of a previously unannotated exon, referred to herein as exon 3a, into the RBM3 transcript. Exon 3a contains PTCs and induces nonsense-mediated decay (NMD) of the transcript thus leading to decreased mRNA levels and protein levels of RBM3. The inventors further identified that inclusion of exon 3a is induced at warm temperatures.

Exon 3a is located within the annotated intron 3, between annotated exons 3 and 4 of RBM3. Table 1 demonstrates the sequence of exon 3a as mapped onto the mouse and human genomes. The two sequences share high (91%) sequence identity.

The present invention provides methods of modulating splicing and thereby avoiding incorporation of Exon 3a (or at least its associated PTCs) into mature RBM3 mRNA. Avoiding incorporation of exon 3a means that all or part of exon 3a is not incorporated into mature RBM3 mRNA. As described in Example 3, there is an alternative, internal 3′ss at position 107 of the sequence of exon 3a (e.g. SEQ ID NO: 6 and SEQ ID NO: 5) and therefore the agents and methods of the invention may lead to mature mRNA wherein nucleotides 108 to 271 of SEQ ID NO: 5 or nucleotides 108 to 269 of SEQ ID NO:6 are not incorporated (they are skipped). Therefore the agents of the present invention may result in skipping of all of exon 3a or part of exon 3a, for example nucleotides 108 to 269 of SEQ ID NO:6.

TABLE 1
Sequence and position of exon 3a
in human and mouse genomes

| | Position of | |
|---|---|---|
| | exon 3a in | |
| Genome | Genome | Sequence of exon 3a |
| Mouse | chrX: | GGGGTGGAGGGCAGCGGCAGACAGAGCCG |
| (GRCm39) | 8010684- | GGCTGCCTGGGAGGCGACGACTGGTCCTG |
| | 8010414 | CATGAGGCGGAGAAGCCACCTGTGGATGG |
| | | TTGACCTGCTCTGGGCTTAGGTAGTAGAG |
| | | TCTGCAGAGCTGTGGGCCTGGCAGAGGAC |
| | | CGGAGAGGACCTCGTGAGTCTGCTGGGAT |
| | | GGACTCTGAAGCCTCAGACCGAGAGAGGT |
| | | GTCTAGCCCTAATAGTCGGGCAGCTTAGA |
| | | GGAGTGGGGAGGATACGTCAGCCTGAGTT |
| | | CAGTCAAGAT |
| | | (SEQ ID NO: 5) |
| Human | X | GGGGTGGAGGGCAGCGGCAGACAGAGCCT |
| (GRCh38) | chromosome, | GGCCGCCTGGGAGGCGACGACTGGTCCTG |
| | 48575815- | CATGAGGCCGAGAAGCCACCTGTGGATGG |
| | 48576083 | TTGACCTGCCTTGGGCTTAGGTAGTAGAG |
| | | TCTGCAGACCTGTGGGCCTGGCCCGGAGA |
| | | GGACCTCGTGAGTCTTCTGACATGGACTC |
| | | TGAAGCCTCAAACCAAGAGAGGTGTCTGT |
| | | CTAGCCCTGATAGTCGGGCAGCTTAGAGG |
| | | AGTGGGGAGGAGAACTCAGCCTGAGTTCA |
| | | GTCAAGAT |
| | | (SEQ ID NO: 6) |

In certain embodiments the species is human (Homo sapiens) and therefore “exon 3a” refers to SEQ ID NO: 6.

In some embodiments the species is mouse (Mus musculus) and therefore “exon 3a” refers to SEQ ID NO: 5.

RBM3 is highly conserved across species (Zhou R B et al. Oncotarget. 2017; 8(13): 22235-22250). In view of the high evolutionary conservation of RBM3, exon 3a may be mapped onto the genomes of other species using known alignment techniques.

Thus “exon 3a” as used herein may comprise the sequence of exon 3a of the human RBM3 gene (SEQ ID NO: 6) or the sequence of exon 3a of the mouse RBM3 gene (SEQ ID NO: 5), or may comprise a sequence which is a homologue or other variant of SEQ ID NO: 6 or SEQ ID NO: 5. In the context of the claimed invention the RBM3 exon 3a will typically be that native to the relevant cell or subject.

### Detection of Exon 3a

The presence of exon 3a in the mRNA of RBM3 may be detected by using PCR primers that are complementary to sequences upstream and downstream of exon 3a, e.g. binding to exons 3 and 4 or 5 or intronic sequences upstream and downstream of exon 3a. As discussed above, inhibition of translation may be employed to stabilise the exon 3a-containing mRNA isoform before degradation via NMD occurs.

Thus in another aspect, a pair of PCR primers is provided. In some embodiments, the PCR primers are forward primer (5′-TCATCACCTTCACCAACCCA (SEQ ID NO: 7)) and a reverse primer (5′-TCTAGAGTAGCTGCGACCAC (SEQ ID NO: 8)). In some embodiments, the PCR primers are forward primer (5′-TCATCACCTTCACAAACCCA (SEQ ID NO: 9)) and a reverse primer (5′-GTGGTCGCAGTTACTCTAGA (SEQ ID NO: 10)), which are designed for mouse RBM3 gene.

### Genome Editing

Genome editing may be used to generate cell lines lacking RBM3 exon 3a or as a therapy for treating or preventing a disease as described herein. Various genome editing techniques may be used, for example CRISPR/Cas9, to remove exon 3a from the gene of RBM3. A pair of guide RNAs may be used to remove exon 3a. An engineered cell lacking exon 3a may exhibit higher expression of RBM3 even at low temperatures, for example as shown in the specific embodiment of Example 5.

In another aspect, an engineered cell lacking exon 3a is provided. In certain embodiments, expression of RBM3 in the engineered cell is higher as compared to expression of RBM3 in a wildtype (or other control) cell. In some embodiments, expression of RBM3 at 32° C., 33° C., 34° C., 35° C., 36° C., 37° C., 38° C., 39° C., 40° C., or 41° C. is higher in the engineered cell as compared to expression of RBM3 in a wildtype (or other control) cell.

In one aspect, a pair of guide RNAs for removing exon 3a from the gene of RBM3 are provided, optionally wherein the first guide RNA is capable of hybridising to the gene upstream of exon 3a and the second guide RNA is capable of hybridising to the gene downstream of exon 3a. In some embodiments, the first guide RNA comprises SEQ ID NO: 11 or SEQ ID NO: 12 and the second guide RNA comprises SEQ ID NO: 13.

### Base Editing

CRISPR-mediated base editing may be used to alter the splice sites of RBM3 exon 3a, which will result in permanent exon exclusion (non-incorporation).

The Cas protein forms a complex with the 3′ end of a gRNA. The specificity of the CRISPR-based system depends on two factors: the targeting sequence and the protospacer-adjacent motif (PAM). The targeting or recognition sequence is located on the 5′ end of the gRNA and is designed to pair with base pairs on the host DNA (target nucleic acid or target DNA) at the correct DNA sequence known as the protospacer. By simply exchanging the recognition sequence of the gRNA, the Cas protein can be directed to new genomic targets. The PAM sequence is located on the DNA to be altered and is recognized by a Cas protein. PAM recognition sequences of the Cas protein can be species specific.

As is well known in the art (see e.g. Kluesner, Mitchell G., et al. “CRISPR-Cas9 cytidine and adenosine base editing of splice-sites mediates highly-efficient disruption of proteins in primary and immortalized cells.” Nature communications 12.1 (2021): 1-12) base editors are a class of gene editing enzymes that consist of a Cas protein fused to a base-editing domain, for example Cas9 nickase fused to a nucleotide deaminase domain. In principle, base editors localize to a target region in the genome guided by a gRNA. Once bound, the Cas9 complexes displaces the non-bound strand, forming a ssDNA R-loop. The R-loop is rendered accessible to the tethered deaminase domain, whereby cytidine deaminase base editors (CBEs, C:G-to-T:A) deaminate C-to-U, which base pairs like T, and adenosine deaminase base editors (ABEs, A:T-to-G:C) deaminate A-to-I, which base pairs like G. Concurrent nicking of the unedited strand by the core Cas9 nickase then stimulates DNA repair to use the newly deaminated base as a template for DNA polymerization, thereby preserving the edit in both strands of the DNA.

Examples of therapeutic base editing are disclosed, for example, in WO2022081612A1.

In some embodiments, the base-editing domain includes an adenosine deaminase base editor (ABE). Adenine base editors may include, for example, ecTadA, including wild-type and mutants thereof. The adenosine deaminase base editor may be as described in Gaudelli et al. (Nature 2017, 551, 464-471), Koblan et al. (Nature Biotech. 2018, 36, 843-846), Richter et al. (Nature Biotech. 2020, 38, 883-891), and Gaudelli et al. (Nature Biotech. 2020, 38, 892-900), each of which is incorporated herein by reference.

In some embodiments, the base-editing domain includes a cytidine deaminase domain. Cytidine deaminase domain can convert the DNA base cytosine to uracil. In some embodiments, the cytidine deaminase domain can include an apolipoprotein B mRNA-editing enzyme, catalytic polypeptide-like (APOBEC) family deaminase. In some embodiments, the cytidine deaminase domain can include an APOBEC 1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, or a combination thereof. In some embodiments, the cytidine deaminase domain comprises an APOBEC 1 deaminase. In some embodiments, the cytidine deaminase domain comprises a rat APOBEC 1 deaminase. In some embodiments, a cytidine deaminase enzyme (for example, rAPOBEC1) can be fused to the N-terminus of dCas to generate a base editing enzyme named BE1.

In some embodiments, the CRISPR/Cas-based base editing system comprises a Cas9 protein, such as a catalytically dead dCas9. Cas9 protein is an endonuclease that cleaves nucleic acid and is encoded by the CRISPR loci and is involved in the Type II CRISPR system. A Cas9 molecule can interact with one or more gRNA molecule and, in concert with the gRNA molecule(s), localizes to a site which comprises a target domain, and in certain embodiments, a PAM sequence. The ability of a Cas9 molecule to recognize a PAM sequence can be determined, for example, using a transformation assay. In some embodiments, the Cas9 protein is from Streptococcus pyogenes. In some embodiments, the Cas9 protein is from Staphylococcus aureus.

In some embodiments, the Cas9 protein may be mutated so that the nuclease activity is reduced or inactivated.

Wild type Cas9 has two active sites (RuvC and HNH nuclease domains) for cleaving DNA, one for each strand of the double helix. However, nickase variants of Cas9 are readily available (e.g., Addgene, plasmid #: 48873) that are only capable of cleaving one strand of the DNA due to catalytic inactivation of the RuvC or HNH nuclease domains. Accordingly, in specific embodiments, the Cas protein comprises a Cas9 nickase. In a preferred embodiment, the Cas protein comprises S. aureus Cas9 D10A nickase. In another embodiment, the Cas protein comprises S. aureus Cas9 H840A nickase.

An inactivated Cas9 protein (“iCas9”, also referred to as “dCas9”) with no endonuclease activity may be targeted to genes in bacteria, yeast, and human cells by gRNAs to silence gene expression through steric hindrance. Exemplary mutations with reference to the S. pyogenes Cas9 sequence to reduce or inactivate nuclease activity include: D10A, E762A, H840A, N854A, N863A and/or D986A. Exemplary mutations with reference to the S. aureus Cas9 sequence to inactivate nuclease activity include D10A and N580A.

The Cas9 protein or mutant Cas9 protein may be from any bacterial or archaea species, such as Streptococcus pyogenes, Staphylococcus aureus, Streptococcus thermophiles, or Neisseria meningitides. In some embodiments, the Cas protein or mutant Cas9 protein is a Cas9 protein derived from a bacterial genus of Streptococcus, Staphylococcus, Brevibacillus, Corynebacter, Sutterella, Legionella, Francisella, Treponema, Filifactor, Eubacterium, Lactobacillus, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter. In some embodiments, the Cas9 protein or mutant Cas9 protein is selected from the group, including Streptococcus pyogenes, Francisella novicida, Staphylococcus aureus, Neisseria meningitides, Streptococcus thermophiles, Treponema denticola, Brevibacillus laterosporus, Campylobacter jejuni, Corynebacterium diphtheria, Eubacterium ventriosum, Streptococcus pasteurianus, Lactobacillus farciminis, Sphaerochaeta globus, Azospirillum, Gluconacetobacter diazotrophicus, Neisseria cinerea, Roseburia intestinalis, Parvibaculum lavamentivorans, Nitratifractor salsuginis, and Campylobacter lari.

### Vectors and Nucleic Acids

In some embodiments, the CRISPR/Cas fusion protein may be provided from nucleic acid (e.g. vector, construct) encoding the fusion protein and optionally the gRNA.

Vectors encoding CRISPR/Cas-based base editing systems are commercially available e.g. from Addgene (see e.g. pCMV-ABE7.10, https://www.addgene.org/102919/). Examples of vectors which include cloning sites for gRNA expression include modified ABE7.10 versions that contain a gRNA expression cassette (see e.g. called ABE7.10_4.1 and ABE7.10_3.1 described in Escobar, Helena, et al. “Base editing repairs an SGCA mutation in human primary muscle stem cells.” JCI insight 6.10 (2021).

### Region to be Edited

In one aspect, a CRISPR/Cas-based base editing system for altering an RNA splice site encoded in the genomic DNA of a subject is provided. The CRISPR/Cas-based base editing system comprises a fusion protein and at least one gRNA, wherein the fusion protein comprises a Cas protein and a base-editing domain, wherein at least one gRNA is capable of hybridising to a region (i.e. a target region) in the gene of RBM3.

In some embodiments, the region is a region spanning the 3′ slice site of exon 3a of RBM3.

In other embodiments, the region is a region spanning the 5′ splice site of exon 3a of RBM3.

In yet other embodiments, the region is a region spanning the internal alternative splice site of exon 3a of RBM3 which is at nucleotide 107 of exon 3a.

In some embodiments, the CRISPR/Cas-based base editing system comprises one gRNA targeting a region spanning the 3′ splice site, a gRNA targeting a region spanning the 5′ splice site, and/or a gRNA targeting the internal alternative splice site of exon 3a.

Splice sites that can be base-edited using the system described herein are shown in bold in the following sequence:

GTGGGGCCTCCTATCTGCAGAGGGACCTTGTCCCCATCTGCGGCTTCCTTCATTCTTTTTG
TTTTTCCCTCTGTGTCTGCTCGGGGCAGCGTGGCCACCAAGCCCCGCAGTGCCCACTCAC

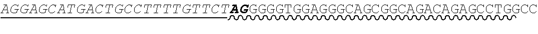

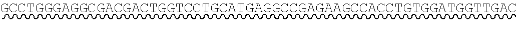

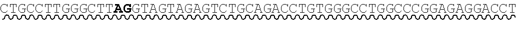

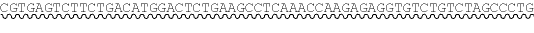

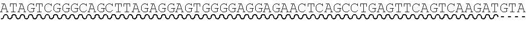

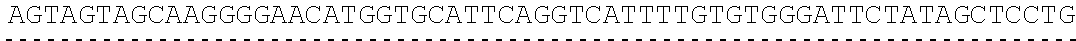

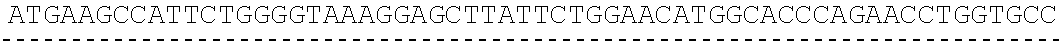

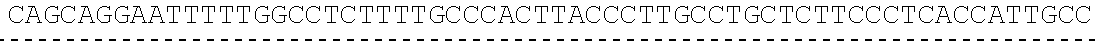

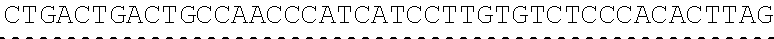

Upstream Intron 3 (SEQ ID NO:32)
Exon 3a (SEQ ID NO: 6)

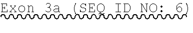

Downstream Intron 3 (SEQ ID NO: 33)
AG is the splice acceptor immediately before the 3′ splice site of exon 3a
AG is internal splice site of exon 3a (nucleotides 106-107)

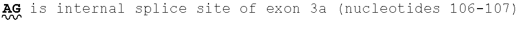

gRNAs

As explained above The CRISPR/Cas-based base editing system includes at least one gRNA. The gRNA targets the RBM3 gene. The gRNA may bind and target a region of the RBM3 gene as described herein. The gRNA may target an RNA splice site in the RBM3 gene. The gRNA provides the targeting of the CRISPR/Cas-based base editing systems. The gRNA may be a fusion of two noncoding RNAs: a crRNA and a tracrRNA. The gRNA may target any desired DNA sequence through complementary base pairing with the desired DNA target, typically across a region of 20 nucleotides. The tracrRNA serves as a binding scaffold for the Cas nuclease.

It will be understood by those skilled in the art that even having selected a splice site for editing, a number of different particular target regions, and particular gRNAs, may be used for effecting the change. This will depend on the PAM site (e.g. “NGG”) utilised by the CRISPR/Cas-based base editing system selected, and the distance of the “editing window” from that site, as well as the edit it is decided to make. In the light of the disclosure herein, and common generally knowledge, those skilled in the art will be able to select suitable gRNAs and systems.

The “target region” refers to the region of the target gene to which the CRISPR/Cas-based gene editing system targets and binds. The portion of the gRNA that targets (hybridises to) the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to the sequence that the gRNA targets. Together, the gRNA targeting portion and gRNA scaffold form one polynucleotide.

The gRNA may comprise at its 5′ end the targeting domain that is sufficiently complementary to the target region to be able to hybridise to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides.

Typically the crRNA, or targeting domain or portion of the gRNA, which hybridises to the target DNA region is 16, 17, 18, 19, 20, or 21 nucleotides long, more preferably 17-20 nucleotides long, optionally 20 nucleotides long.

### Examples of CRISPR/Cas-Based Base Editing Systems and Associated Materials and Methods

In one aspect there is provided a CRISPR/Cas-based base editing system for altering an RNA splice site encoded in the genomic DNA of a subject,
the CRISPR/Cas-based base editing system comprising a fusion protein and at least one guide RNA (gRNA),
wherein the fusion protein comprises a Cas protein and a base-editing domain,
wherein the at least one gRNA is capable of hybridising to a region in the gene of RBM3, and
wherein the region comprises:
(i) nucleotides 136 to 147, 137 to 147, 138 to 147, 139 to 147, 140 to 147, 141 to 147, 142 to 147, 143 to 147, 144 to 147, 145 to 147, or 146 to 147 of SEQ ID NO: 32, or a complement thereof, such that the splice site located at nucleotides 146-147 of SEQ ID NO: 32 is altered;
(ii) SEQ ID NO: 6, or a complement thereof, such that the splice site located at nucleotides 106-107 of SEQ ID NO: 6 is altered; or
(iii) nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33, or a complement thereof, such that the splice site located at the first two nucleotides of SEQ ID NO: 33 is altered,
optionally wherein the gRNA, or the targeting domain of the gRNA which hybridises to the target region, is 16, 17, 18, 19, 20, or 21 nucleotides long.

In one embodiment the region comprises nucleotides 136 to 147, 137 to 147, 138 to 147, 139 to 147, 140 to 147, 141 to 147, 142 to 147, 143 to 147, 144 to 147, or 145 to 147 of SEQ ID NO: 32, or a complement thereof such that the splice site located at the last two nucleotides of SEQ ID NO: 32 is altered, and the region further comprises nucleotides 1 and 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of SEQ ID NO: 6, or a complement thereof.

In one embodiment the region comprises nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33, or a complement thereof, such that the splice site located at the first two nucleotides of SEQ ID NO: 33 is altered, and the region further comprises nucleotides 254 to 269, 255 to 269, 256 to 269, 257 to 269, 258 to 269, 259 to 269, 260 to 269, 261 to 269, 262 to 269, 263 to 269, 264 to 269, 265 to 269, 266 to 269, or 267 to 269 of SEQ ID NO: 6.

In one embodiment the region spans nucleotides 106-17 of SEQ ID NO: 6 and comprises 5, 6, 7, 8, 9, 10, 11, 12, 13, 14, 15, 16, 17, 18, 19, or 20 contiguous nucleotides, such that the splice site located at nucleotides 106-107 of SEQ ID NO: 6 is altered.

Thus in some embodiments, the presently disclosed CRISPR/Cas-based base editing system is for altering a splice site by converting “AG” (splice acceptor) to an “AA” or “GG” which promotes skipping of exon 3a. In other embodiments, the CRISPR/Cas-based base editing system allows for altering of a splice site by converting “GT” (splice donor) to an “GC” or “AT” which promotes skipping of exon 3a.

Adenosine or Cytidine base editing on the coding or noncoding DNA strand may be used to alter the 3′ splice site of Rbm3 exon 3a (last two nucleotides of SEQ ID NO: 32 in human or SEQ ID NO: 30 in mouse), the 5′ splice site of Rbm3 exon 3a (first two nucleotides of SEQ ID NO: 33 in human or SEQ ID NO: 31 in mouse) and/or the internal 3′ splice site at position 106-107 in SEQ ID NO: 6 (human) or SEQ ID NO: 5 (mouse).

In some embodiments the base-editing domain includes an adenosine deaminase base editor which converts the “AG” of positions 146 to 147 of SEQ ID NO: 32 to “GG”.

In some embodiments, the gRNA hybridises to a region spanning the 3′ splice site of exon 3a. In specific embodiments the gRNA comprises sequence TTCTaGGGGGTGGAGGGCAG (SEQ ID NO: 90) or a complement thereof, optionally wherein the sequence has one, two, or three nucleotide substitutions.

In some embodiments SEQ ID NO: 90 is used with an adenosine deaminase base editor which converts the “AG” of positions 146 to 147 of SEQ ID NO: 32 to “GG”.

In some embodiments the base-editing domain includes an adenosine deaminase base editor which converts the “AG” of the internal splice site of exon 3a (nucleotides 106-107).

In some embodiments, the gRNA hybridises to a region spanning this splice site of exon 3a. In specific embodiments the gRNA comprises sequence CTACTACCTAAGCCCAAGGC (SEQ ID NO: 91) or a complement thereof, optionally wherein the sequence has one, two, or three nucleotide substitutions. Such gRNA sequences can bind to the reverse DNA strand and target the ‘C’ that is complementary to the AG.

In some embodiments the base-editing domain includes an adenosine deaminase base editor which converts the “ [symbol] ” splice donor immediately following the 5′ splice site of exon 3a.

In some embodiments, the gRNA hybridises to a region spanning this splice site. In specific embodiments the gRNA comprises sequence CTTACATCTTGACTGAACTC (SEQ ID NO: 92) or a complement thereof, optionally wherein the sequence has one, two, or three nucleotide substitutions. Such gRNA sequences can bind to the reverse DNA strand and target the ‘C’ or ‘A’ complementary to GT with an ABE or a CBE.

Therefore in one embodiment the target region is selected from SEQ ID NOs: 90, 91, and 92, and the at least one gRNA comprises or consists of a sequence which is the RNA equivalent of SEQ ID NOs: 90, 91, and 92 or a complement thereof.

In one embodiment altering the RNA splice site encoded in the genomic DNA results in non-incorporation of exon 3a in the resulting mature mRNA.

In one embodiment, the Cas protein comprises Cas9, optionally wherein the Cas protein comprises a Cas9 nickase.

In one embodiment, the base-editing domain comprises a cytidine deaminase domain or an adenosine deaminase domain.

In one embodiment, there is provided an isolated nucleic acid encoding and capable of expressing a guide RNA capable of hybridising to a region in the gene of RBM3, wherein the isolated nucleic acid comprises a sequence selected from SEQ ID NOs: 90, 91 or 92, or a complement thereof.

Also provided are one or more isolated polynucleotides encoding the CRISPR/Cas-based base editing system described herein, including the gRNA.

In one embodiment the polynucleotide comprises a first polynucleotide encoding the fusion protein and a second polynucleotide encoding the at least one gRNA.

Also provided is an expression construct, which is optionally a vector, which is optionally a viral vector, comprising the isolated polynucleotide encoding the CRISPR/Cas system described above.

Also provided is a cell comprising the isolated polynucleotide or the expression construct.

Also provided is a composition comprising the CRISPR/Cas-based base editing system, isolated polynucleotide(s), or the expression construct.

### Methods of Using CRISPR/Cas-Based Base Editing Systems

In another aspect, provided is a method for inhibiting non-sense mediated decay of RBM3-encoding mature mRNA in a cell and/or for increasing expression of RBM3 in a cell, the method comprising exposing the cell to the CRISPR/Cas-based base editing system described herein, or a polynucleotide encoding the CRISPR/Cas-based base editing system, such as to alter a splice site of exon 3a.

In another aspect there is provided a method of treating or preventing a disease affected by RMB3 expression in a subject, or providing neuroprotective treatment to a subject, the method comprising administering to the subject the CRISPR/Cas-based base editing system, the isolated nucleic acid, the expression construct, or the composition described above.

In one embodiment the treating or treatment is for:
a neurological disease and/or the disease is neonatal hypoxic ischemic encephalopathy, head injury, or stroke;
a neurodegenerative disease optionally selected from Alzheimer's disease, Parkinson's disease, prion disease, frontotemporal dementia, a tauopathy, amyotrophic lateral sclerosis (ALS), and vascular dementia; or
neurological damage, optionally caused during cardiac surgery or induced coma; or
depression or anxiety.

### Agents Capable of Inducing Expression of RBM3

In accordance with various aspects of the present invention, in some embodiments the agent is an agent capable of inhibiting alternative splicing (inhibiting inclusion of exon 3a) of RBM3 pre-mRNA. In certain embodiments, the agent is an agent capable of inducing skipping of exon 3a. The agent may be exogenous to the cell.

The agent is typically capable of binding to a specific region of the pre-mRNA transcript and modulating or altering splicing of the pre-mRNA.

In accordance with various aspects of the present invention, in some embodiments the agent is capable of hybridising to a region of the pre-mRNA of RBM3 to alter the splicing of the pre-mRNA such that a premature termination codon (PTC) is not included between exons 3 and 4 in the resulting mature mRNA. In some embodiments the agent is complementary to the pre-mRNA of RBM3 to alter the splicing of the pre-mRNA such that a premature termination codon (PTC) is not included between exons 3 and 4 in the resulting mature mRNA. In certain embodiments, splicing is altered such that exon 3a is not incorporated (it is skipped) in the resulting mature mRNA.

In accordance with various aspects of the invention, in some embodiments, the agent is useful in altering an RNA splice site encoded in the genomic sequence of RBM3. Accordingly, where appropriate, an “agent” may refer to a CRISPR/Cas-based base editing system as described herein, a guide RNA as described herein, or a polynucleotide/expression construct encoding the CRISPR/Cas-based base editing system of the invention. Where it is stated that the target splice site is “altered” by the systems disclosed herein, it will be understood that such systems are adapted for, and capable of, altering the splice sites, when introduced into cells comprising the target genomic DNA under appropriate conditions.

In some embodiments, the agent is capable of hybridising to a target nucleic acid (e.g., pre-mRNA) sequence by Watson-Crick base pairing or wobble base pairing (G-U). The agent may have exact sequence complementarity to the target sequence or near complementarity, for example be essentially complementary (e.g., sufficient complementarity to bind the target sequence and alter splicing of the pre-mRNA). The agent (e.g. gRNA) may have exact sequence complementarity to the target sequence or near complementarity, for example be essentially complementary (e.g., sufficient complementarity to bind the target sequence and enable editing of a splice site).

In this specification ‘resulting mature mRNA’ means the product of splicing of pre-mRNA. It does not necessarily mean all of the mature mRNA, the agent may alter splicing such that at least some of the mature mRNA has the above-mentioned properties.

In some embodiments the agent is an agent capable of hybridising to (or is complementary to) exon 3a or a region spanning a splice site thereof in the pre-mRNA. In certain embodiments, the agent is capable of hybridising to (or is complementary to) a region spanning the 3′ splice site of exon 3a in the pre-mRNA. In some embodiments, the agent is capable of hybridising to (or is complementary to) a region spanning the 5′ splice of exon 3a in the pre-mRNA. In certain embodiments, the agent is capable of hybridising to (or is complementary to) a region located within 250 nucleotides upstream exon 3a. In certain embodiments, the agent is capable of hybridising to (or is complementary to) a region located within 250 nucleotides downstream exon 3a.

In some embodiments, the agent is capable of hybridising to (or is complementary to) a region located within 200 nucleotides upstream exon 3a. In some embodiments, the agent is capable of hybridising to (or is complementary to) a region located within 200 nucleotides downstream exon 3a. The region may be within 10, 20, 30, 40, 50, 60, 70, 80, 90 or 100 nucleotides upstream exon 3a. The region may be within 10, 20, 30, 40, 50, 60, 70, 80, 90 or 100 nucleotides downstream exon 3a. The region may be within 110, 120, 130, 140, 150, 160, 170, 180, 190 or 200 nucleotides upstream exon 3a. The region may be within 110, 120, 130, 140, 150, 160, 170, 180, 190 or 200 nucleotides downstream exon 3a. The region may be within 210, 220, 230, 240 or 250 nucleotides upstream exon 3a. The region may be within 210, 220, 230, 240 or 250 nucleotides downstream exon 3a. The region may be upstream or downstream exon 3a and corresponding to annotated intron 3 of the RBM3 gene, for example a region corresponding to the sequences shown in Table 3 hereinbelow.

In some embodiments the agent is a nucleic acid and/or nucleic acid analogue, such as an oligonucleotide or a polynucleotide. In some embodiments, the agent is an antisense oligonucleotide (ASO).

One embodiment of the present disclosure is a composition comprising nucleic acids and/or nucleic acid analogues, such as a composition comprising a polynucleotide, that inhibit nonsense mediated mRNA decay (NMD) of RBM3.

The nucleic acids or polynucleotides are typically antisense oligonucleotides (ASOs) that are capable of hybridising to a specific region of the pre-mRNA transcript and modulate or alter splicing. An ASO may target an alternative splice site and block its use by the spliceosome. In some embodiments, the ASO targets regulatory sequences in close proximity or overlapping the 5′ splice site or 3′ splice site of exon 3a. Exon ligation at the 5′ splice site of the human and mouse exon 3a takes place at the last nucleotide of the sequences shown in Table 1 and FIG. 1E (see also FIG. 4A). Exon ligation at the 3′ splice site is at the first nucleotide of the sequences of exon 3a shown in Table 1 and FIG. 1E.

An agent (e.g. ASO) may interfere with the binding of trans-acting factors to a cis-regulatory element in the pre-mRNA. Therefore, the agent may target a cis-regulatory element that promotes or induces inclusion of exon 3a (splice enhancer element). In certain embodiments, the agent targets (e.g. is capable of hybridising to) a splice enhancer element.

The splice enhancer element may be located within exon 3a as described herein.

The splice enhancer element may be located in a region upstream or downstream of exon 3a, as described herein. Preferably, the splice enhancer element is located within 250 nucleotides upstream or downstream of exon 3a. The splice enhancer element may be located at a region between exon 3 and exon 4 which corresponds to annotated intron 3 of the RBM3 gene. In some embodiments, the splice enhancer element is located within 200 nucleotides upstream exon 3a. In some embodiments, the splice enhancer element is located within 200 nucleotides downstream exon 3a. The splice enhancer element may be within 10, 20, 30, 40, 50, 60, 70, 80, 90 or 100 nucleotides upstream exon 3a. The splice enhancer element may be within 10, 20, 30, 40, 50, 60, 70, 80, 90 or 100 nucleotides downstream exon 3a. The splice enhancer element may be within 110, 120, 130, 140, 150, 160, 170, 180, 190 or 200 nucleotides upstream exon 3a. The splice enhancer element may be within 110, 120, 130, 140, 150, 160, 170, 180, 190 or 200 nucleotides downstream exon 3a. The splice enhancer element may be within 210, 220, 230, 240 or 250 nucleotides upstream exon 3a. The splice enhancer element may be within 210, 220, 230, 240 or 250 nucleotides downstream exon 3a.

Regulatory elements such as splice enhancers and silencers may be identified by screening mutagenesis along regions of RBM3 gene and measuring the inclusion level of exon 3a in the transcript of each mutant with respect to the wildtype gene. An exemplary method is described in Example 3.

In another aspect, a method of identifying a cis-regulatory element capable of controlling inclusion of exon 3a in a transcript of RBM3 is provided. The method comprises
i) mutating or deleting a set of nucleotides in a region within exon 3a, a region within 250 nucleotides upstream or a region within 250 nucleotides downstream of exon 3a in RBM3 gene,
ii) measuring the level of inclusion of exon 3a in the transcript, and
iii) comparing the level of inclusion of step ii) to the level of inclusion in the transcript of a wildtype gene. The level of inclusion of exon 3a may be measured by splicing-sensitive RT-PCR.

In certain embodiments, the agent (e.g. ASO) targets a region comprising a splice enhancer element. In specific embodiments, the region is selected from: M2, M2-2, M2-2, M2-3, M2-4, M2-6, M2-7 and M2-9, M4, and M4-7. In some embodiments, the region is M4-7. In some embodiments, the region is M2-9. In other embodiments, the region corresponds to the “M2 core” region (SEQ ID NO: 88) or to the “M4 core” region (SEQ ID NO: 89). Table 2 provides the sequence, SEQ ID NO and location of each region.

TABLE 2
Regions in RBM3 exon 3a that comprise a splice enhancer element.

| Description | RBM3 gene | Nucleotide Sequence |
|---|---|---|
| M2 | Mouse | CTGCCTGGGAGGCGACGACTGGTCCTGCATGA |
| | (chrX: 8010653- | GGCGGAGAAGCCACCTGT |
| | 8010604) | (SEQ ID NO: 14) |
| | Human | CCGCCTGGGAGGCGACGACTGGTCCTGCATGA |
| | (chrX: 48575846- | GGCCGAGAAGCCACCTGT |
| | 48575895) | (SEQ ID NO: 15) |
| M2-2 | Mouse | GGCGACGACT |
| | Human | (SEQ ID NO: 16) |
| M2-3 | Mouse | GGTCCTGCAT |
| | Human | (SEQ ID NO: 17) |
| M2-4 | Mouse | GAGGCGGAGA |
| | | (SEQ ID NO: 18) |
| | Human | GAGGCCGAGA |
| | | (SEQ ID NO: 19) |
| M2-6 | Mouse | CTGCCTGGGAGGCGACGACT |
| | | (SEQ ID NO: 20) |
| | Human | CCGCCTGGGAGGCGACGACT |
| | | (SEQ ID NO: 21) |
| M2-7 | Mouse | GGTCCTGCATGAGGCGGAGA |
| | | (SEQ ID NO: 22) |
| | Human | GGTCCTGCATGAGGCCGAGA |
| | | (SEQ ID NO: 23) |
| M2-9 | Mouse | GGCGACGACTGGTCCTGCATGAGGCGGAGA |
| | | (SEQ ID NO: 24) |
| | Human | GGCGACGACTGGTCCTGCATGAGGCCGAGA |
| | | (SEQ ID NO: 25) |
| M4 | Mouse | GCCTGGCAGAGGACCGGAGAGGACCTCGTGAG |
| | (chrX: 8010553- | TCTGCTGGGATGGACTCT |
| | 8010504) | (SEQ ID NO: 26) |
| | Human | CTGTGGGCCTGGCCCGGAGAGGACCTCGTGAG |
| | (chrX: 48575940- | TCTTCTGACATGGACTCT |
| | 48575989) | (SEQ ID NO: 27) |
| M4-7 | Mouse | GGACCTCGTGAGTCTGCTGG |
| | | (SEQ ID NO: 28) |
| | Human | GGACCTCGTGAGTCTTCTGA |
| | | (SEQ ID NO: 29) |
| M4-1 | Mouse | Gcctggcaga |
| | | (SEQ ID NO: 72) |
| | Human | CTGTGGGCCT |
| | | (SEQ ID NO: 73) |
| M4-2 | Mouse | Ggaccggaga |
| | | (SEQ ID NO: 74) |
| | Human | GGCCCGGAGA |
| | | (SEQ ID NO: 75) |
| M4-3 | Mouse | Ggacctcgtg |
| | Human | (SEQ ID NO: 76) |
| M4-4 | Mouse | Agtctgctgg |
| | | (SEQ ID NO: 77) |
| | Human | AGTCTTCTGA |
| | | (SEQ ID NO: 78) |
| M4-5 | Mouse | Gatggactct |
| | | (SEQ ID NO: 79) |
| | Human | CATGGACTCT |
| | | (SEQ ID NO: 80) |
| M4-6 | Mouse | Gcctggcagaggaccggaga |
| | | (SEQ ID NO: 81) |
| | Human | CTGTGGGCCTGGCCCGGAGA |
| | | (SEQ ID NO: 82) |

In certain embodiments, the agent (e.g. ASO) targets a region spanning a splice site of exon 3a. In certain embodiments, the region spans the 5′ splice site of exon 3a, for example the region may comprise nucleotides (nt) 259 to 271, 260 to 271, 261 to 271, 262 to 271, 263 to 271, 264 to 271, 265 to 271, 266 to 271, 267 to 271, 268 to 271, or 269 to 271 of SEQ ID NO: 5. Accordingly, the region in the human exon 3a may comprise nt 257 to 269, 258 to 269, 259 to 269, 260 to 269, 261 to 269, 262 to 269, 263 to 269, 264 to 269, 265 to 269, 266 to 269 or 267 to 269 of SEQ ID NO: 6. In some embodiments, the region spans the 3′ splice site of exon 3a. The region may comprise nucleotides 1 and 2, 1 to 3, 1 to 4, 1 to 5, 1 to 6, 1 to 7, 1 to 8, 1 to 9, or 1 to 10 of SEQ ID NO: 5. Accordingly, the region in the human exon 3a may comprise nt 1 and 2, 1 to 3, 1 to 4, 1 to 5, 1 to 6, 1 to 7, 1 to 8, 1 to 9, or 1 to 10 of SEQ ID NO: 6. In some embodiments, the region comprises nt 1 and 2, 1 to 3, 1 to 4, 1 to 5, 1 to 6, 1 to 7, 1 to 8, 1 to 9, or 1 to 10 of SEQ ID NO: 6 and/or nucleotides 136 to 147, 137 to 147, 138 to 147, 139 to 147, 140 to 147, 141 to 147, 142 to 147, 143 to 147, 144 to 147, or 145 to 147 of SEQ ID NO: 32. The region may comprise nt 1 and 2, 1 to 3, 1 to 4, 1 to 5, 1 to 6, 1 to 7, 1 to 8, 1 to 9, or 1 to 10 of SEQ ID NO: 5 and/or nt 117 to 127, 118 to 127, 119 to 127, 120 to 127, 121 to 127, 122 to 127, 123 to 127, 124 to 127, 125 to 127, or 126 to 127 of SEQ ID NO: 30.

In some embodiments, the agent (e.g. ASO) targets a region within 250 nucleotides upstream or within 250 nucleotides downstream exon 3a. These upstream and downstream regions may be intron 3 regions (as shown in Table 3). The agent may target a splice enhancer element within one of the upstream or downstream regions as described in Table 3. In certain embodiments, the agent targets a region within SEQ ID NO: 30, 31, 32 or 33, preferably the region comprising a splice enhancer element.

TABLE 3
Intronic regions in RBM3 gene upstream and downstream of exon 3a.

| | Description and | |
|---|---|---|
| Genome | Position in Genome | Sequence |
| Mouse | Intron 3 upstream | GTGAGGCCTCCTGCCTGCACAGGGGCCGTCTCC |
| (GRCm39) | of exon 3a | CTGTCTGCCACTTTCTTCGTTCTTTCTGTTTTCCCT |
| | chrX: 8010811- | CTATGTCTGCCAGGGGCAGCGTGGCCACTCACA |
| | 8010685 | GGAGCATGACTGCCTTCTGTTCTAG (SEQ ID NO: 30) |
| Mouse | Intron 3 downstream | GTAAGTAGTAGCAGGGGAGACCGGTGGATTCAG |
| (GRCm39) | of exon 3a | GTCTTTTTGTGGGATTCCATAGCTCCTGGTGAAGC |
| | chrX: 8010413- | CATTCTGGTCAGCGGAGCTTGGCTTGGACGAGGC |
| | 8010187 | ACCCAGAATTTGGTGCCCAGCAGGAATTTCTGGG |
| | | CTCTTGCCTACACCTACCTGCCTGTGCCCATTCTT |
| | | CCCTCATCCCTACTGTGACTGACAATTCCTTGTGT |
| | | CTGTCTCCTTCCCATACCTAG (SEQ ID NO: 31) |
| Human | Intron 3 upstream | GTGGGGCCTCCTATCTGCAGAGGGACCTTGTCCC |
| (GRCh38) | of exon 3a | CATCTGCGGCTTCCTTCATTCTTTTTGTTTTTCCCT |
| | X chromosome, | CTGTGTCTGCTCGGGGCAGCGTGGCCACCAAGC |
| | 48575668-48575814 | CCCGCAGTGCCCACTCACAGGAGCATGACTGCCT |
| | | TTTGTTCTAG (SEQ ID NO: 32) |
| Human | Intron 3 downstream | GTAAGTAGTAGCAAGGGGAACATGGTGCATTCAG |
| (GRCh38) | of exon 3a | GTCATTTTGTGTGGGATTCTATAGCTCCTGATGAA |
| | X chromosome, | GCCATTCTGGGGTAAAGGAGCTTATTCTGGAACA |
| | 48576084-48576313 | TGGCACCCAGAACCTGGTGCCCAGCAGGAATTTT |
| | | TGGCCTCTTTTGCCCACTTACCCTTGCCTGCTCTT |
| | | CCCTCACCATTGCCCTGACTGACTGCCAACCCAT |
| | | CATCCTTGTGTCTCCCACACTTAG (SEQ ID NO: 33) |

In all embodiments herein, referring to ‘an mRNA’ or ‘the mRNA’ means one or more (at least one) mRNA molecules, and referring to ‘a pre-mRNA’ or ‘the pre-mRNA’ means one or more (at least one) pre-mRNA molecules.

ASOs are polynucleotides made of nucleotides comprising a nucleobase that may be capable of hybridising to a complementary nucleobase present on a target mRNA (e.g. pre-mRNA), a sugar moiety, and a backbone connecting the monomers. The term ASO also embodies any oligomeric molecule that comprises nucleobases capable of hybridising to a complementary nucleobase on a target mRNA (e.g. pre-mRNA), but does not comprise a sugar moiety, such as a peptide nucleic acid (PNA).

In some embodiments, an antisense oligonucleotide (ASO) is a synthetic oligonucleotide. An ASO may be an artificial, modified, non-naturally occurring oligonucleotide i.e. comprising one or more chemical modifications compared to naturally occurring RNA or DNA polynucleotides or nucleotides. Therefore the ASOs may be comprised of naturally-occurring nucleotides, nucleotide analogues, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728 and Dias and Stein, Mol. Cancer Ther. 2002, 1, 347-355, herein incorporated by reference in their entirety.

The ASO may comprise a modified nucleotide having a 2′-O-4′-C methylene bridge (LNA, locked nucleic acid) nucleotide. The ASO may comprise a RNA and/or DNA nucleotide. The ASO may include combinations of LNA nucleotides and unmodified nucleotides. The ASO may include a combination of LNA and RNA nucleotides. Antisense nucleic acids may include combinations LNA and DNA nucleotides. The combination may be alternating LNA and RNA, alternating LNA and DNA, or alternating RNA and DNA. A further preferred oligonucleotide modification includes Locked Nucleic Acids (LNAs) in which the 2′-hydroxyl group is linked to the 3′ or 4′ carbon atom of the sugar ring thereby forming a bicyclic sugar moiety.

Exemplary ASOs that target the pre-mRNA of RBM3 at splice enhancer elements or splice sites are listed in Table 4. In some embodiments, the ASO comprises one of the SEQ ID NOs listed in Table 4. In some embodiments, the ASO comprises one of SEQ ID NOs: 34, 35, 36, 37, 38, 39, 40, 41, 42, 43, 44, 45, 46, 47, 48, 49, 50, 51, 52, 53, 54, 55, 56, 57, 69 and 70, optionally wherein the sequence has one, two, or three nucleotide substitutions. In some embodiments, the ASO comprises or consists of one of SEQ ID NOs: 40, 41, 42, 43, 44, 46, 47, 48, 49, 50, 83, 84, 85, 86 and 87 optionally having one, two or three substitutions. In some embodiments, the ASO comprises or consists of one of SEQ ID NOs: 83, 84, 85 and 87, optionally having one, two or three substitutions. In certain embodiments, the ASO comprises or consists of one of SEQ ID NOs: 44, 46, 47, 48, 83, 84, 85, 86 and 87 optionally having one, two or three substitutions.

In some embodiments, the ASO comprises SEQ ID NO: 60, optionally wherein the sequence has one, two or three nucleotide substitutions.

TABLE 4
ASOs targeting splice enhancer sequences or 5′ss in mouse RBM3
pre-mRNA and the equivalent ASOs targeting the equivalent human
sequences. The mismatches between mouse and human
sequences are underlined in the equivalent human sequence.

| ASO name | SEQ ID NO | ASO Sequence (5′ to 3′) | Target in RBM3 gene |
|---|---|---|---|
| MOE | 34 | ctgctactacttacATCT | 5′ss of mouse exon |
| | | | 3a |
| hMOE | 35 | ttgctactacttacatct | 5′ss of human exon |
| | | | 3a |
| MOElongA | 36 | gtctcccctgctactacttacATCT | 5′ss of mouse exon |
| | | | 3a |
| hMOElongA | 37 | gttccccttgctactacttacatct | 5′ss of human exon |
| | | | 3a |
| MOElongB | 38 | ctgctactacttacATCTTGACTGA | 5′ss of mouse exon |
| | | | 3a |
| hMOElongB | 39 | ttgctactacttacatcttgactga | 5′ss of human exon |
| | | | 3a |
| MOE + 2 | 69 | ccctgctactacttacATC | 5′ss of mouse exon |
| | | | 3a |
| hMOE + 2 | 70 | ccttgctactacttacatc | 5′ss of human exon |
| | | | 3a |
| M2A | 40 | GTCGTCGCCTCCCAGGCAG | M2-9 of mouse exon |
| | | | 3a |
| hM2A | 41 | GTCGTCGCCTCCCAGGCGG | M2-9 of human exon |
| | | | 3a |
| M2B | 42 | GACCAGTCGTCGCCTCCCA | M2-9 of mouse or |
| | | | human exon 3a |
| M2C | 43 | TGCAGGACCAGTCGTCGCC | M2-9 of mouse or |
| | | | human exon 3a |
| M2D | 44 | CCTCATGCAGGACCAGTCG | M2-9 of mouse or |
| | | | human exon 3a |
| M2Da | 45 | TCATGCAGGACCAGTCGTC | M2-9 of mouse exon |
| | | | 3a |
| hM2Da | 46 | TCATGCAGGACCAGTCGTC | M2-9 of human exon |
| | | | 3a |
| M2Db | 47 | CGCCTCATGCAGGACCAGT | M2-9 of mouse exon |
| | | | 3a |
| hM2Db | 48 | GGCCTCATGCAGGACCAGT | M2-9 of human exon |
| | | | 3a |
| M2Dc | 83 | CTCATGCAGGACCAGTCGT | M2-9 of mouse or |
| | | | human exon 3a |
| hM2Dd | 84 | GCCTCATGCAGGACCAGTC | M2-9 of mouse or |
| | | | human exon 3a |
| hM2De | 85 | ATGCAGGACCAGTCGTCGC | M2-9 of mouse or |
| | | | human exon 3a |
| M2Df | 86 | TCCGCCTCATGCAGGACCA | M2-9 of mouse exon |
| | | | 3a |
| hM2Df | 87 | TCGGCCTCATGCAGGACCA | M2-9 of human exon |
| | | | 3a |
| M2E | 49 | CTCCGCCTCATGCAGGACC | M2-9 of mouse exon |
| | | | 3a |
| hM2E | 50 | CTCGGCCTCATGCAGGACC | M2-9 of human exon |
| | | | 3a |
| M4A | 51 | GACTCACGAGGTCCTCTCC | M4-7 of mouse or |
| | | | human exon 3a |
| M4B | 52 | CAGCAGACTCACGAGGTCC | M4-7 of mouse exon |
| | | | 3a |
| hM4B | 53 | CAGAAGACTCACGAGGTCC | M4-7 of human exon |
| | | | 3a |
| M4C | 54 | CATCCCAGCAGACTCACGA | M4-7 of mouse exon |
| | | | 3a |
| hM4C | 55 | CATGTCAGAAGACTCACGA | M4-7 of human exon |
| | | | 3a |
| M4D | 56 | GAGTCCATCCCAGCAGACT | M4-7 of mouse exon |
| | | | 3a |
| hM4D | 57 | GAGTCCATGTCAGAAGACT | M4-7 of human exon |
| | | | 3a |

*The sequence for M2D is 100% conserved between human and mouse, so SEQ ID
NOs: 45 and 46 are the same. Where ASOs are listed herein including both
SEQ ID NOs: 45 and 46, it will be appreciated that the corresponding list
without this duplication (i.e. only SEQ ID NOs: 46) is disclosed mutatis
mutandis.

The present invention provides an antisense oligonucleotide comprising SEQ ID NO: 34, 35, 36, 37, 38, 39, 40, 41, 42, 43, 44, 45, 46, 47, 48, 49, 50, 51, 52, 53, 54, 55, 56 or 57, for use in a method of treating or preventing a disease affected by RBM3 expression in a subject. The ASO may comprise SEQ ID NO: 34, 35, 36, 37, 38, 39, 40, 41, 42, 43, 44, 45, 46, 47, 48, 49, 50, 51, 52, 53, 54, 55, 56 or 57 with one, two or three nucleotide substitutions. In some embodiments, the ASO comprises SEQ ID NO: 60, optionally wherein the sequence has one, two or three nucleotide substitutions.

The present invention provides an antisense oligonucleotide comprising SEQ ID NO: 34, 35, 36, 37, 38, 39, 40, 41, 42, 43, 44, 46, 47, 48, 49, 50, 51, 52, 53, 54, 55, 56, 57, 83, 84, 85, 86 or 87 for use in a method of treating or preventing a disease affected by RBM3 expression in a subject.

The ASO may comprise SEQ ID NO: 34, 35, 36, 37, 38, 39, 40, 41, 42, 43, 44, 46, 47, 48, 49, 50, 51, 52, 53, 54, 55, 56, 57, 83, 84, 85, 86 or 87 with one, two or three nucleotide substitutions.

In some embodiments, the ASO for use comprises or consists of one of SEQ ID NOs: 40, 41, 42, 43, 44, 46, 47, 48, 49, 50, 83, 84, 85, 86 and 87.

In some embodiments, the ASO for use comprises or consists of one of SEQ ID NOs: 83, 84, 85 and 87.

In certain embodiments, the ASO for use comprises or consists of one of SEQ ID NOs: 44, 46, 47, 48, 83, 84, 85, 86 and 87.

Any of the ASOs described herein may contain a sugar moiety that comprises ribose or deoxyribose, as present in naturally occurring nucleotides, or a modified sugar moiety, including sugar moieties containing a morpholine ring. Non-limiting examples of modified sugar moieties include 2′ substitutions such as 2′-O-methyl, 2′-O-methoxyethyl (MOE), 2′-O-aminoethyl, 2′-fluoro (2′F); N3′->P5′ phosphoramidate, 2′dimethylaminooxyethoxy, 2′dimethylaminoethoxyethoxy, 2′-guanidinium, 2′-O-guanidinium ethyl, carbamate modified sugars, and bicyclic modified sugars. In some embodiments, the sugar moiety modification is selected from as 2′-O-methyl, 2′-fluoro and 2′-O-methoxyethyl (MOE).

The ASOs described herein also comprise a backbone structure that connects the components of an oligomer. The term “backbone structure” and “oligonucleotide linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligonucleotide linkages of the ASO described herein may include (but are not limited to) phosphorothioate, phosphorodithioate, phosphoroselerloate, phosphorodiselenoate, phosphoroanilothioate, phoshoraniladate, phosphoronmidate, and the like. In some embodiments, the backbone modification is a phosphorothioate linkage. See e.g., LaPlanche et al. Nucleic Acids Res. 14:9081 (1986); Stec et al. J. Am. Chem. Soc. 106:6077 (1984), Stein et al. Nucleic Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990).

In some examples, each monomer of the ASO is unmodified or is modified in the same way, for example each linkage of the backbone of the ASO comprises a phosphorothioate linkage or each ribose sugar moiety comprises a 2′-O-methoxyethyl (MOE) modification. Such modifications that are present on each of the monomer components of an ASO are referred to as “uniform modifications” and the ASO is referred to as “fully modified”. In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries”.

In some embodiments, the ASO comprises one or more backbone modifications. In some embodiments, the ASO comprises one or more sugar moiety modification. In some embodiments, the ASO comprises one or more backbone modification and one or more sugar moiety modification. In some embodiments, the ASO comprises MOE modifications and a phosphorothioate backbone. In some embodiments, the ASO comprises 2′-O-methyl modifications and a phosphorothioate backbone. In some embodiments, the ASO comprises a phosphorodiamidate morpholino (PMO). In some embodiments, the ASO comprises a peptide nucleic acid (PNA). In some embodiments, the ASO comprises a ribofuranosyl or 2′deoxyribofuranosyl modification. In some embodiments, the ASO comprises 2′4′-constrained 2′O-methyloxyethyl (cMOE) modifications. In some embodiments, the ASO comprises cEt 2′, 4′-constrained 2′-O ethyl BNA modifications.

A phosphate backbone of the ASO can be modified to generate peptide nucleic acid molecules. As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols, for example.

Antisense nucleic acids can also be formulated as morpholino oligonucleotides. In such embodiments, the riboside moiety of each subunit of an oligonucleotide of the oligonucleotide reagent is converted to a morpholine moiety. Morpholinos may also be modified, e.g. as a peptide conjugated morpholino, a phosphorodiamidate morpholino, etc.

In other embodiments, the antisense oligonucleotide can be linked to functional groups such as peptides (e.g., for targeting host cell receptors in vivo), or agents facilitating transport across the cell membrane or the blood-brain barrier. Oligonucleotide reagents of the disclosure also may be modified with chemical moieties (e.g., cholesterol) that improve the in vivo pharmacological properties of the oligonucleotide reagents. Oligonucleotides of the disclosure may also be formed as a DNA/RNA heteroduplex oligonucleotide (HDOs) as described in Nagata et al. Nat Biotechnol (2021) https://doi.org/10.1038/s41587-021-00972-x, herein incorporated by reference in its entirety. Conjugation to cholesterol or α-tocopherol at the 5′ end of the RNA strand has been shown to allow the HDO to reach the CNS after subcutaneous or intravenous administration (Nagata et al. 2021).

Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more component of any ASO may be modified to enhance binding affinity to a target sequence on a mRNA (e.g. pre-mRNA); reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and modulate the half-life of the ASO.

In some embodiments, the ASOs are comprised of MOE-phosphorothioate-modified nucleotides. ASOs comprised of such nucleotides are especially well-suited to the methods disclosed herein; oligonucleotides having such modifications have been shown to have significantly enhanced resistance to nuclease degradation and increased bioavailability, making them suitable, for example, for oral, intrathecal or systemic delivery in some embodiments described herein. See e.g., the FDA approved drug nusinersen²² which is well distributed throughout the central nervous system after intrathecal injection²³.

Methods of synthesising oligonucleotides such as ASOs are known to one of skill in the art. Alternatively or in addition, ASOs may be obtained from a commercial source.

### Other Methods

In another aspect, a method of identifying an antisense oligonucleotide (ASO) capable of increasing expression of RBM3 in a cell is provided. The method comprises:
i) identifying an ASO that targets a region of the pre-mRNA of the RBM3 gene, within a region selected from: a region within exon 3a, a region spanning a splice site of exon 3a, a region located within 250 nucleotides upstream exon 3a and a region located within 250 nucleotides downstream exon 3a, or a region as described herein,
ii) delivering the ASO identified in step i) to the cell, and
iii) measuring the level of expression of RBM3 in the cell of step ii). Optionally, the method further comprises:
iv) comparing the level of expression measured in step iii) with the level of expression of RBM3 in a cell treated with a control ASO or DMSO. The level of expression of RBM3 may be the transcript level or protein level. The level of expression may be measured by RT-qPCR or western blotting.

Unless specified otherwise, the left-hand end of single-stranded nucleic acid (e.g., mRNA, oligonucleotide, ASO etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, an initiation or start codon is located near the 5′ end and the termination codon is located near the 3′ end.

In certain aspects, a method for inhibiting non-sense mediated decay of RBM3-encoding mature mRNA in a cell, a method for increasing/inducing expression of RBM3 in a cell, and a method for modulating splicing of a RBM3-encoding pre-mRNA in a cell are provided. Each method may comprise exposing the cell to an agent, wherein the agent is capable of hybridising to a region of the pre-mRNA of RBM3 such as to alter the splicing of the pre-mRNA such that in the resulting mature mRNA exon 3a is not incorporated, as described herein.

The method for inhibiting non-sense mediated decay of RBM3-encoding mature mRNA in a cell, the method for increasing/inducing expression of RBM3 in a cell, and the method for modulating splicing of RBM3-encoding pre-mRNA in a cell as described herein aim to increase the level of expression of RBM3. In some embodiments, the method is performed in vitro. In certain embodiments, the method is performed without cooling, preferably the method is performed at a temperature of ≥34° C., ≥35° C., ≥36° C., or ≥37° C. In other embodiments, each of these methods is performed with cooling, preferably the method is performed at a temperature ≤35° C., ≤34° C., ≤33° C. or ≤32° C. In some embodiments, the cell is a cell of the central nervous system. In some embodiments, the cell is a neuron, preferably a primary neuron and more preferably a primary hippocampal neuron. In some embodiments, the cell is an astrocyte, oligodendrocyte, microglial cell, ependymal cell or brain stem cell. In certain embodiments, the cell is a mouse or human cell. In some embodiments, the cell is in vitro or ex vivo.

As used herein, ‘expression’ may be gene expression or protein expression and thus may be measured by quantifying levels of a transcript of RBM3 or the protein levels of RBM3.

Gene expression can be determined e.g. by detection of mRNA encoding RBM3. Methods for measuring or quantifying mRNA levels are well known in the art, and include, for example, RT-PCR, quantitative real-time PCR (RT-qPCR), microarray analysis, northern blot analysis, RNase protection analysis, or any other suitable method, for example as described in Rio, D. C., RNA: A Laboratory Manual, Cold Spring Harbor Laboratory Press, 2011 (incorporated herein in its entirety).

Protein expression can be determined e.g. by detection of the protein, for example by antibody-based methods which are well known to the skilled person, such as western blot analysis, immunohistochemistry, immunocytochemistry, flow cytometry, ELISA, and mass spectrometry, or any other suitable method, for example as described in Link, A. J., Proteomics: A Cold Spring Harbor Laboratory Course Manual, Cold Spring Harbor Laboratory Press, 2009 (incorporated herein in its entirety).

As used herein, the term “inhibiting NMD” means reducing (partially or completely) the extent to which NMD occurs. Inhibition of NMD results in an increase in levels of mRNA of RBM3. For instance, inhibiting NMD of RBM3-encoding mature mRNA results in a measurable increase in the amount of mRNA of RBM3. As used herein, “mRNA of RBM3” or “RBM3 mRNA” includes all mRNA isoforms (transcripts) of the RBM3 gene and the levels of mRNA of RBM3 may be measured using RBM3-specific PCR primers that are not splicing-sensitive. An example of such a pair of primers is given in Table 6 of Example 5.

Inhibition of NMD results in an increase in RBM3 mRNA levels of 5% or more, such as 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 550%, 600%, 650%, 700%, 750%, 800%, 850%, 900%, 1000% or more, compared to RBM3 mRNA levels in the absence of the agent or ASO/absence of treatment (e.g., total levels wherein no agent or ASO is present in the cell), or compared to RBM3 mRNA levels when treatment with a control ASO occurs (e.g. total levels wherein a control ASO is present in the cell). Inhibition of NMD results in an increase of 1.01-, 1.05-, 1.10-, 1.25-, 1.50-, 2.0-, 2.5-, 3.0-, 3.5-, 4.0-, 4.5-, 5.0-, 5.5-, 6.0-, 6.5-, 7.0-, 7.5-, 8.0-, 8.5-, 9.0-, 9.5-, 10-fold or more in RBM3 mRNA levels as compared to RBM3 mRNA levels in the absence of the agent or ASO/absence of treatment (e.g., total levels wherein no agent or ASO is present in the cell) or compared to RBM3 mRNA levels when treatment with a control ASO occurs (e.g. total levels wherein a control ASO is present in the cell).

Inhibition of NMD of RBM3-encoding mature mRNA through inhibiting inclusion of exon 3a leads to an increase in levels of the mRNA isoform that does not contain exon 3a, i.e. mature mRNA wherein exon 3a is not incorporated (exon 3a is skipped) or mature mRNA wherein exons 3 and 4 are spliced together without an intervening exon. The levels of such mRNA may be measured using splicing-sensitive PCR primers. Inhibition of NMD through inhibiting inclusion of exon 3a results in an increase in levels of such mRNA of 5% or more, such as 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 550%, 600%, 650%, 700%, 750%, 800%, 850%, 900%, 1000% or more, compared to the levels in the absence of the agent or ASO/absence of treatment (e.g., total levels wherein no agent or ASO is present in the cell), or compared to levels when treatment with a control ASO occurs (e.g. total levels wherein a control ASO is present in the cell). Inhibition of NMD results in an increase of 1.01-, 1.05-, 1.10-, 1.25-, 1.50-, 2.0-, 2.5-, 3.0-, 3.5-, 4.0-, 4.5-, 5.0-, 5.5-, 6.0-, 6.5-, 7.0-, 7.5-, 8.0-, 8.5-, 9.0-, 9.5-, 10-fold or more in levels of such mRNA as compared to the levels in the absence of the agent or ASO/absence of treatment (e.g., total levels wherein no ASO is present in the cell) or compared to the levels when treatment with a control ASO occurs (e.g. total levels wherein a control ASO is present in the cell).

Inhibition of NMD through inhibiting inclusion of exon 3a results in a decrease in the inclusion frequency of exon 3a. The inclusion frequency of exon 3a may be quantified as ‘Percent Spliced In’ (PSI) which corresponds to the ratio of the abundance of the isoform of mRNA in which exon 3a is incorporated over the abundance of all of the isoforms of RBM3 (×100%). A difference in PSI between two conditions is termed ‘dPSI’ and is calculated by subtraction (dPSI (%)=PSI_{control}−PSIₜᵣₑₐₜₘₑₙₜ). An inhibitor of translation may be used to stabilise the exon 3a-containing mRNA thus allowing the accumulation and detection of exon 3a-containing mRNA. An exemplary inhibitor of translation is cycloheximide (CHX). Inhibition of NMD through inhibiting inclusion of exon 3a results in a decrease in the inclusion frequency (PSI) of exon 3a, wherein the decrease is 1% or more, such as 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, as compared to the inclusion level in the absence of the agent or ASO/absence of treatment (e.g., PSI wherein no agent or ASO is present in the cell), or compared to the inclusion level (PSI) wherein a control ASO is present in the cell.

Inhibition of NMD through inhibiting inclusion of exon 3a results in an increase of the level of protein for example the level of a protein product of a transcript of RBM3. For instance, inhibiting NMD of RBM3-encoding mature mRNA through inhibiting inclusion of exon 3a results in a measurable increase in the amount of total protein translated from the RBM3 mRNA. The protein product may be a truncated or full-length protein. In some aspects, inhibition of NMD through inhibiting inclusion of exon 3a results in an increase in RBM3 protein levels of 5% or more, such as 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500% or more, compared to RBM3 protein levels in the absence of the agent or ASO/absence of treatment (e.g., levels of expression of RBM3 protein wherein no ASO is present in the cell) or RBM3 protein levels when treatment with a control ASO occurs. In some aspects, inhibition of NMD through inhibiting inclusion of exon 3a results in a 1.01-, 1.05-, 1.10-, 1.20-, 1.25-, 1.3-, 1.35-, 1.40-, 1.45-1.50-, 1.55-, 1.60-, 1.65-, 1.70-, 1.75-, 1.80-, 1.85-, 1.90-, 1.95-2.0-, 2.5-, 3.0-, 3.5-, 4.0-, 4.5-, 5.0- or more fold increase in protein levels, as compared to the RBM3 protein levels in the absence of the agent or ASO/absence of treatment or the RBM3 protein levels in the presence of a control ASO.

### Methods of Administration

ASOs can be provided to an individual by any of a variety of methods, such as those described by Juliano et al. referenced above, such as delivery of “free” or “naked” ASOs which are taken up by some cells, delivery of ASOs conjugated to cell penetrating peptides (CPPs, e.g., TAT and Antennapedia peptides), delivery of ASOs conjugated to ligands for cell receptor uptake (e.g., ASO-cholesterol conjugates, ASO-folate conjugates, N-acetyl galactosamine conjugates, ASO-insulin-like growth factor 1 conjugates, ASO-RGD peptide conjugates, ASO-bombesin conjugates, etc.), delivery of ASOs associated with nanocarriers (e.g., lipid-based carriers,

ASOs associated with perfluorocarbon nanoparticles, ASO-antibody conjugates, etc.). Other methods for delivery of nucleic acids such as ASOs are known in the art, and include, but are not limited to, forming nucleic acid conjugates with cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al, EMBO J., 1991, 10, 1111-1118; Kabanov et al, FEBS Lett, 1990, 259, 327-330; Svinarchuk et al, Biochimie, 1993, 75, 49-54), a phospholipid, e.g, di-hexadecyl-racglycerol or triethylammonium 1,2-di-O-hexadecyl-racglycero-3-H-phosphonate (Manoharan et al. Tetrahedron Lett., 1995, 36, 3651-3654; Shea et al, Nucl. Acids Res., 1990, 18, 3777-3783), a polyamine or a polyethylene glycol chain (Manoharan et al. Nucleosides & Nucleotides, 1995, 14, 969-973), or adamantane acetic acid (Manoharan et al. Tetrahedron Lett., 1995, 36, 3651-3654), a palmityl moiety (Mishra et al, Biochim. Biophys. Acta, 1995, 1264, 229-237), or an octadecylamine or hexylamino-carbonyl-oxycholesterol moiety (Crooke et al, J. Pharmacol. Exp. Ther., 1996, 277, 923-937.

### Diseases or Conditions

The methods and agents of the present invention can be useful in the treatment or prevention of any disease that with the potential to be affected by RBM3 expression. Accordingly, the present invention provides an agent as described herein for use in a method of treating or preventing a disease affected by RBM3 expression in a subject.

Since therapeutic hypothermia is known to induce expression of RBM3, the methods and agents of the present invention may be useful in the treatment or prevention of any disease or condition that is treated or prevented by inducing therapeutic hypothermia.

Therapeutic hypothermia is one of the most robust neuroprotectants studied to date (Yenari M. & Han H., Neuroprotective mechanisms of hypothermia in brain ischaemia. Nature Reviews Neuroscience, 13, 267-278 (2012)). Therefore, in some embodiments, the disease is a neurodegenerative disease or neurological damage or injury. In some embodiments, the disease is depression or anxiety. In some embodiments, an agent of the invention is used as a neuroprotectant. For example, the agents of the invention may be used as a neuroprotectant during cardiac surgery or induced coma or in the treatment of hypoxic ischemic encephalopathy in neonates, or may be used as a neuroprotectant to treat or prevent a disease or condition described herein. A neuroprotectant is an agent that is capable of increasing neuroprotection.

In some embodiments, the disease is a neurodegenerative disease. In certain embodiments, the disease is Alzheimer's disease, prion disease, Parkinson's disease, frontotemporal dementia, amyotrophic lateral sclerosis, a tauopathy, amyotrophic lateral sclerosis (ALS), vascular dementia and related disorders.

RBM3 induction or over-expression is protective in animal models of prion disease and Alzheimer's disease (Peretti et al; Nature 518, pages 236-239 (2015), Peretti et al; Life Science Alliance vol. 4 no. 4 e202000884 (2021).

In some embodiments, the disease is neurological damage or injury. In certain embodiments, the disease is stroke, head or brain injury, spinal cord injury, neonatal hypoxic ischemic encephalopathy. In some embodiments, the disease is anoxic brain injury. In some embodiments, the disease is neurological damage caused by cardiac arrest. In certain embodiments, the disease is neurological damage caused during cardiac surgery or induced coma.

The following sections provide further description relating to diseases/conditions which are affected by RBM3 and/or induced hypothermia.

Therapeutic hypothermia can not only efficiently reduce primary injury and prevent secondary injury in acute ischemia (Yenari M & Han H, 2012) and spinal cord injury (SCI) (Alkabie S, Boileau A J (2015) The role of therapeutic hypothermia after traumatic spinal cord injury—a systematic review. World Neurosurg. doi:10.1016/j.wneu.2015.1009.1079), but also delay the progression of chronic neurodegenerative diseases (Salerian A J, Saleri N G (2008) Cooling core body temperature may slow down neurodegeneration. CNS Spectr 13(3):227-229). In mouse models, hypothermia and induction of RBM3 by cooling, by inducing its upstream or downstream mediators is profoundly neuroprotective in prion-diseased mice and Alzheimer's mice, restoring synapse number, memory, preventing brain cell death and increasing survival (Peretti et. al. Nature 2015; Bastide et al., 2017, Current Biology 27, 638-650; Peretti et al. Life Sci Alliance. 2021; 4(4):e202000884. doi: 10.26508/lsa.202000884). In vitro, the two cold-inducible proteins CIRP and RBM3 both function against apoptosis in cultured primary neurons or neuron-like PC12 cells [Chip S, et al. (2011) Neurobiol Dis 43(2):388-396; Zhang H T, et al. (2015) Brain Res 1622:474-483; Kita H, et al. (2002) Hum Mol Genet 11(19):2279-2287; Zhu et al. Cell. Mol. Life Sci. (2016) 73:3839-3859).

Yenari M & Han H (2012) discuss that clinical studies have established a role for therapeutic hypothermia in neuroprotection in some clinical conditions, including anoxic brain injury due to cardiac arrest (Bernard, S. A. et al. Treatment of comatose survivors of out-of-hospital cardiac arrest with induced hypothermia. N. Engl. J. Med. 346, 557-563 (2002); The Hypothermia after Cardiac Arrest Study Group. Mild therapeutic hypothermia to improve the neurologic outcome after cardiac arrest. N. Engl. J. Med. 346, 549-556 (2002)) and hypoxic ischaemic neonatal encephalopathy (Gluckman, P. D. et al. Selective head cooling with mild systemic hypothermia after neonatal encephalopathy: multicentre randomised trial. Lancet 365, 663-670 (2005); Shankaran, S. et al. Whole-body hypothermia for neonates with hypoxic-ischemic encephalopathy. N. Engl. J. Med. 353, 1574-1584 (2005)).

The above-mentioned studies establish a clear role of RBM3 induced through therapeutic hypothermia, experimental cooling or over-expression in mouse models in treating and preventing spinal cord injury, neurodegenerative disease, anoxic brain injury and hypoxic ischaemic neonatal encephalopathy.

In addition to the neuroprotective effect, high RBM3 levels have been clinically associated with prolonged overall survival in cancer (Avila-Gomez P. et al., Cold stress protein RBM3 responds to hypothermia and is associated with good stroke outcome, Brain Communications, Volume 2, Issue 2, 2020, https://doi.org/10.1093/braincomms/fcaa078). For example, prolonged overall survival was observed in patients having intestinal-type gastric cancer (Ye F P et al. Med Sci Monit 2017; 23:6033-41), invasive breast cancer (Kang et al., J Breast Cancer 2018; 21:288-96) and colon cancer (Jang H H et al., Anticancer Res 2017; 37:1779-85), and in metastatic colorectal cancer (Siesing C, et al. PLoS One 2017; 12: e0182512.).

In some embodiments, the disease is cancer. In certain embodiments, the cancer is gastric cancer, breast cancer, colon cancer or colorectal cancer.

The present invention provides methods and compositions for the treatment or prevention of a disease or a condition.

The term “treatment,” as used herein in the context of treating a condition, pertains generally to treatment and therapy of a human subject, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

In some embodiments, the method further involves contacting, introducing, delivering, or administering to the subject two agents or two ASOs as described herein.

Administration of an agent (e.g. ASO) according to the present invention is preferably in a “therapeutically effective” or “prophylactically effective” amount, this being sufficient to show benefit to the subject.

The term “therapeutically-effective amount” as used herein, pertains to that amount of the agent (e.g. ASO) which is effective for producing some desired therapeutic effect, commensurate with a reasonable benefit/risk ratio, when administered in accordance with a desired treatment regimen.

Similarly, the term “prophylactically effective amount,” as used herein pertains to that amount of the agent (e.g. ASO) which is effective for producing some desired prophylactic effect, commensurate with a reasonable benefit/risk ratio, when administered in accordance with a desired treatment regimen.

“Prophylaxis” in the context of the present specification should not be understood to describe complete success i.e. complete protection or complete prevention. Rather prophylaxis in the present context refers to a measure which is administered in advance of detection of a symptomatic condition with the aim of preserving health by helping to delay, mitigate or avoid that particular condition.

In therapeutic applications, agents of the present invention are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The invention further provides a composition comprising an agent (e.g. ASO, vector) as described herein. The composition is preferably a pharmaceutical composition or medicament.

Any appropriate antisense nucleic acid disclosed herein may be administered to a subject. For example, the antisense nucleic acid may be an ASO as described herein. In some embodiments, an ASO is expressed from a transgene, e.g., as an antisense RNA transcript. A transgene may be administered to a subject in a DNA expression construct that is engineered to express an antisense RNA transcript in a subject. A DNA expression construct may be administered directly or using a viral vector (e.g., a recombinant AAV (rAAV) vector) or other suitable vector. Viral vectors that have been used for gene therapy protocols include, but are not limited to, retroviruses, other RNA viruses such as poliovirus or Sindbis virus, adenovirus, adeno-associated virus (AAV), herpes viruses, SV 40, vaccinia, lentivirus and other DNA viruses. Alternatively, a transgene may be express ex vivo and the resulting antisense RNA transcript may be administered directly to the subject.

Any appropriate polynucleotide (e.g. the polynucleotide encoding the CRISPR/Cas-based base editing system) disclosed herein may be administered to a subject. For example, as described herein. In some embodiments, an CRISPR/Cas-based base editing system is expressed from one or more transgenes which may be administered to a subject in a DNA expression construct that is engineered to express the CRISPR/Cas-based base editing system in a subject. A DNA expression construct may be administered directly or using a viral vector (e.g., a recombinant AAV (rAAV) vector) or other suitable vector. Viral vectors that have been used for gene therapy protocols include, but are not limited to, retroviruses, other RNA viruses such as poliovirus or Sindbis virus, adenovirus, adeno-associated virus (AAV), herpes viruses, SV 40, vaccinia, lentivirus and other DNA viruses.

As disclosed herein antisense nucleic acids (including DNA expression constructs that may be used to expressed them) may be administered by any suitable route. For use in therapy, an effective amount of the antisense nucleic acid (e.g. oligonucleotide) and/or other therapeutic agent can be administered to a subject by any mode that delivers the agent to the desired tissue. In some embodiments, agents (e.g., ASOs) are administered intrathecally or systemically. Other suitable routes of administration include but are not limited to oral, parenteral, intramuscular, intravenous, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulisers.

For oral administration, the agents can be formulated readily by combining the active compound(s) with pharmaceutically acceptable carriers well known in the art. Such carriers enable the agents of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally oral formulations may also be formulated in saline or buffers for neutralising internal acid conditions or may be administered without any carriers.

Pharmaceutical preparations that can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. Formulations for oral administration are typically in dosages suitable for such administration.

For buccal administration, the compositions may take the form of tablets or lozenges formulated in conventional manner.

For administration by inhalation, agents (e.g., antisense nucleic acids) for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The agents (e.g., antisense nucleic acids), when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilising and/or dispersing agents.

### Subjects

Subjects may be animal or human. Subjects are preferably mammalian, more preferably human. The subject may be a non-human mammal, but is more preferably human. The subject may be male or female. The subject may be a patient. The patient may have a disease/condition as described herein. A subject may have been diagnosed with a disease/condition requiring treatment, may be suspected of having such a disease/condition, or may be at risk from developing such disease/condition.

### Sequences

TABLE 5
Sequences

| Description | SEQ ID NO | Sequence |
|---|---|---|
| RBM3 | 58 | ATCAATCGTCTTCCGGCGCAGCCCCGTCCCTGTTTTTTGTGCTCC |
| mature | | TCCGAGCTCGCTGTTCGTCCGGGTT |
| mRNA | | TTTTACGTTTTAATTTCCAGGACTTGAACTGCCATGTCCTCTGAAG |
| (Human) | | AAGGAAAGCTCTTCGTGGGAGGGC |
| (NM)006743.5 | | TCAACTTTAACACCGACGAGCAGGCACTGGAAGACCACTTCAGC |
| | | AGTTTCGGACCTATCTCTGAGGTGGT |
| | | CGTTGTCAAGGACCGGGAGACTCAGCGGTCCAGGGGTTTTGGTT |
| | | TCATCACCTTCACCAACCCAGAGCAT |
| | | GCTTCAGTTGCCATGAGAGCCATGAACGGAGAGTCTCTGGATGG |
| | | TCGTCAGATCCGTGTGGATCATGCAG |
| | | GCAAGTCTGCTCGGGGAACCAGAGGAGGTGGCTTTGGGGCCCA |
| | | TGGGCGTGGTCGCAGCTACTCTAGAGG |
| | | TGGTGGGGACCAGGGCTATGGGAGTGGCAGGTATTATGACAGT |
| | | CGACCTGGAGGGTATGGATATGGATAT |
| | | GGACGTTCCAGAGACTATAATGGCAGAAACCAGGGTGGTTATGA |
| | | CCGCTACTCAGGAGGAAATTACAGAG |
| | | ACAATTATGACAACTGAAATGAGACATGCACATAATATAGATACA |
| | | CAAGGAATAATTTCTGATCCAGGAT |
| | | CGTCCTTCCAAATGGCTGTATTTATAAAGGTTTTTGGAGCTGCAC |
| | | CGAAGCATCTTATTTTATAGTATAT |
| | | CAACCTTTTGTTTTTAAATTGACCTGCCAAGGTAGCTGAAGACCT |
| | | TTTAGACAGTTCCATCTTTTTTTTT |
| | | AAATTTTTTCTGCCTATTTAAAGACAAATTATGGGACGTTTGTAGA |
| | | ACCTGAGTATTTTTCTTTTTACCA |
| | | GTTTTTTAGTTTGAGCTCTTAGGTTTATTGGAGCTAGCAATAATTG |
| | | GTTCTGGCAAGTTTGGCCAGACTG |
| | | ACTTCAAAAAATTAATGTGTATCCAGGGACATTTTAAAAACCTGTA |
| | | CACAGTGTTTATTGTGGTTAGGAA |
| | | GCAATTTCCCAATGTACCTATAAGAAATGTGCATCAAGCCAGCCT |
| | | GACCAACATGGTGAAACCCCATCTG |
| | | TACTAAACATAAAAAAATTAGCCTGGCATGGTGGTGTACGCCTGT |
| | | AATCCCAGTGACTTGGGAGGCTGAG |
| | | GCAGGAGAATCGCTTGAACCCGGGAGGCGGAGGTTGCAGTGAG |
| | | CTAAGATCGCGCCACTGTACTCCAGCC |
| | | TGGGCAACAGCGAGACTCCATCTCAAAAAAAAAGGAAATGTGTAT |
| | | CAAGAACATGATTATCCAGCGGTAT |
| | | TTTCTAATTCAGATCATCAAACTGATTATATAGAAGAGTTGGCTTT |
| | | AAAATGTTTGCAAATGTCTTTTTT |
| | | TTTTTAATACTGGAAGAAAAAATATTCTGTTGTGTCTCATACAGTG |
| | | CTTAGGATGTCTTTCACAGAGCTT |
| | | ATTAAAAAGATGAAACCTGAGAACAAACTGCTTTATTCTTACTCAG |
| | | CCCATTTTGCAAATTAAAAGTGGG |
| | | GGCAGAGGTGGGCGGATCACCTGAGGTCAGGAGTTCGAGACCA |
| | | GCCTGGCCAACAGGGCAAAACCCCATC |
| | | TCTACTAAAAATACAAAAGTTAGCGGGGCGTGGTGGCGGGTGCA |
| | | TGTAATCCCAGCTACTCGGGAGGCTG |
| | | AGGCAGGAGAATTGCTTGAACCCAGGAGGCGGAAGTTGCAGTG |
| | | AGCTGAGATTGTGCCACTGCACTCCAG |
| | | CCTAGGTGACAGCAAGACTCTGTCTCAAAAAAAAAAAAATGGCAC |
| | | ATCAACGAGAGGGAGGCTTGGAGAA |
| | | TATTTGGTTGGTGGGTGGGCAGGGACTGCCAGAGGGTTAGGTGT |
| | | ACATTGAGGCCTGAGGCCTGCTGGAA |
| | | TTGGGTTTCCTTAACTGGTCTCTTATCTTAGTCCCATTGTTCTGAG |
| | | AACATAAGCACCTAATCTCATGGC |
| | | GTGAGCTAATCCCTATCATAAATAGTGGTACCAGTTCATTCCTTC |
| | | CCCTGAAATGATGGGGGTTGGGCAG |
| | | AGATGCACTGAACTCATCTTTGTGTATGAGGGCCATGAGAGAGG |
| | | CCTGGTTTGAAAACAATGCTAAGAGC |
| | | TCAGATTCCAGGATTTATGTAGGGGCTCTTCTACTTCCCAGGTAT |
| | | TTGACCTCAGGTGAGTCATTTAATT |
| | | TCAGCCTTATGCCATTTTACATTTTCTGTGGAGGGTTAGGTTAATT |
| | | GGCATATAAAGCCACTGAGAAGAG |
| | | TTGGCAAAGCTCTCAGTCACGGGGATTTGATCTTTTTTGGTGTGT |
| | | GTGTGTCTTGGGTGGTAAAGTGGTA |
| | | ATTTTGTAATTCTAGCTGGTCTTAGTTCCTCAGTTCTGCTCCCTTT |
| | | AGTCATGGTTCTTTCTAGTGGCTG |
| | | TATTGAGGCCCCATGGGTGTTATCCCTCCATTGGTTCTAGTTTGG |
| | | AACAGAAAAATCTGTTGTATTCATG |
| | | GCTTTCACCTGGCTAATACTGAGCTAAGTTACCACCAGGTTGCAA |
| | | ACTCCAGGACATTATTGTCCTGAGC |
| | | TGCCTATTCCCTTGCTGGTGCGTTGTGGAACCCTGTATTATTAGT |
| | | TCCAGTCCTGGAGGCCTGCCTCCTG |
| | | AGTTTCCCAGCTAGTTGGGACAGGCCCCAATATCCCTTCTTCCTG |
| | | TAACCCAAACAGTCATGAATTTGCT |
| | | TTGGCAGGTGGATGGAGACTTGGGAACTTCCACCTCACCCACTA |
| | | AGCCAGGCCCAGGCTATGGGGCATTG |
| | | TGGCTAACCCCACCAGGTGGATACTTGGTCTGAGGACGCATCTT |
| | | ATTCTGGGCCTTTAGGGAGCTAAGGC |
| | | AGTGAGAATTGGCAGGAGGACTGCTGTGAATGCCTTGTAGGTCG |
| | | GGGGATTGGAGGGGGTCCTCTGCTCC |
| | | CTGTCTCTCAGGATGGCAATGTACCTCTCACCCCCAGTGTCAGC |
| | | TGAGGTAGGATCCATAATCAGTAATA |
| | | TTCCTGCAACAAAATGTTCCTAAGTGGAATCAATGAAGACCAAAA |
| | | TTTATTTGTATAACAACTCTAAACC |
| | | TGCTCTGCTCTGCTGTGTTTACAATGTGCTTTGTGATCATCCAGC |
| | | CCAGGGAGTCCCAGTCTCTGGCTGT |
| | | CTATCTGCTCTAAGGAAGAGAGCCCACAGTTCTCTATAGTGCCAT |
| | | AGTCTGTGATGAATAAAGTTCCAGA |
| | | TTTGAGGTCAACCCCCGACCACCCTTAAAGTGCTTGTTGGTCTCC |
| | | TACTTTGGTTTGTCTTCAGCATCCA |
| | | ACTGATGCACATTTGTCAAGAACCCACTGAGGCTAGAAACCCCC |
| | | CAAACACTCAAGGTACCTTGAGAACA |
| | | TGTATCCCAAGACCTGCAAAGACCAAACACATGGCCTGTAACTAC |
| | | TTTTCACTTTCAAGCCCTGGAACTT |
| | | GTCTGTACCAAAAATCTTTCTGAACCATTCACCAAATCTCTTGTCA |
| | | AGGGCCAGAATAAATTAAGAGATT |
| | | GGCTTTTGGTTTTCAATTAGGCTTTCAAGTCCTAAGCCCAACCCC |
| | | TCCTATCCCACCTAGGCAGAAATCA |
| | | CTTCAGATCCTTGCTTCCTAGTTTAATTTCTTCTCTGCCTCAGTTA |
| | | TAACATCCATAAAATGGAAATAAT |
| | | AATACCCACCTCACACAGGGGTTAGGAGTGTTTTTTTGTTGGTTT |
| | | TTTTCCAGACAGGGTCTCACTCTGT |
| | | CTCCCAGGCTGGAGTGCAGTGGCACAATCTCGGCTCACTGTACC |
| | | CTCCGCCTCCCAGGTTCAAGCAGTTC |
| | | TCATGCCTCAGCCTCCCAAGTAGCTGGGATTACAGGTGCCTGCC |
| | | ACCACGCCCGGCTAATTTTTGTATTA |
| | | ATAGAGACAGGGTTTCACCGTGTTGGGCAGGCTGGTCCTGAACT |
| | | CCTGACCTCAAGTGATCCACCCGCCT |
| | | CGGCCTCCCAAAGTGCTGGGATTACAGGCGTGAGCCACCACAC |
| | | CTGGCCTAGGAGTTAAGAGTATTAAAT |
| | | GTTAAGAACAGACCAAGTCCATCAAACAGCTCCACACCCTTGTTC |
| | | CATGTTCTGGATTGGGGAGTTGTGG |
| | | GGGTGGAGGTGTAGAACTTTTAAAAAGCTTCTTAAAATAAGTTGC |
| | | TGTGAATACTTCAGGTATATAAAAA |
| | | AACATTGGTTGACAATAAGCCAGTGTTCTGCCATTCTTACCTGCT |
| | | TATCAAGACAAAACCTGCTCAAGTC |
| | | CCTGCCCAGCTGCATTCCAAGTGTTTTCAAGTTTGGTGGTAAGAC |
| | | CTGACCTGAGGCTTCTTATAACCTT |
| | | TACTCAGTGGGAATATGCATACAGTTCACTACAGAAATATTTGTAT |
| | | TTACTTAGAGGAAGTGCCCTGGAT |
| | | CTGGGGGCGGGGGGGGGCGGGGGGAATGGGTCTTTTCTAAATT |
| | | GTTAAAAGCAGTTCATGCCATTATTCT |
| | | TAATAAACATTTCTAATATGCTGTGAAA |
| RBM3 Exon | 1 | tggt cgttgtcaag gaccgggaga ctcagcggtc caggggtttt ggtttcatca |
| 3 (Human) | | ccttcaccaa cccagagcat gcttcagttg ccatgagagc catgaacgga gag |
| nt 207-313 of | | |
| NM_006743.5 | | |
| RBM3 Exon | 2 | tctctgg atggtcgtca gatccgtgtg gatcatgcag gcaagtctgc tcggggaacc |
| 4 (Human) | | agaggaggtg gctttggggc ccatgggcgt ggtcgcagct actctagag |
| nt 314-419 of | | |
| NM_006743.5 | | |
| RBM3 | 59 | GCTCCTCTCGATTCCCGGAAGCGGCGAGGCAAATTGCTGCCTCC |
| mature | | GCCTTCCTCGTCCTCTTCCCAACCCC |
| mRNA | | CCCCCCCCGCTGGCCTCAACCAATGAGCACGCACGCCAGGGCG |
| (Mouse) | | CGCTGGGGGACGTTGTGTGACGTCTCG |
| NM_001166409.2 | | GCTACGCACGCTTTATCAGGCGTCTTCCCGCGCCGCAGTCTCTC |
| | | TGTTCTCCCGGTTCCTTCGAGCTCGT |
| | | CGTCTCTGCCGTCCTCTGACTTTTAATTTCCAGGACTTGCCTTCT |
| | | GCCATGTCGTCTGAAGAAGGGAAAC |
| | | TCTTCGTAGGAGGGCTCAACTTCAACACCGATGAACAGGCACTT |
| | | GAAGACCACTTCAGCAGCTTTGGGCC |
| | | TATCTCTGAGGTGGTTGTTGTCAAGGACCGGGAGACTCAAAGAT |
| | | CCCGGGGTTTTGGCTTCATCACCTTC |
| | | ACAAACCCAGAGCATGCCTCAGATGCGATGAGAGCTATGAATGG |
| | | AGAGTCCCTGGATGGGCGCCAAATCC |
| | | GAGTTGATCATGCAGGAAAGTCTGCCAGGGGATCCAGAGGGGG |
| | | TGCCTTTGGTGGGCGTGGTCGCAGTTA |
| | | CTCTAGAGGTGGTGGAGACCAGGGATATGGAAGTGGAAGATATG |
| | | ACAGTCGTCCTGGAGGATATGGATAT |
| | | GGGTATGGGCGGTCTAGAGACTACAGTGGCAGAAGCCAGGGTG |
| | | GCTATGACCGCTACTCAGGAGGAAATT |
| | | ACAGAGACAATTATGACAACTGAGATGGGGCATGCACACAAAAT |
| | | GTAGATACACAAGGAATAACACTTCT |
| | | GATCCAGGATCGTCCTTGCAAATTGCTGTATTTATAAAGATTTTTG |
| | | GAGCTGCGCTGAAACGTCTGTTTT |
| | | AGTACATCAAGTTCTATTTTTGAATTGAGCTCCCAAGGTAGTTTGT |
| | | TAAAGAACTTTTAGAAAGCTCCAT |
| | | GTGTTCTTTAAACATTTTTTCCCTTTTAAAAACAAATTTTAAGACAT |
| | | TTGTTACAGTCCCAATATTTTTC |
| | | CTTTTATCAGAATTTTTGTTTAGGCACTCAGGATTATTGTTTCTGG |
| | | CCAAAAGAAAAAAGTGGCCAGGCT |
| | | AAATCAGTTTTTAAAGTTGATGTGTATTTAGGGACATTTTAAAACC |
| | | TTGTAAACAATGTTTATCATGGCC |
| | | AGAAAGTGATTTCTGAATGTACCTATGAACAATCCGAGTCAAGAT |
| | | CATGATTGCTTAGAGATGTTTTTTA |
| | | TTAAAGATCACCATCCTGATTATATAGAAGAGCTTCTTTAAAATGT |
| | | TTGTGAATGTCATTTTTTAATACT |
| | | GGAAGAAAAGTATTCTGTTGTGTCTGATGTGTTTAGGATGTCCTT |
| | | TATGGAGCTAATTAAAAGATGAAAC |
| | | CTGAACACAAATCAATAC |
| RBM3 Exon | 3 | tggttgttg tcaaggaccg ggagactcaa agatcccggg gttttggctt catcaccttc |
| 3 (Mouse) | | acaaacccag agcatgcctc agatgcgatg agagctatga atggagag |
| nt 362-468 of | | |
| NM_001166409.2 | | |
| RBM3 Exon | 4 | tc cctggatggg cgccaaatcc gagttgatca tgcaggaaag tctgccaggg |
| 4 (Mouse) | | gatccagagg gggtgccttt ggtgggcgtg gtcgcagtta ctctagag |
| nt 469-568 of | | |
| NM_001166409.2 | | |

Aspects and embodiments of the present invention will now be discussed with reference to the accompanying figures. Further aspects and embodiments will be apparent to those skilled in the art. All documents mentioned in this text are incorporated herein by reference.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### SUMMARY OF THE FIGURES

Embodiments and experiments illustrating the principles of the invention will now be discussed with reference to the accompanying figures in which:

FIGS. 1A to 1F. Rbm3 intron 3 contains an evolutionary conserved heat-induced poison exon. FIG. 1A. Sashimi blot identifying a novel exon (E3a; with 7 premature termination codons: PTC) within Rbm3 intron 3. Mouse primary hepatocytes were incubated at 34° C. or 38° C. with or without the translation inhibitor cycloheximide (CHX, DMSO as solvent control) and analysed by RNA sequencing. Below the simplified exon-intron structure, the Sashimi plot shows the distribution of raw sequencing reads (in the y axis). Exon-Exon junction reads are indicated by the numbers connecting the exons. Note that exon 3a is mainly included at 38° C. in CHX. At the bottom, high sequence conservation across placental species is indicated. FIGS. 1B and 1C. Rbm3 exon 3a regulation is conserved in humans. HEK293 cells were pre-entrained with square-wave temperature cycles (12h 34° C./12h 38° C.) for 48h (1B). For the last 24h, cells were treated with DMSO or CHX every 4 h and harvested after 4 h and analyzed by splicing sensitive RT-PCR. In 1C, cells were incubated at the indicated temperatures for 12h (DMSO/CHX last 4h) and analyzed by splicing sensitive RT-PCR. Statistical significance was determined by unpaired t-test and is indicated by asterisks: p values: *p<0.05, **p<0.01, ***p<0.001 (n=3, mean±SD). FIG. 1D. Gene expression of Rbm3 anti-correlates with inclusion of exon 3a. Transcripts per million (TpM) values for Rbm3 are derived from RNA sequencing data from HEK293 cells incubated for the indicated time points for 12 hours and are plotted on the right y-axis (light grey, n=2, mean±SD). Inclusion levels for exon 3a are derived from 1C. FIG. 1E. Sequence alignment of SEQ ID NO: 5 and SEQ ID NO: 6. Mouse RBM3 exon 3a is located at GRCm39 reference genome chromosome X 8010684 to 8010414 (SEQ ID NO: 5). Human RBM3 exon 3a is at GRCh38 reference genome chromosome X 48575815 to 48576083 (SEQ ID NO: 6). Both sequences are shown in Table 1. FIG. 1F. Rbm3 exon 3a stabilization in response to NMD factor knockdown and rescue experiments. Sequencing data were obtained from SRP083135¹⁷.

FIGS. 2A to 2H. Rbm3 exon 3a controls temperature dependent RBM3 expression.

FIG. 2A. CRISPR/CAS9 mediated removal of Rbm3 exon 3a. One of two guide RNAs targeting the upstream intron (#1, #2) were co-transfected with a guide RNA targeting the downstream intron (#3). Below, genotyping PCR after clonal selection of HEK293. px459 transfected cells after clonal selection serve as a negative control. FIGS. 2B and 2C. RT-qPCR (in 2B) and Western blot (in 2C) analysis of Rbm3 levels in edited cell lines. Clonal cell lines from FIG. 2A were incubated at 35° C. or 40° C. for 24h. Isolated RNA was investigated by qPCR and Rbm3 expression is shown relative to GAPDH levels (n=2-3). In 2C, lysates from an independent experiment were investigated for Rbm3 protein expression, hnRNP L served as a loading control. FIG. 2D. Western blot analysis of Rbm3 levels in edited cell lines incubated at 37° C. or 39° C. for 24h. Lysates were investigated for Rbm3 protein expression, hnRNP L served as a loading control. FIG. 2E. Manipulation of Rbm3 exon 3a splicing with morpholinos directly controls Rbm3 expression levels. HEK293 cells were transfected for 48 h at 37° C. with morpholinos blocking either the 3′ss or the 5′ss of exon 3a. Rbm3 expression is shown relative to GAPDH levels and normalized to a non-targeting CTRL morpholino. Statistical significance was determined by unpaired t-test and is indicated by asterisks: p values: **p<0.01 (n>4). FIGS. 2F and 2G. Blocking Rbm3 exon 3a inclusion via blocking morpholinos induces Rbm3 protein levels. HEK293 cells (in 2F) or N2A cells (in 2G) were transfected with the indicated morpholinos for 48 hours. HEK293 cells were shifted to 39° C. for the last 24h. N2A cells were incubated at the indicated temperatures (35° C., 37° C. or 39° C.) for the last 24h. Rbm3 protein levels were investigated by Western blotting. hnRNP L or GAPDH served as a loading control. FIG. 2H. Inhibition of CLK1/4 kinase by TG003 abolishes the effect of temperature on Rbm3 expression. Whippet derived TpM values are shown relative to DMSO 35° C. This reveals an almost 2-fold difference in Rbm3 levels comparing 6h DMSO 35° C. vs 39° C. Note that this is basically abolished by adding TG003 during the shift from 39° C. to 35° C. Data from Haltenhof et al, 2020.

FIGS. 3A to 3K: Minigene mutagenesis allows mapping of cis-regulatory elements controlling Rbm3 exon 3a inclusion.

FIG. 3A. The minigene structure containing the whole unshorten sequence from E3 to E4 (including upstream 3′ss and downstream 5′ss) is shown. FIGS. 3B, 3C. An Rbm3 minigene reproduces temperature-controlled exon 3a inclusion. Minigenes from mouse only or mouse and human were transfected into HEK293 (3B) or mouse N2A cells (3C) and incubated at the indicated temperatures for >12h. Exon 3a inclusion was investigated by splicing sensitive PCR. In 3B, a representative gel image is shown (top). The asterisk marks a product derived from an alternative 3′ss at nucleotide 107 in Exon 3a. Quantification of NMD isoform formation in HEK293 is shown (bottom) (n=3; unpaired t-test derived p-value **p<0.01, ***p<0.001). In 3C, quantification of NMD isoform formation in N2A is shown (n=3; unpaired t-test derived p-value **p<0.01, ***p<0.001). FIGS. 3D and 3E. Systematic mutational screening for regulatory elements in mouse minigene. The indicated sequences (3D) were replaced by sequences from human beta-globin (M3 contains an internal alternative 3′ss at nucleotide 107 of exon 3a (shown with an asterisk and line), M6 contains a 5′ss and the remaining mutants contain exon 2 sequences). Mutations resulting in exon skipping in heat are highlighted in grey (M2 and M4). Analysis of NMD exon inclusion in mutants M1 to M6 at 33° C. and 39° C., in HEK293 and N2A cells (3E). FIGS. 3F and 3G. Detailed mutational screening of the M2 region. In M2 del. the M2 sequence is removed (and not replaced) (3F). In M2-1 to M2-9 the indicated sequences are replaced by human beta-globin sequences. A representative PCR image after transfection of each mutant of FIG. 3F into HEK293 at 39° C. (3G). Mutations resulting in exon skipping (M2, M2 deletion, M2-2, M2-3, M2-4, M2-6, M2-7 and M2-9) are highlighted in grey. FIG. 3H. Detailed mutational screening of the M2 region (borders indicated on the left) and M4 region (borders indicated on the right). In M2 or M4 del. the M2 or M4 sequences are removed (and not replaced). In M2-1 to M2-9 or M4-1 to M4-7 the indicated sequences are replaced by human beta-globin exon 2 sequences from the same relative position. On top a representative PCR image is shown. Below, quantifications of the detected isoforms (n=2). FIGS. 3I and 3J. Temperature response of the indicated minigenes. In 3I, we used the replacement of the M2 and M4 sequences. In 3J, we deleted the evolutionary conserved core of the M2 and M4 regions. Briefly, M2-2 and M2-3 are 100% conserved between the human and mouse. Thus, M2-2 and M2-3 are regarded as core sequence of the M2 enhancer that was then deleted in the hRBM3 minigene. For the M4 region, M4-3 is the central region of the conserved sequence. Therefore, M4-5 and a part of the upstream sequence of M4-4 were deleted as core sequence of the M4 mutant in hRBM3 minigene. FIG. 3K. Systematic mutational screening for regulatory elements. Analysis of fold change in % E3a in HEK293 at 37° C. and 39° C. (mean±SD, n=2).

FIGS. 4A to 4J. Screening for oligonucleotides that control Rbm3 expression.

FIG. 4A. Oligonucleotides targeting either the enhancer element M2-9 (lower left), the enhancer element M4-7 (lower right) or the 5′ss were designed. M2-9 corresponds to exon 3a nucleotides 42-71, and M4-7 corresponds to nucleotides 152-171. FIGS. 4B-4F. Efficient Rbm3 induction in N2A cells. ASOs were transfected for ˜24h, Rbm3 induction was measured relative to a CTRL ASO (see Table 6) and relative to HPRT expression. FIG. 4B. Effect of oligonucleotides targeting the M2-9 element on Rbm3 expression at 39° C. FIG. 4C. Effect of oligonucleotides targeting the M4-7 element on Rbm3 expression at 39° C. FIG. 4D. Effect of oligonucleotides targeting the 5′ss element on Rbm3 expression at 39° C. FIG. 4E. Effect of the combination of two oligonucleotides (MOE and M2D, MOE and M2A, and M2D and M2A) on Rbm3 expression (same CTRL samples) at 39° C. FIG. 4F. Effect of MOE and M2D on Rbm3 expression at 37° C., including an additional control with non-transfected cells (N2A_37° C.). FIG. 4G. Effect of MOE+2 on Rbm3 expression at 37° C., including an additional control with non-transfected cells (N2A_37° C.). FIG. 4H. A morpholino oligonucleotide corresponding to the M2Db sequence induces RBM3 expression in a dose dependent manner (concentrations: 0.5, 1 and 3 μM); both at 37° C. and at 39° C. (highlighted in grey). Morpholinos were transfected for 48h. FIG. 4I. The PS and MOE modified M2 Da efficiently induces Rbm3 expression in human HEK293 cells. M2 Da induced RBM3 expression 1.5-fold. FIG. 4J. M2D induces Rbm3 mRNA expression. ASOs were transfected for 24 hours at 37° C. or at 40° C. in human Hela cells. Rbm3 induction was measured relative to a CTRL ASO at 37° C. and relative to GAPDH expression (mean±SD, n=3, all individual data points are shown; unpaired t-test derived p-value ****p<0.0001).

FIGS. 5A and 5D. In vivo administration of ASOs targeting exon 3A induce RBM3 expression and reduce exon 3A inclusion in brain without cooling.

FIG. 5A. Western blot showing levels of RBM3 after treatment with M2D, M2Db, MOE+2 and M4D, a scrambled control ASO (SCRM) and PBS only. n=2 mice per condition. Mice received 100 μg of scrambled or RBM3 ASOs via intracerebroventricular injection. Hippocampal RBM3 levels were analysed by western blot 3 weeks post-injection. FIG. 5B. Quantification of RBM3 levels relative to PBS-injected control mice from western blot of FIG. 5C. 2 mice per group. FIG. 5C. ASOs (300 μg) induce RBM3 protein expression in vivo. Hippocampus samples from two independent mice per condition were analyzed by Western blotting (top) and RBM3 protein was quantified relative to actin and PBS (bottom, n=2). FIG. 5D. ASOs targeting M2, M4 or 5′ss reduce E3a inclusion in vivo (300 μg dose per mouse). Cerebellum RNA samples from two independent mice per condition were analyzed by splicing sensitive RT-PCR and % E3a signal was quantified (n=2).

FIGS. 6A to 6D. Rbm3 exon 3a controls temperature dependent RBM3 expression (additional data). CRISPR/CAS9 mediated removal of RBM3 E3a was performed as described in relation to FIG. 2A and in Example 2. FIG. 6A. An RT-PCR in CHX treated Hek293 cells confirms CRISPR/CAS9-mediated removal of E3a in an ΔE3a clone at the RNA level. FIGS. 6B, 6C and 6D. RT-qPCR (6B) and Western blot (6C and 6D) analysis of Rbm3 levels in edited cell lines. Clonal cell lines from 6A were incubated at the indicated temperatures for 24h. In 6B, isolated RNA was investigated by qPCR and Rbm3 expression is shown relative to Gapdh levels. In 6C, a representative western blot showing RBM3 protein as detected. In 6D, lysates from an independent experiment were investigated for RBM3 protein expression, hnRNP L served as a loading control (mean±SD, n=6 (3 per clone, indicated in black/grey), all individual data points are shown). Statistical significance was determined by unpaired t-tests and is indicated by asterisks *p<0.05, ***p<0.001, ****p<0.0001.

FIGS. 7A to 7D. Screening for oligonucleotides that control human Rbm3 expression (human ASOs shown in Table 7)

FIG. 7A. ASOs targeting M2-9, M4-7 or the 5′ss (see FIG. 7B) prevent Rbm3 E3a inclusion in human Hela cells. ASO-transfected cells were kept for 24 hours at 40° C. Control samples at 37° C. and 40° C. are shown; CHX was added for the last 4 hours. Exon 3a inclusion was investigated by splicing sensitive RT-PCR, a representative gel and phosphorimager quantification are shown (mean±SD, n=3). The asterisk marks the use of internal 5′ and 3′ss that is promoted by all ASOs targeting the M4 region. ASOs targeting the 5′ss induced the usage of an internal 5′ss (marked by two asterisks). FIG. 7B. ASOs targeting M2-9, M4-7 or the 5′ss prevent Rbm3 E3a inclusion in human Hela cells. ASO-transfected cells were kept for 24 hours at 40° C. Control samples at 37° C. and 40° C. are shown; CHX was added for the last 4 hours. Exon 3a inclusion was investigated by splicing sensitive RT-PCR. A quantification is shown (n=2; for all M2D variants and M2E n=5, for M4B n=1). The asterisk marks the use of internal 5′ and 3′ss that is promoted by all ASOs targeting the M4 region. Note that all variants targeting the M2D region almost quantitatively prevent E3a inclusion, to a level that is lower than the one observed for control cells at 37° C. FIGS. 7C and 7D show representative gels that were used to quantify the inclusion of Exon 3a in FIG. 7B.

FIGS. 8A to 8E. M2D elevates hippocampal RBM3 levels and is profoundly neuroprotective in vivo

FIG. 8A. Schematic of experimental design. Prion-inoculated tg37+/− mice were injected with 200 μg of either M2D or a scrambled ASO at 3 w.p.i. FIG. 8B. Western blot of hippocampal lysates from prion-infected mice treated with scrambled ASO or M2D. M2D increases RBM3 expression by 2-fold compared to scrambled-treated mice, 9 weeks after ASO injection, at 12 w.p.i. FIG. 8C. Representative images of haematoxylin and eosin stained brain slices from normal brain homogenate (NBH) or prion-treated mice, that were additionally treated with scrambled or M2D-ASO. Slices were prepared at 12 w.p.i. when scrambled-treated mice were culled for prion signs. M2D confers marked neuroprotection in the hippocampus, with conservation of CA1-3 pyramidal layer, protection from shrinkage of the whole hippocampus, as well as reduced spongiform change. FIG. 8D. NeuN counts of pyramidal neurons in NBH versus scrambled-treated and M2D-treated prion mice. M2D confers neuroprotection near to levels seen in NBH mice. FIG. 8E. Semi-quantitative scoring of spongiosis in NBH, scrambled-treated and M2D-treated prion mice. Sections showing no signs of spongiosis were scored 0, severe spongiosis was scored 4 (White et al., 2008). M2D significantly reduced spongiosis compared to scrambled-treated prion mice.

### EXAMPLES

In the following Examples, the inventors demonstrate that a previously unannotated Rbm3 exon, referred to herein as exon 3a, is responsible for heat-induced splicing-coupled nonsense-mediated decay (NMD) of RBM3. The inventors further map specific regions that can be targeted to control expression of RBM3. An increase in RBM3 expression was achieved by antisense oligonucleotides targeting these regions.

### Example 1: Rbm3 Intron 3 Contains an Evolutionary Conserved Heat-Induced Poison Exon

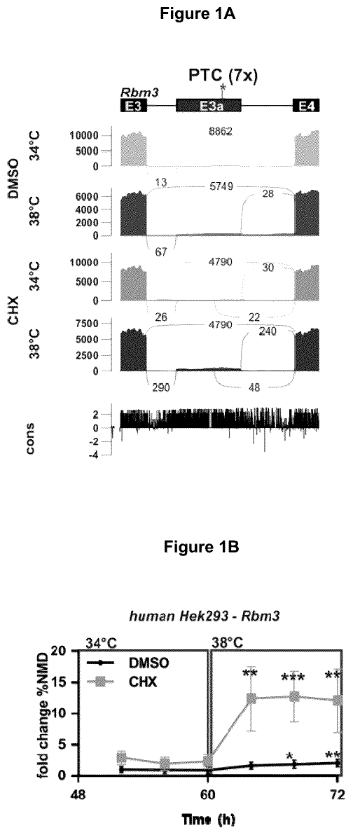

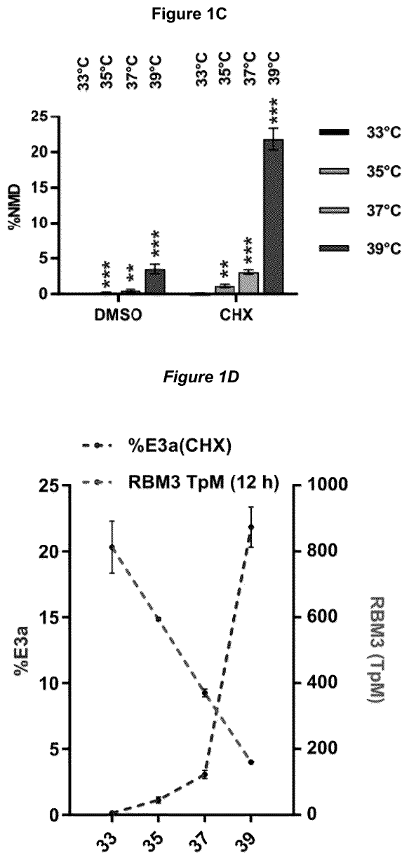

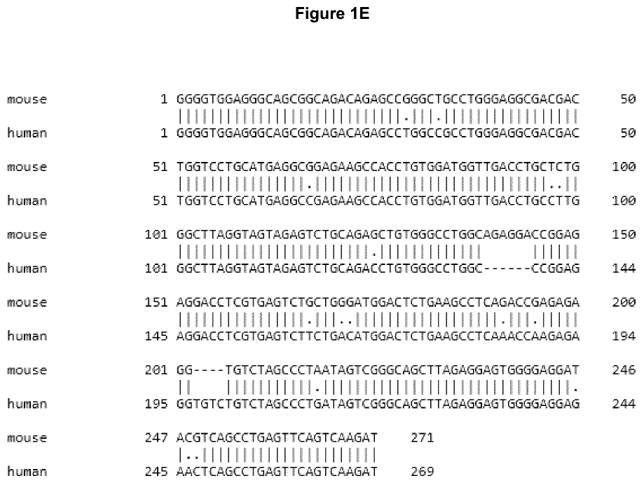

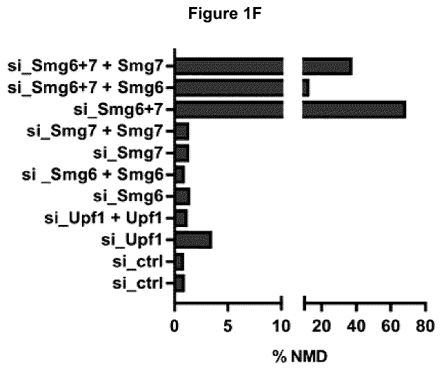

Surprisingly, a more focused analysis of mouse primary hepatocytes RNA sequencing data revealed an unannotated exon (exon 3a, E3a) within the evolutionary conserved intron 3 that escaped our initial global analysis (FIG. 1A). This exon is absent at cold temperatures (34° C.), detectable at warm temperatures (38° C.) and becomes strongly stabilized upon NMD inhibition via cycloheximide (CHX) as demonstrated by the higher number of exon 3-exon 3a and exon 3a-exon 4 junction reads in CHX (290 and 240 at 38° C. in CHX, versus 67 and 28 at 38° C. in DMSO) and the higher read density corresponding to exon 3a in CHX versus DMSO at 38° C. (FIG. 1A). Exon 3a contains seven premature termination codons (PTCs) (FIG. 1A, top).

Consistent with high evolutionary conservation (FIG. 1A, bottom) and an almost identical exon 3a sequence in human, temperature-controlled RBM3 exon 3a inclusion was found in human HEK293 cells (FIGS. 1B, 1C and 1E). RBM3 exon 3a responds quickly (within 4 hours of exposure to a higher temperature of 38° C.) to an external square wave temperature rhythm (FIG. 1B) and could therefore control RBM3 expression in response to circadian body temperature rhythms^{9,16}. Additionally, exon 3a inclusion is temperature responsive within the physiologically relevant temperature range of 33° C. to 39° C. (FIGS. 1C and 1D) correlating with highly temperature-sensitive RBM3 expression. RBM3 exon 3a inclusion is especially prominent at mild heat shock temperature (39° C.) and could therefore be involved in reduced RBM3 expression upon febrile temperature.

The presence of seven PTCs within exon 3a (FIG. 1A) and the strong stabilization upon addition of the translation inhibitor CHX (NMD is a translation-coupled process) suggest that the exon 3a isoform undergoes NMD-mediated degradation. To provide further evidence for degradation via the NMD pathway, we investigated exon 3a stabilization in response to NMD factor knockdowns¹⁷. UPF1 binds to a PCT-containing mRNA and promotes the recruitment of NMD factors such as SMG6 and SMG7 and their assembly into an NMD-activating complex.

Consistent with NMD-mediated degradation, the RBM3 exon 3a isoform is stabilized upon UPF1, SMG6 or SMG7 knockdown and dramatically stabilized (˜75% NMD isoform) upon SMG6/7 double knockdown (FIG. 1F). Exon 3a stabilization could be reversed in rescue experiments (si_Smg6+7+Smg7, si_Smg6+7+Smg6). The higher reversal in the Smg6 rescue sample indicates more prominent SMG6- than SMG7-dependent NMD degradation of the exon 3a isoform.

In summary, these data identify an unannotated poison exon in RBM3, exon 3a, as being responsible for heat-induced splicing-coupled NMD because exon 3a inclusion is observed at higher temperatures and leads to NMD-mediated degradation. The cold-induced expression of RBM3 may therefore be attributed to the heat-dependent inclusion of the poison exon 3a.

### Example 2: Rbm3 Exon 3a Controls Temperature-Dependent RBM3 Expression

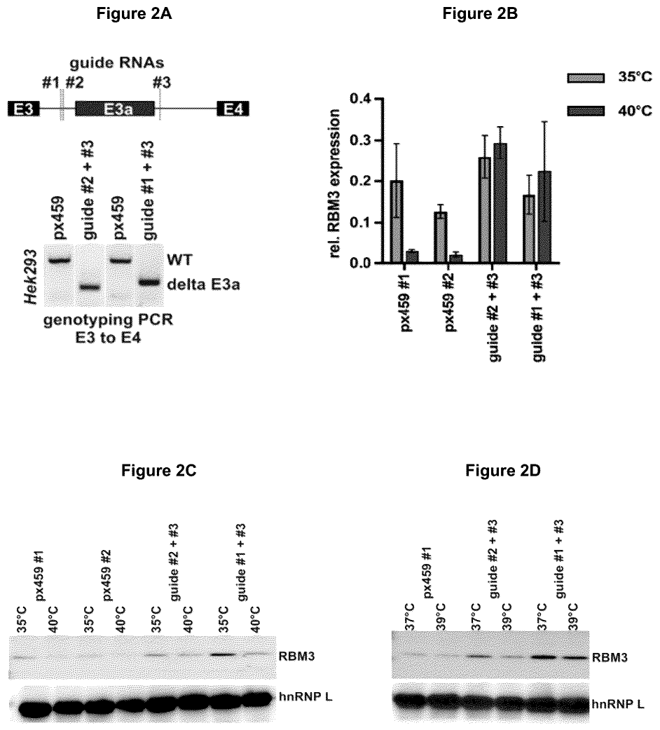

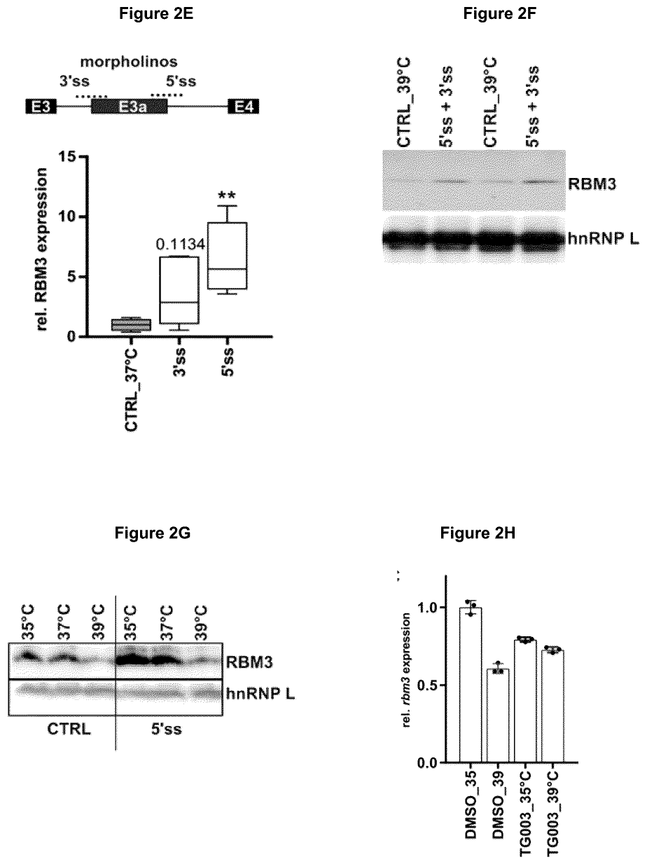

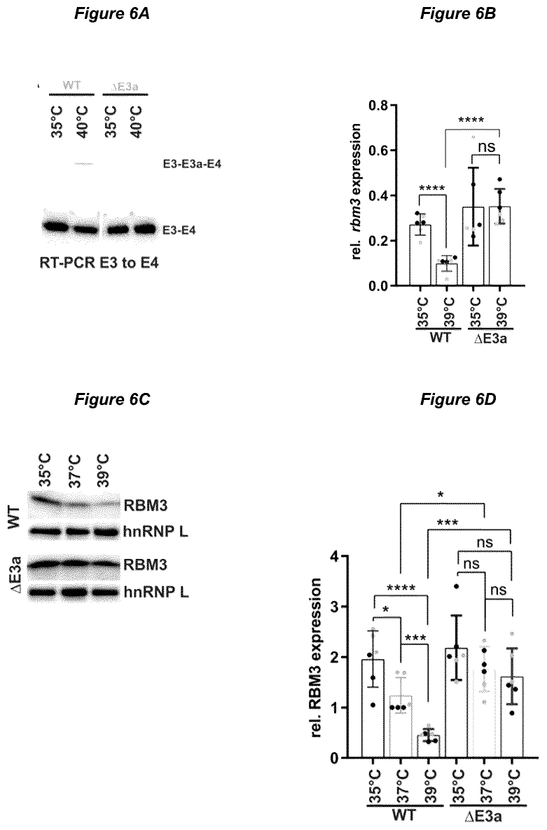

To investigate whether RBM3 exon 3a is involved in temperature-controlled RBM3 expression CRISPR/Cas9-mediated genome editing was used to generate cell lines lacking RBM3 exon 3a. After clonal selection two homozygous cell lines, derived from distinct guide RNA pairs sgRNA #2 and sg RNA #3, and sgRNA #1 and sgRNA #3 (FIG. 2A) were obtained. While control cell lines-generated from px459 empty vector transfected cells-showed strong temperature-controlled RBM3 mRNA and protein expression, this was almost abrogated in cell lines lacking exon 3a (FIGS. 2B and 2C, and FIGS. 6B, 6C and 6D). Consistent with stronger exon 3a inclusion in heat (see Example 1), the increase in RBM3 protein expression in the cell lines lacking exon 3a over the control cell lines is more prominent in heat (FIGS. 2C and 2D); but also even at 37° C., removal of exon 3a induces RBM3 expression (FIG. 2D).

Importantly, cell lines lacking E3a did not only lose Rbm3 temperature sensitivity but showed a constantly high Rbm3 expression level, that in control cells was only reached at low temperature. These data suggest that temperature-controlled alternative splicing, coupled to NMD is the main mechanism that controls Rbm3 expression levels in the physiologically relevant temperature range. Temperature-dependent phosphorylation of SR proteins likely contributes to this regulation (Preussner et. al., Body Temperature Cycles Control Rhythmic Alternative Splicing in Mammals. Mol. Cell 67, 433-446.e434 (2017). https://doi.org: 10.1016/j.molcel.2017.06.006; Haltenhof et al., A Conserved Kinase-Based Body-Temperature Sensor Globally Controls Alternative Splicing and Gene Expression. Mol. Cell, 78, 57-69.e4 (2020)), as the effect of temperature on Rbm3 expression is strongly reduced in conditions with inhibited CDC-like kinases (CLKs; Haltenhof et al., 2020 and FIG. 2H).

Next, splice-site blocking morpholinos were used to block RBM3 exon 3a inclusion and directly measure the effect on gene expression levels. Morpholinos targeting either the 3′ splice site or the 5′ splice of exon 3a induced Rbm3 mRNA levels in HEK293, with the 5′ splice site blocking morpholino having a more robust effect (FIG. 2E) as it induced mRNA levels up to ten-fold. Morpholinos also induced RBM3 protein expression at 39° C. (FIG. 2F) and at 37° C. (FIG. 2G), confirming that Rbm3 expression can be controlled in trans by targeting splicing of E3a.

Together, these data provide strong evidence for splicing-controlled RBM3 expression and identify exon 3a as a target for therapeutic increase of RBM3 expression, without cooling.

### Example 3: Minigene Mutagenesis Allows Mapping of Cis-Regulatory Elements Controlling Rbm3 Exon 3a Inclusion

One way of manipulating (non-productive) alternative splicing is the use of antisense oligonucleotides. Antisense oligonucleotides have broad therapeutic potential^{18,19}, and have been used to induce exon skipping^{20,21}. Therefore, the design of the most potent antisense oligonucleotides often requires intensive screening. To narrow down potential target sites for antisense-based therapeutics, we started with a minigene analysis. In a minigene context, NMD isoforms are not degraded because minigenes are not translated. Minigenes allow systematic mutagenesis analysis to decipher cis-regulatory elements.

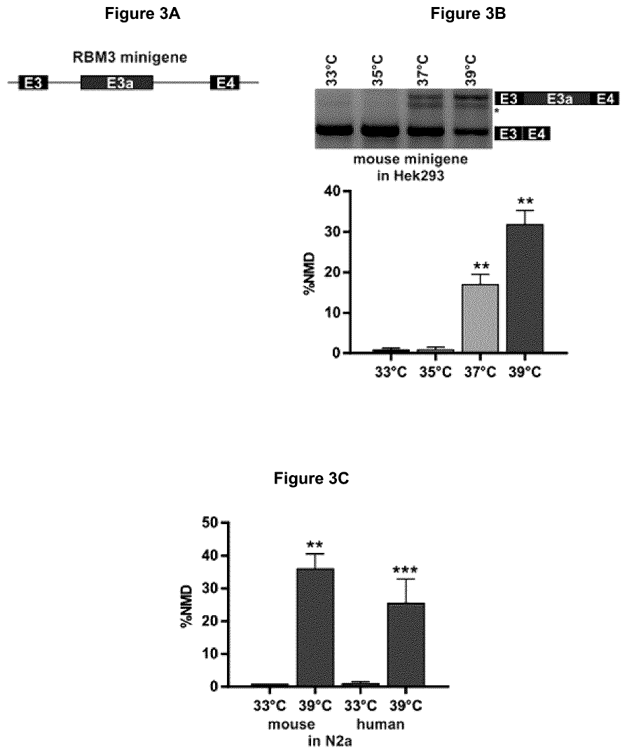

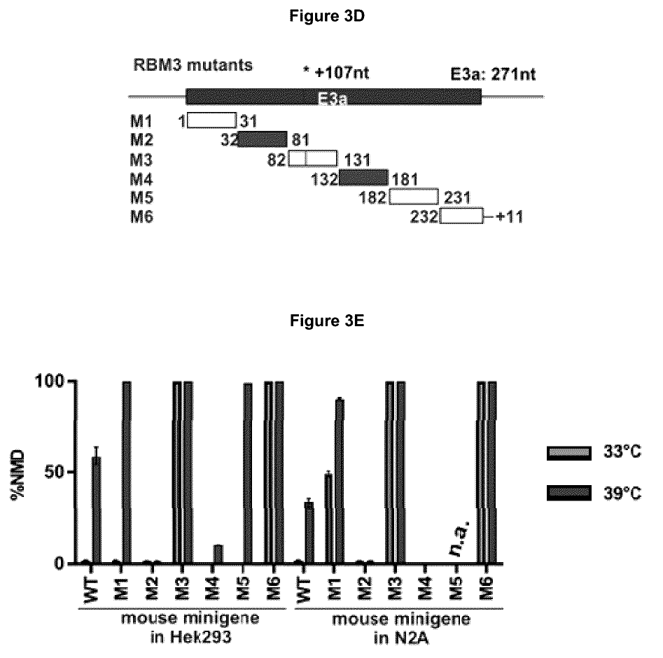

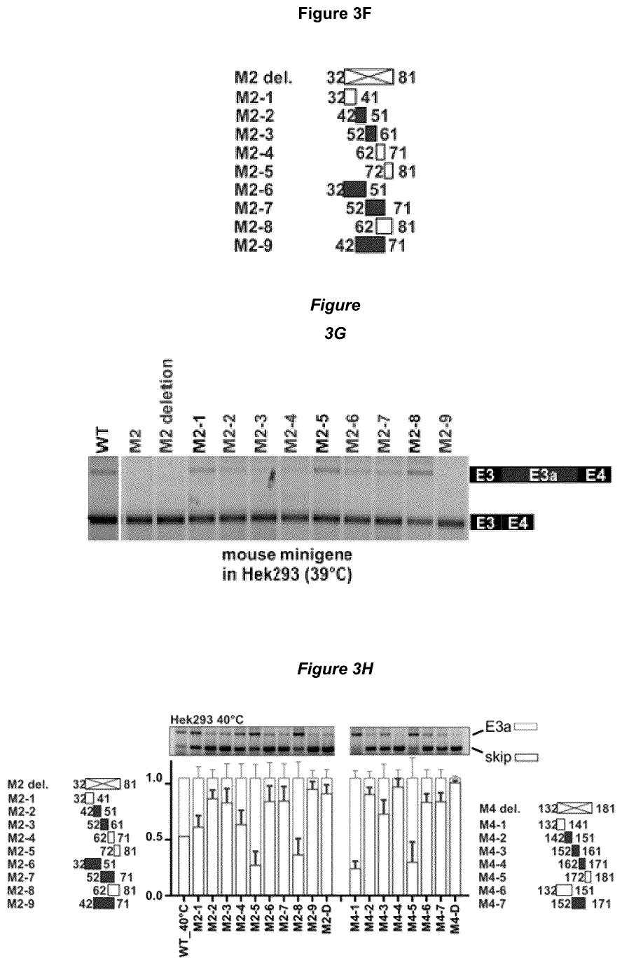

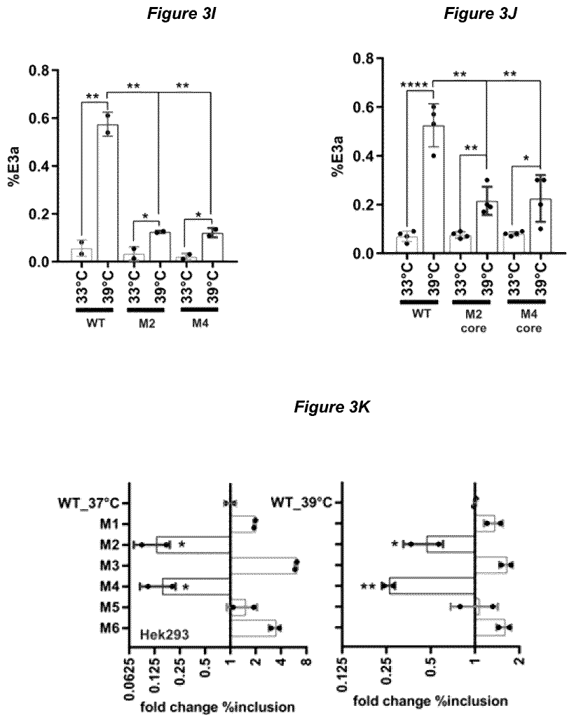

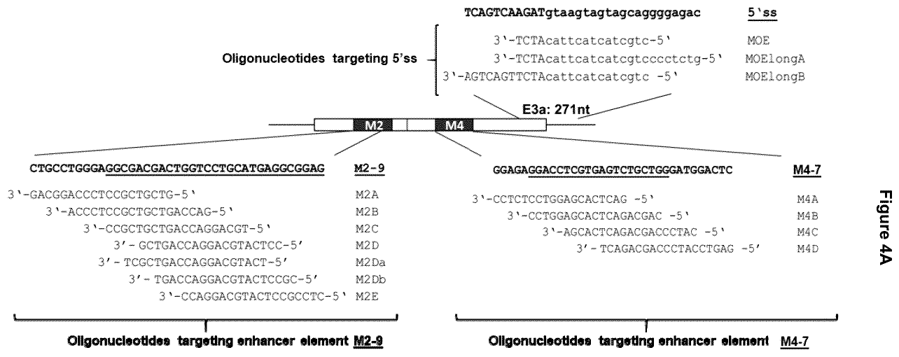

Human or mouse genomic sequences comprising RBM3 exons 3 to 4 (FIG. 3A) were cloned into a minigene and minigene splicing after transfection into human HEK293 or mouse neuronal N2A cells was analysed (FIGS. 3B and 3C). The exon 3a-containing NMD isoform of the minigene is detected at warm temperatures (FIG. 3B), and similar to endogenous splicing, minigene splicing responds to temperature in a gradual manner (FIG. 3B, bottom) and is indistinguishable between species (FIG. 3C), indicating that the minigene contains all cis-regulatory elements required for temperature-controlled alternative splicing. An alternative 3′ss which is internal to exon 3a and is located at nucleotide 107 of exon 3a (for reference, see FIG. 1E) results in temperature-dependent incorporation of part of exon 3a, namely nucleotides 108 to 271, as indicated by the asterisk in FIG. 3B.

Screening mutagenesis replacing 50 nucleotide windows with human beta-globin sequences revealed two strong enhancer elements at windows M2 and M4 (FIGS. 3D and 3E). We also identified silencer elements in the regions downstream of the 3′ss (M1), close to an internal 3′ss (M3) and upstream of the 5′ss (M6) (FIGS. 3D, 3E and 3K). Interestingly, only after mutation of sequences including an internal alternative 3′ splice site (m3) or the 5′ splice site (m6), exon 3a inclusion is enabled at cold temperature (M3, M6 in FIG. 3E). These sequences therefore contain strong silencer elements preventing exon 3a inclusion in cold. In contrast, mutations m2 and m4 prevent exon 3a inclusion, even upon heat, thus encoding enhancer elements (M2, M4 in FIG. 3E). Importantly, deleting the m2 sequence also abolished exon 3a inclusion, and smaller replacements (M2-1 to M2-9) revealed the location of the enhancer elements within nucleotides 42 to 71 (corresponding to M2-2, M2-3, M2-4 and M2-9 in FIGS. 3F and 3G) as these replacements result in exon skipping in heat. Deleting (instead of replacing) the M2 or M4 enhancer sequences also abolished exon 3a inclusion and smaller replacements revealed the location of the enhancer elements within nucleotides 142 to 171 in the M4 region and specifically nucleotides 152-171 (referred to herein as M4-7) as a second similarly strong enhancer element (FIG. 3H).

Additionally, mutating the enhancers M2 or M4 (or deleting their evolutionary conserved core) strongly reduces the splicing response to temperature (FIGS. 31 and 3J).

The “M2 core” is located at nucleotides 42 to 61 of exon 3a and corresponds to sequence ggcgacgactggtcctgcat (SEQ ID NO: 88).

The “M4 core” is located at nucleotides 146 to 160 of exon 3a and corresponds to sequence ggacctcgtgagtct (SEQ ID NO: 89).

The identified enhancer elements at nucleotides 42-71 (M2-9 region) and 152-171 (M4-7 region) of exon 3a could be targeted to prevent exon 3a inclusion.

### Example 4: Screening for Oligonucleotides that Control Rbm3 Expression

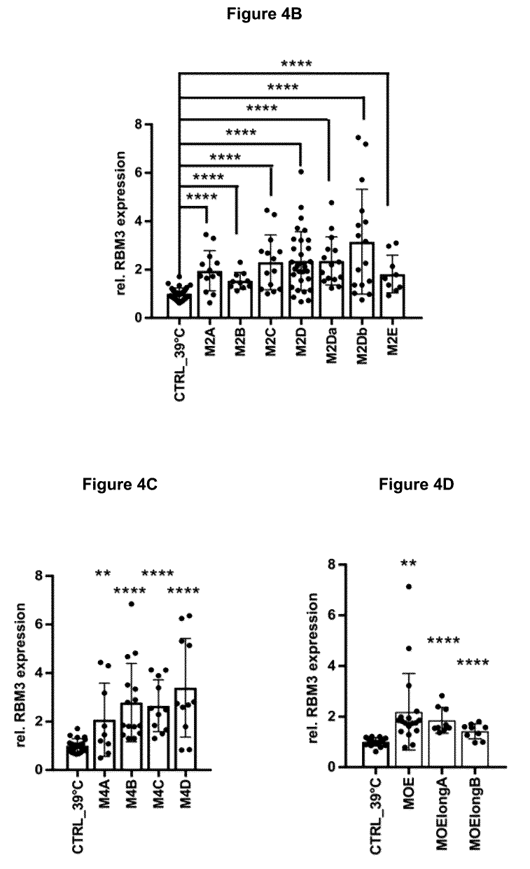

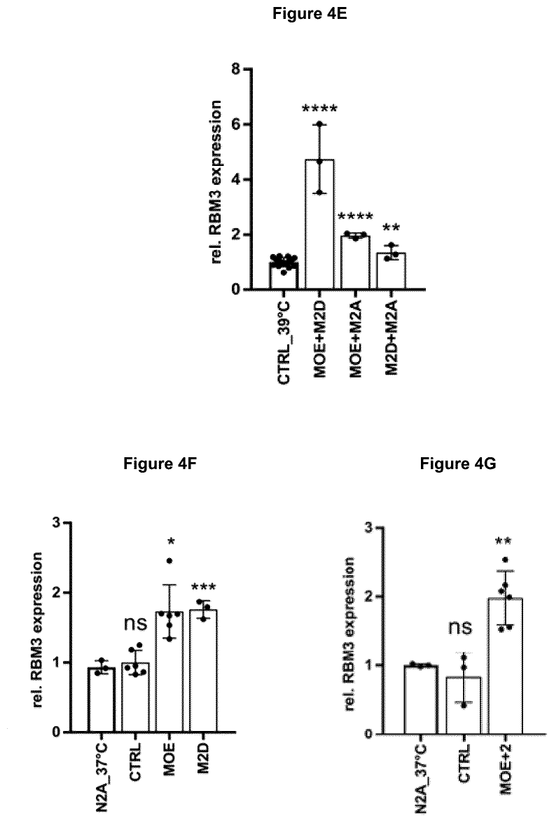

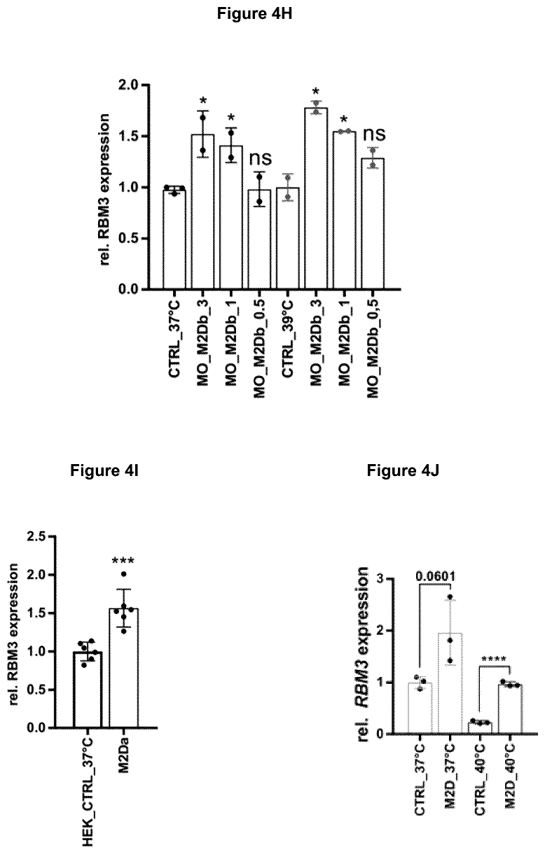

Based on the minigene analysis which identified nucleotides 42-71 (M2-9) in M2 and 152-171 (M4-7) in M4 as regions comprising an enhancer element (Example 3) antisense oligonucleotides (ASOs) against these regions were developed and screened. In addition, based on the morpholino experiments which show that blocking the 5′ splice site of exon 3a induces Rbm3 protein levels (see Example 2 and FIGS. 2E to 2G) antisense oligonucleotides against the 5′ splice site of exon 3a were screened. FIG. 4A shows the sequences of the seven oligonucleotides targeting M2-9, of the four oligonucleotides targeting M4-7 and of the three oligonucleotides targeting the 5′ss of exon 3a.

The ASOs that were tested combined a phosphorothioate (PS) modified backbone with uniform 2′-O-methoxyethyl (MOE) modified bases as in the FDA approved drug nusinersen²², which should allow distribution throughout the central nervous system after intrathecal injection²³. PS/MOE chemistry, as well as morpholino chemistry (as used in Example 2) may also be used for systemic delivery in vivo²⁴.

N2A cells were transfected with each PS/MOE-modified ASO for ˜24 h and Rbm3 induction was measured relative to a control PS/MOE-modified ASO (CTRL) and relative to HPRT expression. The sequences of the antisense oligonucleotides are listed in Table 6.

TABLE 6
Sequences of ASOs used in Example 4. The listed ASOs were PS and MOE modified.
M2Db was also obtained as a morpholino (MO_M2Db; FIG. 4H). The reverse complement
sequence is also provided to show the respective binding site in the RBM3 pre-mRNA.

| Target | | | Reverse complement sequence |
|---|---|---|---|
| in exon 3a | ASO | ASO Sequence 5′ to 3′ | for each ASO |
| 5′ ss | MOE | ctgctactacttacATCT | AGATgtaagtagtagcag |
| | | (SEQ ID NO: 34) | (SEQ ID NO: 93) |
| | MOElong | gtctcccctgctactacttacATCT | AGATgtaagtagtagcaggggagac |
| | A | (SEQ ID NO: 36) | (SEQ ID NO: 94) |
| | MOElong | ctgctactacttacATCTTGACTGA | TCAGTCAAGATgtaagtagtagcag |
| | B | (SEQ ID NO: 38) | (SEQ ID NO: 95) |
| | MOE + 2 | ccctgctactacttacATC | GATgtaagtagtagcaggg |
| | | (SEQ ID NO: 69) | (SEQ ID NO: 96) |
| M2-9 | M2A | GTCGTCGCCTCCCAGGCAG | CTGCCTGGGAGGCGACGAC |
| (nt 42 to 71) | | (SEQ ID NO: 40) | (SEQ ID NO: 97) |
| | M2B | GACCAGTCGTCGCCTCCCA | TGGGAGGCGACGACTGGTC |
| | | (SEQ ID NO: 42) | (SEQ ID NO: 98) |
| | M2C | TGCAGGACCAGTCGTCGCC | GGCGACGACTGGTCCTGCA |
| | | (SEQ ID NO: 43) | (SEQ ID NO: 99) |
| | M2D | CCTCATGCAGGACCAGTCG | CGACTGGTCCTGCATGAGG |
| | | (SEQ ID NO: 44) | (SEQ ID NO: 100) |
| | M2Da | TCATGCAGGACCAGTCGTC | GACGACTGGTCCTGCATGA |
| | | (SEQ ID NO: 46) | (SEQ ID NO: 101) |
| | M2Db | CGCCTCATGCAGGACCAGT | ACTGGTCCTGCATGAGGCG |
| | | (SEQ ID NO: 47) | (SEQ ID NO: 102) |
| | M2E | CTCCGCCTCATGCAGGACC | GGTCCTGCATGAGGCGGAG |
| | | (SEQ ID NO: 49) | (SEQ ID NO: 103) |
| M4-7 | M4A | GACTCACGAGGTCCTCTCC | GGAGAGGACCTCGTGAGTC |
| (nt 152-171) | | (SEQ ID NO: 51) | (SEQ ID NO: 104) |
| | M4B | CAGCAGACTCACGAGGTCC | GGACCTCGTGAGTCTGCTG |
| | | (SEQ ID NO: 52) | (SEQ ID NO: 105) |
| | M4C | CATCCCAGCAGACTCACGA | TCGTGAGTCTGCTGGGATG |
| | | (SEQ ID NO: 54) | (SEQ ID NO: 106) |
| | M4D | GAGTCCATCCCAGCAGACT | AGTCTGCTGGGATGGACTC |
| | | (SEQ ID NO: 56) | (SEQ ID NO: 107) |

| | | |
|---|---|---|
| N/A | CTRL | TTCTCCGAACGTGTCACG |
| | | (SEQ ID NO: 108) |

All seven tested ASOs targeting the enhancer element in the M2 region and all four tested ASOs against the M4 region increased RBM3 levels at 39° C. in N2A cells, with an at least 2-fold increase by the M2D and M4D ASO variants (FIGS. 4B and 4C, respectively).

The ASO targeting the 5′ splice site also increased RBM3 levels up to 2-fold (MOE, FIG. 4D). The effect is not improved by extended ASOs MOElongA and MOElongB.

Combining a M2-targeting (M2D) and a 5′ss-targeting (MOE) ASO further increased RBM3 levels by >4-fold (FIG. 4E). Combination of a different M2-targeting ASO (M2A) with MOE led to a ˜2-fold increase of RBM3 levels, and so did combination of M2A and M2D.

The effect of selected ASOs was tested at lower temperatures (at 37° C.). Both M2D and MOE increase RBM3 levels ˜2-fold even at 37° C. (FIG. 4F). M2D led to higher increase of RBM3 levels at 39° C. which indicates that the enhancer element targeted by M2D is more active at higher temperatures. Importantly, the M2D ASO induces Rbm3 expression at 40° C. to a level observed in control cells at 37° C., and is also inducing Rbm3 expression 2 fold at 37° C. (FIG. 4J).

Mouse N2A cells were also treated with an ASO carrying a 2-nucleotide shifted variant of MOE, which is termed MOE+2 (see Table 6). Like M2D and MOE, MOE+2 induces RBM3 expression 2-fold at 37° C. (FIG. 4G).

In summary, this experiment demonstrates that ASOs targeting exon 3a M2, M4 or 5′ss increase the mRNA and protein levels of RBM3 in N2A cells and identifies ASOs as promising for in vivo application. ASOs M2D, M2Db, M4D and MOE+2 were selected for in vivo studies as described in Example 7.

Mouse N2A cells were treated with an ASO carrying a different modification (morpholino; MO_M2Db) corresponding to the sequence of M2Db (shown in Table 4 and also in Table 6). This ASO induces RBM3 expression in a dose dependent manner at 37° C. and at 39° C. and achieves over 1.5-fold increase in RBM3 expression at 3 μM (FIG. 4H).

HEK293 cells were also transfected with PS and MOE modified M2 Da. M2 Da led to 1.5-fold increase in RBM3 expression at 37° C. (FIG. 4I).

These experiments in human HEK293, together with the effect of M2D in human Hela cells (FIG. 4J), show that morpholino and/or PS and MOE modified ASOs targeting exon 3a M2 are also able to significantly induce expression of RBM3 in human cells.

### Example 5: Screening for Human ASO Sequences that Control Exon 3a Inclusion in Human RBM3

ASOs targeting M2-9, M4-7 or the 5′ss of the human rbm3 exon 3a were designed based on the minigene analysis and are shown in Table 7.

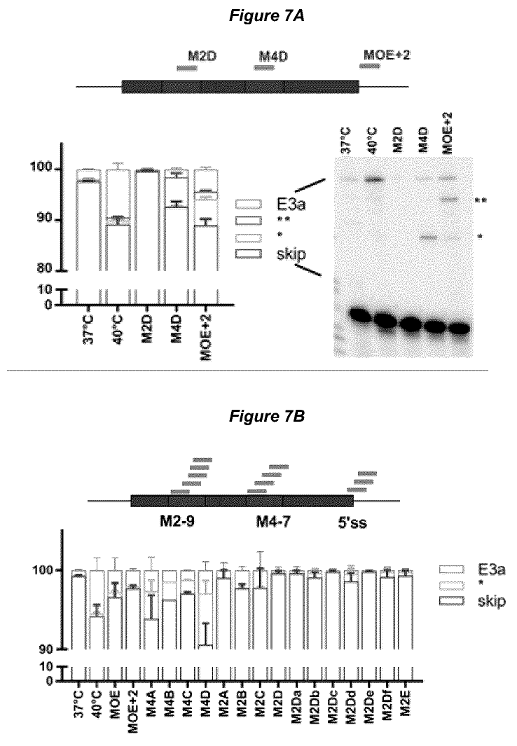

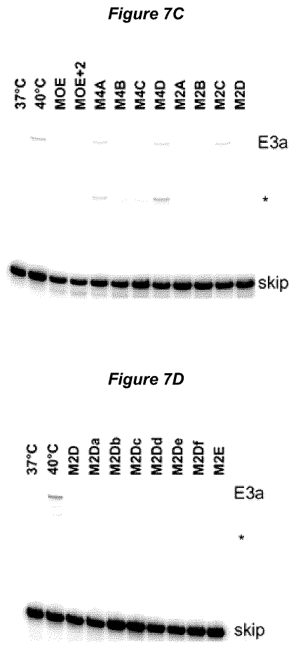

An ASO screen was performed and several ASOs targeting the respective enhancers or the 5′ss in human rbm3 that prevent E3a inclusion in Hela cells were identified (FIGS. 7A and 7B to 7D, and Table 7). While ASOs targeting the M4 region or the 5′ss result in partial usage of alternative 3′ or 5′ splice-sites, all variants targeting the M2D region quantitatively abolish E3a inclusion (FIG. 7D). 5

This data suggests that ASOs targeting human RBM3 exon 3a, and specifically the M2-9 region are promising agents for inhibiting exon 3a inclusion and increasing RBM3 expression in human cells and have a potential therapeutic use.

TABLE 7
Sequences of human ASOs used in Example 5 and FIGS. 7A to 7D. The
listed ASOs were PS and MOE modified. The reverse complement
sequence is also provided to show the
respective binding site in the hRBM3 pre-mRNA.

| Target in | | | Reverse complement sequence |
|---|---|---|---|
| human exon 3a | ASO | ASO Sequence 5′ to 3′ | for each ASO |
| 5′ ss | hMOE | ttgctactacttacatct | agatgtaagtagtagcaa |
| | | (SEQ ID NO: 35) | (SEQ ID NO: 110) |
| | hMOE + 2 | ccttgctactacttacatc | gatgtaagtagtagcaagg |
| | | (SEQ ID NO: 70) | (SEQ ID NO: 111) |
| M2-9 | hM2A | GTCGTCGCCTCCCAGGCGG | CCGCCTGGGAGGCGACGAC |
| | | (SEQ ID NO: 41) | (SEQ ID NO: 112) |
| | hM2B | GACCAGTCGTCGCCTCCCA | TGGGAGGCGACGACTGGTC |
| | | (SEQ ID NO: 42) | (SEQ ID NO: 98) |
| | hM2C | TGCAGGACCAGTCGTCGCC | GGCGACGACTGGTCCTGCA |
| | | (SEQ ID NO: 43) | (SEQ ID NO: 99) |
| | hM2D | CCTCATGCAGGACCAGTCG | CGACTGGTCCTGCATGAGG |
| | | (SEQ ID NO: 44) | (SEQ ID NO: 100) |
| | hM2Da | TCATGCAGGACCAGTCGTC | GACGACTGGTCCTGCATGA |
| | | (SEQ ID NO: 46) | (SEQ ID NO: 101) |
| | hM2Db | GGCCTCATGCAGGACCAGT | ACTGGTCCTGCATGAGGCC |
| | | (SEQ ID NO: 48) | (SEQ ID NO: 113) |
| | hM2Dc | CTCATGCAGGACCAGTCGT | ACGACTGGTCCTGCATGAG |
| | | (SEQ ID NO: 83) | (SEQ ID NO: 114) |
| | hM2Dd | GCCTCATGCAGGACCAGTC | GACTGGTCCTGCATGAGGC |
| | | (SEQ ID NO: 84) | (SEQ ID NO: 115) |
| | hM2De | ATGCAGGACCAGTCGTCGC | GCGACGACTGGTCCTGCAT |
| | | (SEQ ID NO: 85) | (SEQ ID NO: 116) |
| | hM2Df | TCGGCCTCATGCAGGACCA | TGGTCCTGCATGAGGCCGA |
| | | (SEQ ID NO: 87) | (SEQ ID NO: 117) |
| | hM2E | CTCGGCCTCATGCAGGACC | GGTCCTGCATGAGGCCGAG |
| | | (SEQ ID NO: 50) | (SEQ ID NO: 118) |
| M4-7 | hM4A | GACTCACGAGGTCCTCTCC | GGAGAGGACCTCGTGAGTC |
| | | (SEQ ID NO: 51) | (SEQ ID NO: 104) |
| | hM4B | CAGAAGACTCACGAGGTCC | GGACCTCGTGAGTCTTCTG |
| | | (SEQ ID NO: 53) | (SEQ ID NO: 119) |
| | hM4C | CATGTCAGAAGACTCACGA | TCGTGAGTCTTCTGACATG |
| | | (SEQ ID NO: 55) | (SEQ ID NO: 120) |
| | hM4D | GAGTCCATGTCAGAAGACT | AGTCTTCTGACATGGACTC |
| | | (SEQ ID NO: 57) | (SEQ ID NO: 121) |
| N/A | CTRL | TTCTCCGAACGTGTCACG | CGTGACACGTTCGGAGAA |
| | | (SEQ ID NO: 108) | (SEQ ID NO: 109) |

### Example 6: Materials and Methods Used in Examples 1-4

### RNA-Seq Analysis and Bioinformatics

RNA sequencing data from mouse primary hepatocytes are deposited under GSE158882⁵. Sequencing data from Hela cells after knockdown and rescue of Upf1, Smg6 and Smg7 were obtained from SRP083135¹⁷. Mapping of reads to reference genomes (mm10 for mouse, hg38 for human) was performed using STAR version 2.5.3a²⁵. The RBM3 Sashimi plots was generated using a customized version of ggsashimi²⁶, which additionally displays conservation scores. Percent spliced in (PSI) values for RBM3 exon 3a after knockdown and depletion was manually calculated from junction read counts.

### Tissue Culture Cells

HEK293T and N2A cell stocks are maintained in liquid nitrogen and early passage aliquots are thawed periodically. To rule out mycoplasma contamination, cell morphology is routinely assessed and monthly checked using a PCR-based assay. HEK293T cells were cultured in DMEM high glucose with 10% FBS and 1% Penicillin/Streptomycin. N2A cells were cultured in 50% OptiMEM/50% OptiMEM Glutamax with 10% FBS and 1% Penicillin/Streptomycin. Mouse hippocampal neurons were isolated and maintained as previously described (Peretti et al. Life Sci Alliance. 2021; 4(4):e202000884. doi: 10.26508/lsa.202000884). All cell lines were usually maintained at 37° C. and 5% CO₂.

For temperature experiments, cells were shifted into pre-equilibrated incubators for the indicated times. For square-wave temperature cycles we used two incubators set to 34° C. and 38° C. and shifted the cells every 12 hours²⁷.

Transfections of HEK293T or N2A cells (for minigenes or CRISPR guides) using Rotifect (Roth) were performed according to the manufacturer's instructions. Cycloheximide (Sigma) was used at 40 μg/ml final concentration or DMSO as solvent control.

For morpholino experiments, cells were seeded and transfected 1 day later using Endoporter following to the manufacturer's manual.

Morpholinos (Rbm3 5′ss: GTCTCCCCTGCTACTACTTACATCT (SEQ ID NO: 36); 3′ss: CCTCCACCCCCTAGAACAGAAGGCA (SEQ ID NO: 60); and standard control) and Endoporter transfection reagent were purchased from Gene Tools.

PS/MOE-modified antisense oligonucleotides (1 μl, 100 ng/μl) were purchased from Mycrosynth and transfected into N2A cells or HEK293T cells (3*10⁶ cells per well on a 12-well plate) via Rotifect, and then 4 hours later the transfected N2A cells were transferred into 39 degree (or 37 degree) incubator, followed by RNA extraction 24 hrs later and RT-qPCR.

The morpholino targeting mouse exon 3a M2-9 (MO_M2Db) was transfected into N2A cells at 0.5, 1 and 3 μM for 48 hours at 37° C. and at 39° C.

### RT-PCR and RT-qPCR

RT-PCRs were done as previously described²⁸. Shortly, RNA was extracted using RNATri (Bio&Sell) and 1 μg RNA was used in a gene-specific RT reaction.

Endogenous RBM3 splicing was analyzed with a radioactively labeled forward primer in exon 3 (5′-TCATCACCTTCACCAACCCA (SEQ ID NO: 7)) and a reverse primer in exon 5 (5′-TCTAGAGTAGCTGCGACCAC (SEQ ID NO: 8)).

For analysis of minigene splicing, the RNA was additionally digested with DNase I and re-purified. Minigene splicing was investigated with minigene specific primers:

T7fwd:
(SEQ ID NO: 61)
5′-GACTCACTATAGGGAGACCC;
BGHrev:
(SEQ ID NO: 62)
5′-TAGAAGGCACAGTCGAGG.

For qRT-PCR, the Rbm3 gene-specific primer was combined with a housekeeping gene reverse primer in one RT reaction. qPCR was then performed in a 96-well format using the Blue S′Green qPCR Kit Separate ROX (Biozym) on Stratagene Mx3000P instruments. qPCRs were performed in technical duplicates, mean values were used to normalize expression to a housekeeping gene (human: GAPDH; mouse: HPRT); DCT, and D(DCT)s were calculated for different conditions.

TABLE 7
Primers used in qPCR

| Primer name | Primer sequence | SEQ ID NO | Organism |
|---|---|---|---|
| mRBM3_E2/3_qF | AAGGGAAACTCTTCGTAG | 122 | qPCR mouse |
| mRBM3_E3_qR | GACAACAACCACCTCAGAGATAG | 123 | qPCR mouse |
| HPRT_qF | CAACGGGGGACATAAAAGTTATTGGTGGA | 124 | qPCR mouse |
| HPRT_qR | TGCAACCTTAACCATTTTGGGGCTGT | 125 | qPCR mouse |
| hRBM3_E2/3_qF | AAGGAAAGCTCTTCGTGGGA | 126 | qPCR human |
| hRBM3_E3_qR | GACAACGACCACCTCAGAGA | 127 | qPCR human |
| GAPDH_qF | CTTCGCTCTCTGCTCCTCCTGTTCG | 128 | qPCR human |
| GAPDH_qR | ACCAGGCGCCCAATACGACCAAAT | 129 | qPCR human |

### Generation of CRISPR-Cas9 Edited Cells and Analysis of Rbm3 Expression

For genome-engineering in HEK293 cells, sequences flanking exon 3a of Rbm3 were analyzed for sgRNA candidates in silico using the Benchling tool. A pair of oligonucleotides for the highest ranked candidate sgRNA²⁹ upstream and downstream of the exon was synthesized and subcloned into the PX459 vector.

sgRNA sequence #1:
(SEQ ID NO: 11)
5′-TGTGTCTGCTCGGGGCAGCG;
sgRNA sequence #2:
(SEQ ID NO: 12)
5′-CCTGTGAGTGGGCACTGCG;
sgRNA sequence #3:
(SEQ ID NO: 13)
5′-TCCTGATGAAGCCATTCTG.

Cells were co-transfected with guide RNA #3 and either #1 or #2 in 6-well plates using Rotifect (Roth) following the manufacturer's instruction. 48 hours after transfection, the transfected cells were selected with 1 μg/ml puromycin and clonal cell lines were isolated by dilution²⁹. Genomic DNA was extracted using DNA extraction buffer (200 mM Tris/HCl PH 8.3, 500 mM KCl, 5 mM MgCl2, 0.1% gelatin in H₂O) and to confirm the exon knockout on DNA level, a genotyping PCR was performed using primers binding in introns upstream and downstream of the cutting sites (FWD: 5′-ATCTGCAGAGGGACCTTGTC (SEQ ID NO:63); REV: 5′-CAGACTTGCCTGCATGATCC (SEQ ID NO: 64)). In promising clones the exon knockout was additionally confirmed after RNA isolation by splicing sensitive PCR using one forward primer in exon 3 and one reverse primer in exon 4 (data not shown). Rbm3 total expression levels were investigated by RT-qPCR and Western blot.

### Western Blot

Whole-cell extracts (WCEs) were prepared with lysis buffer (20 mM Tris (pH 8.0), 2% NP-40 (v/v), 0.01% sodium deoxycholate (w/v), 4 mM EDTA and 200 mM NaCl) supplemented with protease inhibitor mix (Aprotinin, Leupeptin, Vanadat and PMSF). Concentrations were determined using RotiNanoquant (Roth), according to manufacturer's instructions. SDS-PAGE and Western blotting followed standard procedures. Western blots were quantified using the ImageQuant TL software. The following antibodies were used for Western blotting: hnRNPL (4D11, Santa Cruz), Rbm3 (14363-1-AP, proteintech), Gapdh (GT239, GeneTex).

### Minigene Constructs

Cloning was performed using PCR introducing HindIII and XhoI sites, and ligation into pcDNA3.1 (+). Constructs were cloned using a forward primer in the intron upstream of exon 3 (mouse: 5′-AATTTAAGCTTctgtggctgtgcctggct (SEQ ID NO: 65); human: 5′-AAC TTA AGC TTT CCG GCC ACC CTT TGC TAC (SEQ ID NO: 66)), a reverse primer in the intron downstream of exon 4 (mouse: 5′-AATTTCTCGAGttcagacataggctcttaacatt (SEQ ID NO: 67); human: 5′-TAG ACT CGA GAT AGG CAA CTC TCC CTC TCA C (SEQ ID NO: 68)) and human or mouse DNA as template.

For each of mRbm3 mutant and sub-mutant cloning, the mutated sequences were deleted or replaced by sequences from human beta-globin (M3 contains a 3′ss, M6 a 5′ss and the remaining mutants exon 2 sequences). Briefly, two DNA fragments were amplified from the mRbm3 minigene with PCR primer pairs introducing the mutation. Then, these two DNA fragments were used as templates for a PCR to get the full-length mRbm3 mutants. All minigene sequences were confirmed by sequencing (Microsynth SeqLab).

### Example 7: ASOs Targeting Exon 3A Induce RBM3 Expression in Mice

### 7.1 Results

Tolerability studies involved injection of 4 different RBM3 targeting ASOs (M2D, M2Db, MOE+2 and M4D) and a scrambled control (SCRM) as well as a PBS only control. n=2 for each ASO. Doses of 30 μg, 100 μg and 300 μg per mouse were all well tolerated for the 3 weeks before sampling with no ill effects associated with ASO administration¹ (unsteady gait, weight loss, seizure etc.).

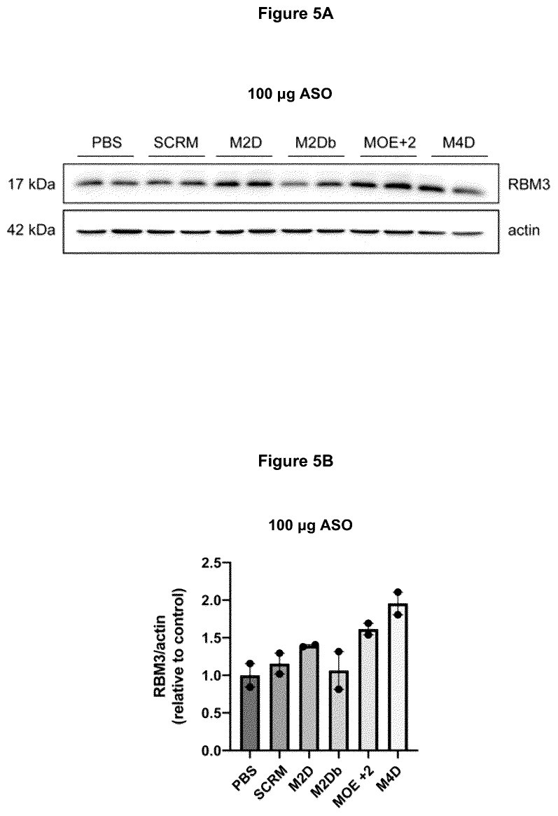

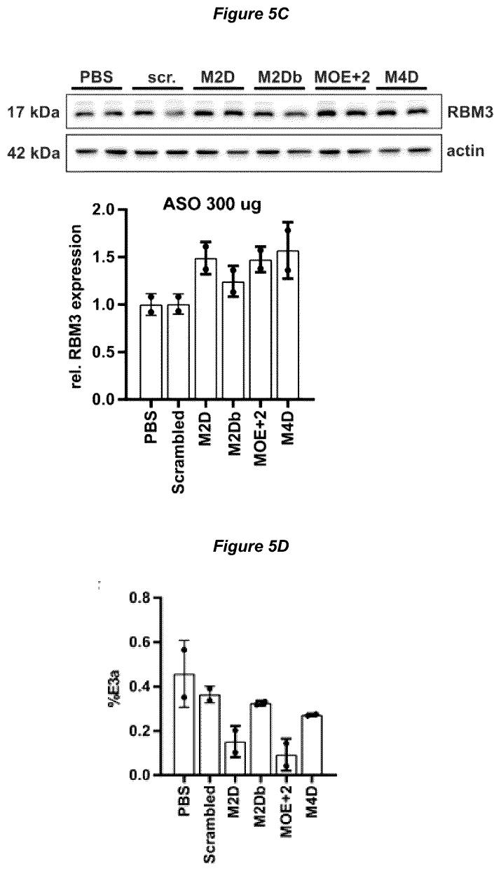

Efficacy: All ASOs increased RBM3 expression in the hippocampus by up to 1.5 fold at each dose as determined by western blot (FIGS. 5A, 5B and 5C), correlating with reduced E3a inclusion levels (FIG. 5D).

This upregulation seen by the ASOs is similar to RBM3 levels upon cooling, shown to be neuroprotective in multiple models²⁻⁴. The lack of effect seen with M2Db may be due to lack of efficacy due to the stability or pharmacokinetics of this ASO, or may reflect low numbers tested.

In summary, RBM3 targeting ASOs (M2D, MOE+2 and M4D) that are complementary to different regions with respect to RBM3 exon 3A induce RBM3 expression in mice without cooling to similar levels that occur on hypothermia.

### REFERENCES

1. A. L. Southwell et al., In vivo evaluation of candidate allele-specific mutant huntingtin gene silencing antisense oligonucleotides. Molecular therapy: the journal of the American Society of Gene Therapy 22, 2093-2106 (2014).
2. A. Bastide et al., RTN3 Is a Novel Cold-Induced Protein and Mediates Neuroprotective Effects of RBM3. Curr Biol 27, 638-650 (2017).
3. D. Peretti et al., RBM3 mediates structural plasticity and protective effects of cooling in neurodegeneration. Nature 518, 236-239 (2015).
4. D. Peretti et al., TrkB signaling regulates the cold-shock protein RBM3-mediated neuroprotection. Life science alliance 4, (2021).

### 7.2 Material and Methods

### Administration of ASOs

All ASOs (Merck) were diluted in a final volume of 10 μl in sterile PBS, this was delivered to mice by intracerebroventricular injection to the left lateral ventricle at stated doses (30 μg, 100 μg or 300 μg). Mice were first anaesthetized using isoflurane before mounting on a stereotaxic frame. An incision was made along the midline of the scalp and the skull exposed. A small hole was drilled through the skull at 0.3 mm anterior and 1 mm lateral to Bregma, a Hamilton 33G neuro-syringe was lowered to 3 mm below the surface of the skull and ASOs were injected over 1 minute, the needle was removed slowly 2 minutes after cessation of injection. Head wounds were sealed with glue before surgical recovery. Mice were monitored daily for 3 weeks before sampling. Mice were killed by cervical dislocation and brain was immediately dissected into tubes and flash frozen in liquid nitrogen. Samples take were: hippocampus, pre-frontal cortex and cerebellum.

### Sample Preparation

Hippocampi were lysed on ice in 350 μL of RIPA buffer (50 mM Tris pH 8, 50 mM NaCl, 1% IGEPAL, 0.5% sodium deoxycholate, 0.1% SDS) (Sigma) supplemented with protease and phosphatase inhibitors (Roche). Samples were sonicated at 4° C. then centrifuged at 13,000 rpm for 10 minutes to remove cell debris. Protein concentration was determined using the Bicinchoninic Acid (BCA) assay (Pierce).

### Western Blotting

15 μg of lysate was resolved by SDS-PAGE, transferred onto 0.2 μm nitrocellulose membrane and blocked in 5% bovine serum albumin (BSA). Membranes were incubated overnight with RBM3 antibody (1:2000, Proteintech). Actin was used as a loading control (1:5000, Cell Signaling). Blots were imaged on a BioRad ChemiDoc and quantified using ImageJ.

### Example 8: ASO Treatment Elevates Hippocampal RBM3 Levels and is Profoundly Neuroprotective

The therapeutic potential of ASO-mediated E3a exclusion in prion disease was tested in a model which has been extensively used to test the effects of cooling and RBM3 over-expression on the progression of neurodegeneration (Peretti, 2015; Bastide, 2017. Curr. Biol. 27, 638-650, doi:10.1016/j.cub.2017.01.047; Peretti, 2021), as well as of many other interventions (Mallucci, 2003. Science (New York, N.Y.) 302, 871-874, doi:10.1126/science. 1090187).

Hemizygous tg37+/− mice overexpress prion protein (PrP) at around 3-fold over wild-type levels (Mallucci, 2002 EMBO J. 21, 202-210, doi:10.1093/emboj/21.3.202). When inoculated with Rocky Mountain Laboratory (RML) prions, these mice show a rapid incubation time, succumbing to disease in only 12 weeks post inoculation (w.p.i.), with rapidly progressive extensive neurodegeneration throughout the brain, including hippocampal CA1-3 regions (Mallucci, 2002).

Early cooling (at 3 w.p.i.) to boost RBM3 levels, or lentiviral delivery of RBM3 to the hippocampus, are profoundly neuroprotective in both prion-diseased tg37 mice and Alzheimer's 5×FAD mice, whereas RNAi of RBM3 eliminates the protective effects of cooling (Peretti, 2015).

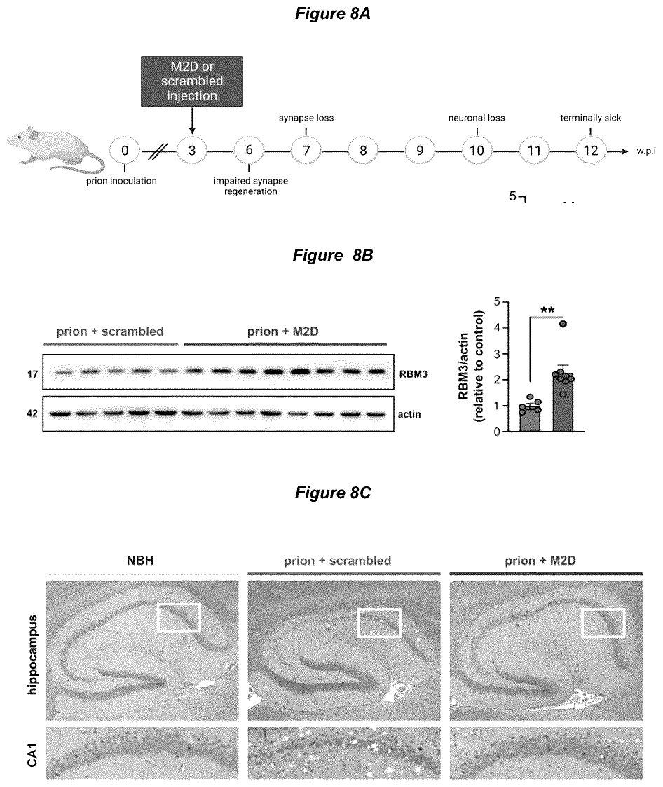

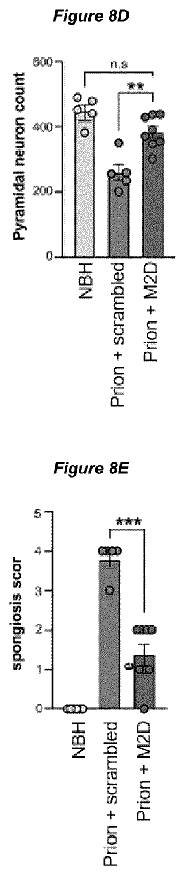

To address whether ASO-mediated induction of RBM3 expression via E3a skipping—in the absence of cooling—is similarly neuroprotective, we treated prion-diseased tg37 mice (n=8) with 200 μg of either M2D or a scrambled ASO control. (FIG. 8A; this dose was chosen as an average of the efficacious doses of 100 and 300 μg).

ASOs were delivered by a single intracerebroventricular injection at 3 w.p.i., consistent with the timing of our previous interventions (early cooling, or LV-RBM3, Peretti, 2005). Mice treated with M2D/scrambled ASO were analysed for neuroprotection at 12 w.p.i. when all scrambled-ASO treated mice had succumbed to prion disease. Remarkably, a single dose of M2D ASO resulted in RBM3 levels 2-fold higher than scrambled-treated mice 9 weeks after injection, at 12 w.p.i. (FIG. 8B). Higher levels of RBM3 were associated, as before (Peretti, 2015) with marked neuroprotection: 7/8 M2D-treated mice showed extensive preservation of pyramidal neurons in the hippocampal CA1-3 regions (FIGS. 8C and 8D) compared to scrambled-ASO treated mice, of which 5 of 6 mice showed profound neuronal loss in these regions (FIGS. 8C and 8D). M2D-mediated neuroprotection was also associated with reduced spongiosis, with M2D-treated mice showing markedly lower spongiosis scores compared to scrambled-treated mice (FIG. 8E).

These data support a robust and long-lasting induction of RBM3 without cooling by a single dose of M2D, 9 weeks previously, with remarkable neuroprotection in the context of a rapidly progressive neurodegenerative disorder. The ability to raise RBM3 levels by one administration of a well-tolerated ASO using FDA-approved chemistry, in place of therapeutic hypothermia, has strong implications for neuroprotection in diverse conditions, from the acute setting treatment of neonates through to cardiac surgery, stroke and head injury in adults, to longer term neuroprotection in degenerative disorders. In the acute setting, this approach brings the neuroprotection of RBM3 expression while bypassing the substantial risks associated with intensive care and cold, such as coagulation problems, pneumonia and invasive monitoring. Further, the approach has marked appeal in the prevention of neurodegeneration. ASOs are highly successful in children with SMA, and have been recently licensed for the treatment of the rapidly progressive adult neurodegenerative disorder, ALS (Miller, T. M. et al. Trial of Antisense Oligonucleotide Tofersen for SOD1 ALS. N Engl J Med 387, 1099-1110, doi:10.1056/NEJMoa2204705 (2022)). In the search for disease-modifying therapies for Alzheimer's disease and related dementias, induction of the neuroprotective effects of RBM3 via ASO delivery to drive its long-term expression is a compelling therapeutic approach, boosting neuronal resilience and synapse regeneration that are key to resisting the direct and indirect toxic effects of these protein misfolding disorders, particularly in the context of aging.

### 8.1 Experimental Details

### Mice

All animal work conformed to UK Home Office regulations and performed under the Animal [Scientific Procedures] Act 1986, Amendment Regulations 2012 and following institutional guidelines for the care and use of animals for research. All studies were ethically reviewed by the University of Cambridge Animal Welfare and Ethical Review Body (AWERB). Mice were housed in groups of 2-5 animals/cage, under 12 hours light/dark cycle and were tested in the light phase. Water and standard mouse chow were given ad-libitum. Mice were randomly assigned treatment groups by cage number. Experimenters were blind to group allocation during the experiments and when assessing clinical signs. For behavioral testing no formal randomization was needed or used. Procedures were fully compliant with Animal Research: Reporting of In Vivo Experiments (ARRIVE) guidelines.

### Prion Infection of Mice

3-week-old tg37+/− mice were inoculated intra-cerebrally into the right parietal lobe with 30 μL of 1% brain homogenate of Chandler/RML (Rocky Mountain Laboratories) prions under general anesthetic, as described (Mallucci, 2002). Animals were culled when they developed clinical signs of scrapie as defined in Mallucci, 2002; Mallucci, 2003; Mallucci, 2007 Neuron 53, 325-335, doi:10.1016/j.neuron.2007.01.005. Control mice received 1% normal brain homogenate.

### REFERENCES

A number of publications are cited above in order to more fully describe and disclose the invention and the state of the art to which the invention pertains. Full citations for some of these references are provided below. The entirety of each of these references is incorporated herein.
1 Danno, S. et al. Increased transcript level of RBM3, a member of the glycine-rich RNA-binding protein family, in human cells in response to cold stress. Biochem. Biophys. Res. Commun. 236, 804-807, doi:10.1006/bbrc. 1997.7059 (1997).
2 Nishiyama, H. et al. A glycine-rich RNA-binding protein mediating cold-inducible suppression of mammalian cell growth. J. Cell Biol. 137, 899-908, doi:10.1083/jcb.137.4.899 (1997).
3 Ciuzan, O., Hancock, J., Pamfil, D., Wilson, I. & Ladomery, M. The evolutionarily conserved multifunctional glycine-rich RNA-binding proteins play key roles in development and stress adaptation. Physiol. Plant. 153, 1-11, doi:10.1111/ppl. 12286 (2015).
4 Jackson, T. C. et al. Cold stress protein RBM3 responds to temperature change in an ultra-sensitive manner in young neurons. Neuroscience 305, 268-278, doi: https://doi.org/10.1016/j.neuroscience.2015.08.012 (2015).
5 Neumann, A. et al. Alternative splicing coupled mRNA decay shapes the temperature-dependent transcriptome. EMBO Rep 21, e51369, doi:10.15252/embr.202051369 (2020).
6 Lykke-Andersen, S. & Jensen, T. H. Nonsense-mediated mRNA decay: an intricate machinery that shapes transcriptomes. Nature Reviews Molecular Cell Biology 16, 665, doi:10.1038/nrm4063 https://www.nature.com/articles/nrm4063 #supplementary-information (2015).
7 Haltenhof, T. et al. A conserved kinase-based body temperature sensor globally controls alternative splicing and gene expression. Mol. Cell (2020).
8 Hoekstra, M. M., Emmenegger, Y., Hubbard, J. & Franken, P. Cold-inducible RNA-binding protein (CIRBP) adjusts clock-gene expression and REM-sleep recovery following sleep deprivation. Elife 8, doi:10.7554/eLife.43400 (2019).
9 Morf, J. et al. Cold-inducible RNA-binding protein modulates circadian gene expression posttranscriptionally. Science (New York, N.Y.) 338, 379-383, doi:10.1126/science. 1217726 (2012).
10 Lujan, D. A., Ochoa, J. L. & Hartley, R. S. Cold-inducible RNA binding protein in cancer and inflammation. Wiley Interdiscip Rev RNA 9, doi:10.1002/wrna. 1462 (2018).
11 Qiang, X. et al. Cold-inducible RNA-binding protein (CIRP) triggers inflammatory responses in hemorrhagic shock and sepsis. Nat. Med. 19, 1489-1495, doi:10.1038/nm.3368 (2013).
12 Chip, S. et al. The RNA-binding protein RBM3 is involved in hypothermia induced neuroprotection. Neurobiology of Disease 43, 388-396, doi: https://doi.org/10.1016/j.nbd.2011.04.010 (2011).
13 Zhu, X. et al. RBM3 promotes neurogenesis in a niche-dependent manner via IMP2-IGF2 signaling pathway after hypoxic-ischemic brain injury. Nature communications 10, 3983, doi:10.1038/s41467-019-11870-x (2019).
14 Yang, H. J. et al. Cold-inducible protein RBM3 mediates hypothermic neuroprotection against neurotoxin rotenone via inhibition on MAPK signalling. J. Cell. Mol. Med. 23, 7010-7020, doi:10.1111/jcmm. 14588 (2019).
15 Peretti, D. et al. RBM3 mediates structural plasticity and protective effects of cooling in neurodegeneration. Nature 518, 236-239, doi:10.1038/nature 14142 (2015).
16 Liu, Y. et al. Cold-induced RNA-binding proteins regulate circadian gene expression by controlling alternative polyadenylation. Sci Rep 3, 2054, doi:10.1038/srep02054 (2013).
17 Colombo, M., Karousis, E. D., Bourquin, J., Bruggmann, R. & Mühlemann, O. Transcriptome-wide identification of NMD-targeted human mRNAs reveals extensive redundancy between SMG6- and SMG7-mediated degradation pathways. RNA (New York, N.Y.) 23, 189-201, doi:10.1261/rna.059055.116 (2017).
18 Lim, K. H. et al. Antisense oligonucleotide modulation of non-productive alternative splicing upregulates gene expression. Nature communications 11, 3501, doi:10.1038/s41467-020-17093-9 (2020).
19 Bennett, C. F. Therapeutic Antisense Oligonucleotides Are Coming of Age. Annu. Rev. Med. 70, 307-321, doi:10.1146/annurev-med-041217-010829 (2019).
20 Erdos, M. R. et al. A targeted antisense therapeutic approach for Hutchinson-Gilford progeria syndrome. Nat. Med. 27, 536-545, doi:10.1038/s41591-021-01274-0 (2021).
21 Puttaraju, M. et al. Systematic screening identifies therapeutic antisense oligonucleotides for Hutchinson-Gilford progeria syndrome. Nat. Med. 27, 526-535, doi:10.1038/s41591-021-01262-4 (2021).
22 Hua, Y. et al. Peripheral SMN restoration is essential for long-term rescue of a severe spinal muscular atrophy mouse model. Nature 478, 123-126, doi:10.1038/nature 10485 (2011).
23 Finkel, R. S. et al. Treatment of infantile-onset spinal muscular atrophy with nusinersen: a phase 2, open-label, dose-escalation study. The Lancet 388, 3017-3026, doi: https://doi.org/10.1016/S0140-6736(16) 31408-8 (2016).
24 Sheng, L., Rigo, F., Bennett, C. F., Krainer, A. R. & Hua, Y. Comparison of the efficacy of MOE and PMO modifications of systemic antisense oligonucleotides in a severe SMA mouse model. Nucleic Acids Res. 48, 2853-2865, doi:10.1093/nar/gkaa126 (2020).
25 Dobin, A. et al. STAR: ultrafast universal RNA-seq aligner. Bioinformatics 29, 15-21, doi:10.1093/bioinformatics/bts635% J Bioinformatics (2012).
26 Garrido-Martin, D., Palumbo, E., Guigo, R. & Breschi, A. ggsashimi: Sashimi plot revised for browser- and annotation-independent splicing visualization. PLoS Comput Biol 14, e1006360, doi:10.1371/journal.pcbi. 1006360 (2018).
27 Preussner, M. et al. Body Temperature Cycles Control Rhythmic Alternative Splicing in Mammals. Mol. Cell 67, 433-446.e434, doi:10.1016/j.molcel.2017.06.006 (2017).
28 Preussner, M. et al. Rhythmic U2af26 alternative splicing controls PERIOD1 stability and the circadian clock in mice. Mol. Cell 54, 651-662, doi:10.1016/j.molcel.2014.04.015 (2014).
29 Ran, F. A. et al. Genome engineering using the CRISPR-Cas9 system. Nat Protoc 8, 2281-2308, doi:10.1038/nprot.2013.143 (2013).
30 Williams, D. R. et al. Seasonally hibernating phenotype assessed through transcript screening. Physiol Genomics 24, 13-22 (2005).
31 Smart, F. et al. Two isoforms of the cold-inducible mRNA-binding protein RBM3 localize to dendrites and promote translation. J. Neurochem. 101, 1367-1379 (2007).
32 Llorian, M. et al. The alternative splicing program of differentiated smooth muscle cells involves concerted non-productive splicing of post-transcriptional regulators. Nucleic Acids Research 44, 8933-8950 (2016).
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method for inhibiting non-sense mediated decay of RBM3-encoding mature mRNA in a cell, the method comprising exposing the cell to an agent, wherein the agent is capable of hybridising to a region of the pre-mRNA of RBM3 such as to alter the splicing of the pre-mRNA such that in the resulting mature mRNA exon 3a is not incorporated.

2. The method of claim 1, wherein the cell is a mammalian cell, optionally wherein the cell is a mouse cell or human cell.

3. The method of claim 1 or 2, wherein the cell is a neuron, astrocyte, oligodendrocyte, microglial cell, ependymal cell or brain stem cell.

4. The method of any one of claims 1 to 3, wherein the method is in vitro or ex vivo.

5. The method of any one of claims 1 to 3, wherein the method is in vivo.

6. A method for treating or preventing a disease affected by RMB3 expression in a subject, or providing neuroprotective treatment to a subject, the method comprising administering to the subject an agent, wherein the agent is capable of hybridising to a region of the pre-mRNA of RBM3 such as to alter splicing of the pre-mRNA such that in the resulting mature mRNA exon 3a is not incorporated.

7. The method of claim 6, wherein the treating or treatment is for:
a neurological disease and/or the disease is neonatal hypoxic ischemic encephalopathy, head injury, or stroke;
a neurodegenerative disease optionally selected from Alzheimer's disease, Parkinson's disease, prion disease, frontotemporal dementia, a tauopathy, amyotrophic lateral sclerosis (ALS), and vascular dementia;
neurological damage, optionally caused during cardiac surgery or induced coma; or
depression or anxiety.

8. The method of any one of claims 1 to 7, wherein said region is selected from: a region within exon 3a, a region spanning a splice site of exon 3a, a region located within 250 nucleotides upstream exon 3a and a region located within 250 nucleotides downstream exon 3a.

9. The method of claim 8, wherein said region comprises a splice enhancer element.

10. The method of claim 9, wherein said region corresponds to SEQ ID NO: 27.

11. The method of claim 10, wherein said region corresponds to SEQ ID NO: 29.

12. The method of claim 9, wherein the region corresponds to SEQ ID NO: 15.

13. The method of claim 12, wherein the region corresponds to a SEQ ID NO selected from the group consisting of: SEQ ID NO: 16, 17, 19, 21, 23, and 25.

14. The method of claim 8, wherein said region spans the 5′ splice site of exon 3a.

15. The method of claim 14, wherein said region comprises nucleotides 257 to 269, 258 to 269, 259 to 269, 260 to 269, 261 to 269, 262 to 269, 263 to 269, 264 to 269, 265 to 269, 266 to 269, or 267 to 269 of SEQ ID NO: 6, and/or nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33.

16. The method of claim 8, wherein said region spans the 3′ splice site of exon 3a.

17. The method of claim 16, wherein said region comprises nucleotides 1 and 2, 1 to 3, 1 to 4, 1 to 5, 1 to 6, 1 to 7, 1 to 8, 1 to 9, or 1 to 10 of SEQ ID NO: 6 and/or nucleotides 136 to 147, 137 to 147, 138 to 147, 139 to 147, 140 to 147, 141 to 147, 142 to 147, 143 to 147, 144 to 147, or 145 to 147 of SEQ ID NO: 32.

18. The method of any one of claims 1 to 17, wherein the agent is an antisense oligonucleotide (ASO).

19. The method of claim 18, wherein the ASO is complementary or essentially complementary to all or part of the region defined in claim 8.

20. The method of claim 19, wherein the agent is an ASO which is 100% complementary to all or part of said region.

21. The method of claim 19 or claim 20, wherein the ASO is 10 to 30 nucleotides long, optionally 25 nucleotides long, optionally 15, 16, 17, 18 or 19 nucleotides long, or optionally 19 nucleotides long.

22. The method of claim 19, wherein the ASO comprises or consists of one of SEQ ID NOs: 40, 41, 42, 43, 44, 45, 46, 47, 48, 49, 50, 83, 84, 85, 86 and 87.

23. The method of claim 19, wherein the ASO comprises or consists of one of SEQ ID NOs: 51, 52, 53, 54, 55, 56 and 57.

24. The method of claim 19, wherein the ASO comprises or consists of one of SEQ ID NOs: 34, 35, 36, 37, 38, 39, 69 and 70.

25. The method of any one of claims 19 to 24, wherein the ASO comprises an LNA, RNA or DNA nucleotide.

26. The method of claim 25, wherein the ASO comprises alternating LNA and RNA nucleotides.

27. The method of claim 25, wherein the ASO comprises alternating LNA and DNA nucleotides.

28. The method of claim 25, wherein the ASO comprises alternating RNA and DNA nucleotides.

29. The method of any one of claims 19 to 28, wherein the ASO comprises a backbone modification, which is optionally a phosphorothioate linkage.

30. The method of any one of claims 19 to 29, wherein the ASO comprises a sugar moiety modification, which is optionally 2′-O-methoxyethyl (MOE) modification.

31. The method of claim 29 or claim 30, wherein the ASO comprises a 2′-O-methoxyethyl (MOE) modification and a phosphorothioate linkage.

32. The method of claim 31, wherein the ASO is fully 2′-O-methoxyethyl and fully phosphorothioate modified.

33. The method of any one of claims 19 to 28, wherein the ASO is or comprises a phosphorodiamidate morpholino oligonucleotide.

34. The method of any one of claims 18 to 24, the method comprising expressing the ASO from a transgene encoding the ASO which is introduced into or administered to said cell or subject.

35. The method of claim 34, wherein the viral vector is a recombinant AAV vector.

36. An antisense oligonucleotide (ASO) as defined in any one of claims 19 to 33.

37. A method for inhibiting non-sense mediated decay of RBM3-encoding mature mRNA in a cell, the method comprising exposing the cell to an agent, wherein the agent is the ASO of claim 36.

38. A method of treating or preventing a disease affected by RMB3 expression in a subject, or providing neuroprotective treatment to a subject, the method comprising administering to the subject an agent, wherein the agent is the ASO of claim 36.

39. The method of claim 38, wherein the treating or treatment is for:
a neurological disease and/or the disease is neonatal hypoxic ischemic encephalopathy, head injury, or stroke;
a neurodegenerative disease optionally selected from Alzheimer's disease, Parkinson's disease, prion disease, frontotemporal dementia, a tauopathy, amyotrophic lateral sclerosis (ALS), and vascular dementia; or
neurological damage, optionally caused during cardiac surgery or induced coma; or
depression or anxiety.

40. An agent for use in a method for inhibiting nonsense-mediated decay (NMD) of RBM3-encoding mature mRNA or for use in a method for treating or preventing a disease affected by RMB3 expression, or for providing a neuroprotective treatment, according to any one of claims 1 to 35 or 37 to 39.

41. Use of an agent in the preparation of a medicament for use in a method for inhibiting nonsense-mediated decay (NMD) of RBM3-encoding mature mRNA, or for treating or preventing a disease affected by RMB3 expression, or for providing a neuroprotective treatment, according to any one of claims 1 to 35 or 37 to 39.

42. A method of identifying an antisense oligonucleotide (ASO) capable of increasing expression of RBM3 in a cell, the method comprising
i) identifying an ASO that targets a region of the pre-mRNA of the RBM3 gene,
wherein the region is selected from: a region within exon 3a, a region spanning a splice site of exon 3a, a region located within 250 nucleotides upstream exon 3a and a region located within 250 nucleotides downstream exon 3a;
ii) delivering the ASO identified in step i) to the cell; and
iii) measuring the level of expression of RBM3 in the cell of step ii).

43. The method of claim 42, further comprising:
iv) comparing the level of expression measured in step iii) with the level of expression of RBM3 in a cell treated with a control, which is optionally an ASO or DMSO.

44. The method of claim 42 or 43, wherein the level of expression is measured by RT-qPCR or by western blotting.

45. An expression construct, which is optionally a vector, which is optionally a viral vector, encoding the ASO defined in any one of claims 19 to 24.

46. A host cell comprising the expression construct of claim 45.

47. A pair of guide RNAs for removing exon 3a from the gene of RBM3, wherein the first guide RNA is capable of hybridising to the genomic sequence upstream of exon 3a and the second guide RNA is capable of hybridising to the genomic sequence downstream of exon 3a.

48. The pair of guide RNAs of claim 47, wherein the first guide RNA comprises SEQ ID NO: 11 or SEQ ID NO: 12 and the second guide RNA comprises SEQ ID NO: 13.

49. A CRISPR/Cas-based base editing system for altering an RNA splice site encoded in the genomic DNA of a subject,
the CRISPR/Cas-based base editing system comprising a fusion protein and at least one guide RNA (gRNA),
wherein the fusion protein comprises a Cas protein and a base-editing domain,
wherein the at least one gRNA is capable of hybridising to a region in the gene of RBM3, and
wherein the region comprises:
(i) nucleotides 136 to 147, 137 to 147, 138 to 147, 139 to 147, 140 to 147, 141 to 147, 142 to 147, 143 to 147, 144 to 147, 145 to 147, or 146 to 147 of SEQ ID NO: 32, or a complement thereof, such that the splice site located at nucleotides 146-147 of SEQ ID NO: 32 is altered;
(ii) SEQ ID NO: 6, or a complement thereof, such that the splice site located at nucleotides 106-107 of SEQ ID NO: 6 is altered; or
(iii) nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33, or a complement thereof, such that the splice site located at the first two nucleotides of SEQ ID NO: 33 is altered,
optionally wherein the gRNA, or the targeting domain of the gRNA which hybridises to the target region, is 16, 17, 18, 19, 20, or 21 nucleotides long.

50. The CRISPR/Cas-based base editing system of claim 49, wherein the region comprises nucleotides 136 to 147, 137 to 147, 138 to 147, 139 to 147, 140 to 147, 141 to 147, 142 to 147, 143 to 147, 144 to 147, or 145 to 147 of SEQ ID NO: 32, or a complement thereof such that the splice site located at the last two nucleotides of SEQ ID NO: 32 is altered, and the region further comprises nucleotides 1 and 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of SEQ ID NO: 6, or a complement thereof.

51. The CRISPR/Cas-based base editing system of claim 49, wherein the region comprises nucleotides 1 to 2, 1 to 3, 1 to 4, 1 to 5, 1 to 6, 1 to 7, 1 to 8, 1 to 9, 1 to 10, 1 to 11, or 1 to 12 of SEQ ID NO: 33, or a complement thereof, such that the splice site located at the first two nucleotides of SEQ ID NO: 33 is altered, and the region further comprises nucleotides 254 to 269, 255 to 269, 256 to 269, 257 to 269, 258 to 269, 259 to 269, 260 to 269, 261 to 269, 262 to 269, 263 to 269, 264 to 269, 265 to 269, 266 to 269, or 267 to 269 of SEQ ID NO: 6.

52. The CRISPR/Cas-based base editing system of claim 49, wherein the region spans nucleotides 106-17 of SEQ ID NO: 6 and comprises 5, 6, 7, 8, 9, 10, 11, 12, 13, 14, 15, 16, 17, 18, 19, or 20 contiguous nucleotides, such that the splice site located at nucleotides 106-107 of SEQ ID NO: 6 is altered.

53. The CRISPR/Cas-based base editing system of claim 49 or claim 50, wherein the target region is selected from SEQ ID NOs: 90, 91, and 92,
and optionally the at least one gRNA comprises or consists of a sequence which is the RNA equivalent of SEQ ID NOs: 90, 91, and 92 or a complement thereof.

54. The CRISPR/Cas-based base editing system of any one of claims 49 to 53, wherein altering the RNA splice site encoded in the genomic DNA results in non-incorporation of exon 3a in the resulting mature mRNA.

55. The CRISPR/Cas-based base editing system of any one of claims 49-54, wherein the Cas protein comprises Cas9, optionally wherein the Cas protein comprises a Cas9 nickase.

56. The CRISPR/Cas-based base editing system of any one of claims 49-55, wherein the base-editing domain comprises a cytidine deaminase domain or an adenosine deaminase domain.

57. An isolated nucleic acid encoding a guide RNA capable of hybridising to a region in the gene of RBM3, wherein the isolated nucleic acid comprises a sequence selected from SEQ ID NOs: 90, 91 or 92, or a complement thereof.

58. An isolated polynucleotide encoding the CRISPR/Cas-based base editing system of any one of claims 49-56.

59. The isolated polynucleotide of claim 58, wherein the polynucleotide comprises a first polynucleotide encoding the fusion protein and a second polynucleotide encoding the at least one gRNA.

60. An expression construct, which is optionally a vector, which is optionally a viral vector, comprising the isolated polynucleotide of claim 58 or 59.

61. A cell comprising the isolated polynucleotide of claim 58 or 59, or the expression construct of claim 60.

62. A composition comprising the CRISPR/Cas-based base editing system of any one of claims 49-56, the isolated polynucleotide of claim 57-59, or the expression construct of claim 60.

63. A method of treating or preventing a disease affected by RMB3 expression in a subject, or providing neuroprotective treatment to a subject, the method comprising administering to the subject the CRISPR/Cas-based base editing system of any one of claims 49-56, the isolated nucleic acid of claim 57-59, the expression construct of claim 60, or the composition of claim 62.

64. The method of claim 63, wherein the treating or treatment is for:
a neurological disease and/or the disease is neonatal hypoxic ischemic encephalopathy, head injury, or stroke;
a neurodegenerative disease optionally selected from Alzheimer's disease, Parkinson's disease, prion disease, frontotemporal dementia, a tauopathy, amyotrophic lateral sclerosis (ALS), and vascular dementia; or
neurological damage, optionally caused during cardiac surgery or induced coma; or
depression or anxiety.


SEQUENCE LISTING (170 sequences)

SEQ ID NO: 1 - DNA, 107 bp, Homo sapiens
  source 1..107: mol_type = other DNA
tggtcgttgtcaaggaccgggagactcagcggtccaggggttttggtttcatcaccttcaccaacccagagcatgcttcagttgccatgagagccatgaacggagag

SEQ ID NO: 2 - DNA, 106 bp, Homo sapiens
  source 1..106: mol_type = other DNA
tctctggatggtcgtcagatccgtgtggatcatgcaggcaagtctgctcggggaaccagaggaggtggctttggggcccatgggcgtggtcgcagctactctagag

SEQ ID NO: 3 - DNA, 107 bp, Mus musculus
  source 1..107: mol_type = other DNA
tggttgttgtcaaggaccgggagactcaaagatcccggggttttggcttcatcaccttcacaaacccagagcatgcctcagatgcgatgagagctatgaatggagag

SEQ ID NO: 4 - DNA, 100 bp, Mus musculus
  source 1..100: mol_type = other DNA
tccctggatgggcgccaaatccgagttgatcatgcaggaaagtctgccaggggatccagagggggtgcctttggtgggcgtggtcgcagttactctagag

SEQ ID NO: 5 - DNA, 271 bp, Mus musculus
  source 1..271: mol_type = other DNA
ggggtggagggcagcggcagacagagccgggctgcctgggaggcgacgactggtcctgcatgaggcggagaagccacctgtggatggttgacctgctctgggcttaggtagtagagtctgcagagctgtgggcctggcagaggaccggagaggacctcgtgagtctgctgggatggactctgaagcctcagaccgagagaggtgtctagccctaatagtcgggcagcttagaggagtggggaggatacgtcagcctgagttcagtcaagat

SEQ ID NO: 6 - DNA, 269 bp, Homo sapiens
  source 1..269: mol_type = other DNA
ggggtggagggcagcggcagacagagcctggccgcctgggaggcgacgactggtcctgcatgaggccgagaagccacctgtggatggttgacctgccttgggcttaggtagtagagtctgcagacctgtgggcctggcccggagaggacctcgtgagtcttctgacatggactctgaagcctcaaaccaagagaggtgtctgtctagccctgatagtcgggcagcttagaggagtggggaggagaactcagcctgagttcagtcaagat

SEQ ID NO: 7 - RNA, 20 nt, Mus musculus
  source 1..20: mol_type = other RNA
tcatcaccttcaccaaccca

SEQ ID NO: 8 - RNA, 20 nt, Mus musculus
  source 1..20: mol_type = other RNA
tctagagtagctgcgaccac

SEQ ID NO: 9 - RNA, 20 nt, Mus musculus
  source 1..20: mol_type = other RNA
tcatcaccttcacaaaccca

SEQ ID NO: 10 - RNA, 20 nt, Mus musculus
  source 1..20: mol_type = other RNA
gtggtcgcagttactctaga

SEQ ID NO: 11 - RNA, 20 nt, synthetic construct
  source 1..20: mol_type = other RNA
tgtgtctgctcggggcagcg

SEQ ID NO: 12 - RNA, 19 nt, synthetic construct
  source 1..19: mol_type = other RNA
cctgtgagtgggcactgcg

SEQ ID NO: 13 - RNA, 19 nt, synthetic construct
  source 1..19: mol_type = other RNA
tcctgatgaagccattctg

SEQ ID NO: 14 - DNA, 50 bp, Mus musculus
  source 1..50: mol_type = other DNA
ctgcctgggaggcgacgactggtcctgcatgaggcggagaagccacctgt

SEQ ID NO: 15 - DNA, 50 bp, Homo sapiens
  source 1..50: mol_type = other DNA
ccgcctgggaggcgacgactggtcctgcatgaggccgagaagccacctgt

SEQ ID NO: 16 - DNA, 10 bp, synthetic construct
  source 1..10: mol_type = other DNA
ggcgacgact

SEQ ID NO: 17 - DNA, 10 bp, synthetic construct
  source 1..10: mol_type = other DNA
ggtcctgcat

SEQ ID NO: 18 - DNA, 10 bp, Mus musculus
  source 1..10: mol_type = other DNA
gaggcggaga

SEQ ID NO: 19 - DNA, 10 bp, Homo sapiens
  source 1..10: mol_type = other DNA
gaggccgaga

SEQ ID NO: 20 - DNA, 20 bp, Mus musculus
  source 1..20: mol_type = other DNA
ctgcctgggaggcgacgact

SEQ ID NO: 21 - DNA, 20 bp, Homo sapiens
  source 1..20: mol_type = other DNA
ccgcctgggaggcgacgact

SEQ ID NO: 22 - DNA, 20 bp, Mus musculus
  source 1..20: mol_type = other DNA
ggtcctgcatgaggcggaga

SEQ ID NO: 23 - DNA, 20 bp, Homo sapiens
  source 1..20: mol_type = other DNA
ggtcctgcatgaggccgaga

SEQ ID NO: 24 - DNA, 30 bp, Mus musculus
  source 1..30: mol_type = other DNA
ggcgacgactggtcctgcatgaggcggaga

SEQ ID NO: 25 - DNA, 30 bp, Homo sapiens
  source 1..30: mol_type = other DNA
ggcgacgactggtcctgcatgaggccgaga

SEQ ID NO: 26 - DNA, 50 bp, Mus musculus
  source 1..50: mol_type = other DNA
gcctggcagaggaccggagaggacctcgtgagtctgctgggatggactct

SEQ ID NO: 27 - DNA, 50 bp, Homo sapiens
  source 1..50: mol_type = other DNA
ctgtgggcctggcccggagaggacctcgtgagtcttctgacatggactct

SEQ ID NO: 28 - DNA, 20 bp, Mus musculus
  source 1..20: mol_type = other DNA
ggacctcgtgagtctgctgg

SEQ ID NO: 29 - DNA, 20 bp, Homo sapiens
  source 1..20: mol_type = other DNA
ggacctcgtgagtcttctga

SEQ ID NO: 30 - DNA, 127 bp, Mus musculus
  source 1..127: mol_type = other DNA
gtgaggcctcctgcctgcacaggggccgtctccctgtctgccactttcttcgttctttctgttttccctctatgtctgccaggggcagcgtggccactcacaggagcatgactgccttctgttctag

SEQ ID NO: 31 - DNA, 227 bp, Mus musculus
  source 1..227: mol_type = other DNA
gtaagtagtagcaggggagaccggtggattcaggtctttttgtgggattccatagctcctggtgaagccattctggtcagcggagcttggcttggacgaggcacccagaatttggtgcccagcaggaatttctgggctcttgcctacacctacctgcctgtgcccattcttccctcatccctactgtgactgacaattccttgtgtctgtctccttcccatacctag

SEQ ID NO: 32 - DNA, 147 bp, Homo sapiens
  source 1..147: mol_type = other DNA
gtggggcctcctatctgcagagggaccttgtccccatctgcggcttccttcattctttttgtttttccctctgtgtctgctcggggcagcgtggccaccaagccccgcagtgcccactcacaggagcatgactgccttttgttctag

SEQ ID NO: 33 - DNA, 230 bp, Homo sapiens
  source 1..230: mol_type = other DNA
gtaagtagtagcaaggggaacatggtgcattcaggtcattttgtgtgggattctatagctcctgatgaagccattctggggtaaaggagcttattctggaacatggcacccagaacctggtgcccagcaggaatttttggcctcttttgcccacttacccttgcctgctcttccctcaccattgccctgactgactgccaacccatcatccttgtgtctcccacacttag

SEQ ID NO: 34 - RNA, 18 nt, Mus musculus
  source 1..18: mol_type = other RNA
ctgctactacttacatct

SEQ ID NO: 35 - RNA, 18 nt, Homo sapiens
  source 1..18: mol_type = other RNA
ttgctactacttacatct

SEQ ID NO: 36 - RNA, 25 nt, Mus musculus
  source 1..25: mol_type = other RNA
gtctcccctgctactacttacatct

SEQ ID NO: 37 - RNA, 25 nt, Homo sapiens
  source 1..25: mol_type = other RNA
gttccccttgctactacttacatct

SEQ ID NO: 38 - RNA, 25 nt, Mus musculus
  source 1..25: mol_type = other RNA
ctgctactacttacatcttgactga

SEQ ID NO: 39 - RNA, 25 nt, Homo sapiens
  source 1..25: mol_type = other RNA
ttgctactacttacatcttgactga

SEQ ID NO: 40 - RNA, 19 nt, Mus musculus
  source 1..19: mol_type = other RNA
gtcgtcgcctcccaggcag

SEQ ID NO: 41 - RNA, 19 nt, Homo sapiens
  source 1..19: mol_type = other RNA
gtcgtcgcctcccaggcgg

SEQ ID NO: 42 - RNA, 19 nt, Homo sapiens
  source 1..19: mol_type = other RNA
gaccagtcgtcgcctccca

SEQ ID NO: 43 - RNA, 19 nt, Homo sapiens
  source 1..19: mol_type = other RNA
tgcaggaccagtcgtcgcc

SEQ ID NO: 44 - RNA, 19 nt, Homo sapiens
  source 1..19: mol_type = other RNA
cctcatgcaggaccagtcg

SEQ ID NO: 45 - RNA, 19 nt, Mus musculus
  source 1..19: mol_type = other RNA
tcatgcaggaccagtcgtc

SEQ ID NO: 46 - RNA, 19 nt, Homo sapiens
  source 1..19: mol_type = other RNA
tcatgcaggaccagtcgtc

SEQ ID NO: 47 - RNA, 19 nt, Mus musculus
  source 1..19: mol_type = other RNA
cgcctcatgcaggaccagt

SEQ ID NO: 48 - RNA, 19 nt, Homo sapiens
  source 1..19: mol_type = other RNA
ggcctcatgcaggaccagt

SEQ ID NO: 49 - RNA, 19 nt, Mus musculus
  source 1..19: mol_type = other RNA
ctccgcctcatgcaggacc

SEQ ID NO: 50 - RNA, 19 nt, Homo sapiens
  source 1..19: mol_type = other RNA
ctcggcctcatgcaggacc

SEQ ID NO: 51 - RNA, 19 nt, Homo sapiens
  source 1..19: mol_type = other RNA
gactcacgaggtcctctcc

SEQ ID NO: 52 - RNA, 19 nt, Mus musculus
  source 1..19: mol_type = other RNA
cagcagactcacgaggtcc

SEQ ID NO: 53 - RNA, 19 nt, Homo sapiens
  source 1..19: mol_type = other RNA
cagaagactcacgaggtcc

SEQ ID NO: 54 - RNA, 19 nt, Mus musculus
  source 1..19: mol_type = other RNA
catcccagcagactcacga

SEQ ID NO: 55 - RNA, 19 nt, Homo sapiens
  source 1..19: mol_type = other RNA
catgtcagaagactcacga

SEQ ID NO: 56 - RNA, 19 nt, Mus musculus
  source 1..19: mol_type = other RNA
gagtccatcccagcagact

SEQ ID NO: 57 - RNA, 19 nt, Homo sapiens
  source 1..19: mol_type = other RNA
gagtccatgtcagaagact

SEQ ID NO: 58 - RNA, 4298 nt, Homo sapiens
  source 1..4298: mol_type = other RNA
atcaatcgtcttccggcgcagccccgtccctgttttttgtgctcctccgagctcgctgttcgtccgggttttttacgttttaatttccaggacttgaactgccatgtcctctgaagaaggaaagctcttcgtgggagggctcaactttaacaccgacgagcaggcactggaagaccacttcagcagtttcggacctatctctgaggtggtcgttgtcaaggaccgggagactcagcggtccaggggttttggtttcatcaccttcaccaacccagagcatgcttcagttgccatgagagccatgaacggagagtctctggatggtcgtcagatccgtgtggatcatgcaggcaagtctgctcggggaaccagaggaggtggctttggggcccatgggcgtggtcgcagctactctagaggtggtggggaccagggctatgggagtggcaggtattatgacagtcgacctggagggtatggatatggatatggacgttccagagactataatggcagaaaccagggtggttatgaccgctactcaggaggaaattacagagacaattatgacaactgaaatgagacatgcacataatatagatacacaaggaataatttctgatccaggatcgtccttccaaatggctgtatttataaaggtttttggagctgcaccgaagcatcttattttatagtatatcaaccttttgtttttaaattgacctgccaaggtagctgaagaccttttagacagttccatctttttttttaaattttttctgcctatttaaagacaaattatgggacgtttgtagaacctgagtatttttctttttaccagttttttagtttgagctcttaggtttattggagctagcaataattggttctggcaagtttggccagactgacttcaaaaaattaatgtgtatccagggacattttaaaaacctgtacacagtgtttattgtggttaggaagcaatttcccaatgtacctataagaaatgtgcatcaagccagcctgaccaacatggtgaaaccccatctgtactaaacataaaaaaattagcctggcatggtggtgtacgcctgtaatcccagtgacttgggaggctgaggcaggagaatcgcttgaacccgggaggcggaggttgcagtgagctaagatcgcgccactgtactccagcctgggcaacagcgagactccatctcaaaaaaaaaggaaatgtgtatcaagaacatgattatccagcggtattttctaattcagatcatcaaactgattatatagaagagttggctttaaaatgtttgcaaatgtctttttttttttaatactggaagaaaaaatattctgttgtgtctcatacagtgcttaggatgtctttcacagagcttattaaaaagatgaaacctgagaacaaactgctttattcttactcagcccattttgcaaattaaaagtgggggcagaggtgggcggatcacctgaggtcaggagttcgagaccagcctggccaacagggcaaaaccccatctctactaaaaatacaaaagttagcggggcgtggtggcgggtgcatgtaatcccagctactcgggaggctgaggcaggagaattgcttgaacccaggaggcggaagttgcagtgagctgagattgtgccactgcactccagcctaggtgacagcaagactctgtctcaaaaaaaaaaaaatggcacatcaacgagagggaggcttggagaatatttggttggtgggtgggcagggactgccagagggttaggtgtacattgaggcctgaggcctgctggaattgggtttccttaactggtctcttatcttagtcccattgttctgagaacataagcacctaatctcatggcgtgagctaatccctatcataaatagtggtaccagttcattccttcccctgaaatgatgggggttgggcagagatgcactgaactcatctttgtgtatgagggccatgagagaggcctggtttgaaaacaatgctaagagctcagattccaggatttatgtaggggctcttctacttcccaggtatttgacctcaggtgagtcatttaatttcagccttatgccattttacattttctgtggagggttaggttaattggcatataaagccactgagaagagttggcaaagctctcagtcacggggatttgatcttttttggtgtgtgtgtgtcttgggtggtaaagtggtaattttgtaattctagctggtcttagttcctcagttctgctccctttagtcatggttctttctagtggctgtattgaggccccatgggtgttatccctccattggttctagtttggaacagaaaaatctgttgtattcatggctttcacctggctaatactgagctaagttaccaccaggttgcaaactccaggacattattgtcctgagctgcctattcccttgctggtgcgttgtggaaccctgtattattagttccagtcctggaggcctgcctcctgagtttcccagctagttgggacaggccccaatatcccttcttcctgtaacccaaacagtcatgaatttgctttggcaggtggatggagacttgggaacttccacctcacccactaagccaggcccaggctatggggcattgtggctaaccccaccaggtggatacttggtctgaggacgcatcttattctgggcctttagggagctaaggcagtgagaattggcaggaggactgctgtgaatgccttgtaggtcgggggattggagggggtcctctgctccctgtctctcaggatggcaatgtacctctcacccccagtgtcagctgaggtaggatccataatcagtaatattcctgcaacaaaatgttcctaagtggaatcaatgaagaccaaaatttatttgtataacaactctaaacctgctctgctctgctgtgtttacaatgtgctttgtgatcatccagcccagggagtcccagtctctggctgtctatctgctctaaggaagagagcccacagttctctatagtgccatagtctgtgatgaataaagttccagatttgaggtcaacccccgaccacccttaaagtgcttgttggtctcctactttggtttgtcttcagcatccaactgatgcacatttgtcaagaacccactgaggctagaaaccccccaaacactcaaggtaccttgagaacatgtatcccaagacctgcaaagaccaaacacatggcctgtaactacttttcactttcaagccctggaacttgtctgtaccaaaaatctttctgaaccattcaccaaatctcttgtcaagggccagaataaattaagagattggcttttggttttcaattaggctttcaagtcctaagcccaacccctcctatcccacctaggcagaaatcacttcagatccttgcttcctagtttaatttcttctctgcctcagttataacatccataaaatggaaataataatacccacctcacacaggggttaggagtgtttttttgttggtttttttccagacagggtctcactctgtctcccaggctggagtgcagtggcacaatctcggctcactgtaccctccgcctcccaggttcaagcagttctcatgcctcagcctcccaagtagctgggattacaggtgcctgccaccacgcccggctaatttttgtattaatagagacagggtttcaccgtgttgggcaggctggtcctgaactcctgacctcaagtgatccacccgcctcggcctcccaaagtgctgggattacaggcgtgagccaccacacctggcctaggagttaagagtattaaatgttaagaacagaccaagtccatcaaacagctccacacccttgttccatgttctggattggggagttgtgggggtggaggtgtagaacttttaaaaagcttcttaaaataagttgctgtgaatacttcaggtatataaaaaaacattggttgacaataagccagtgttctgccattcttacctgcttatcaagacaaaacctgctcaagtccctgcccagctgcattccaagtgttttcaagtttggtggtaagacctgacctgaggcttcttataacctttactcagtgggaatatgcatacagttcactacagaaatatttgtatttacttagaggaagtgccctggatctgggggcgggggggggcggggggaatgggtcttttctaaattgttaaaagcagttcatgccattattcttaataaacatttctaatatgctgtgaaa

SEQ ID NO: 59 - RNA, 1348 nt, Mus musculus
  source 1..1348: mol_type = other RNA
gctcctctcgattcccggaagcggcgaggcaaattgctgcctccgccttcctcgtcctcttcccaaccccccccccccgctggcctcaaccaatgagcacgcacgccagggcgcgctgggggacgttgtgtgacgtctcggctacgcacgctttatcaggcgtcttcccgcgccgcagtctctctgttctcccggttccttcgagctcgtcgtctctgccgtcctctgacttttaatttccaggacttgccttctgccatgtcgtctgaagaagggaaactcttcgtaggagggctcaacttcaacaccgatgaacaggcacttgaagaccacttcagcagctttgggcctatctctgaggtggttgttgtcaaggaccgggagactcaaagatcccggggttttggcttcatcaccttcacaaacccagagcatgcctcagatgcgatgagagctatgaatggagagtccctggatgggcgccaaatccgagttgatcatgcaggaaagtctgccaggggatccagagggggtgcctttggtgggcgtggtcgcagttactctagaggtggtggagaccagggatatggaagtggaagatatgacagtcgtcctggaggatatggatatgggtatgggcggtctagagactacagtggcagaagccagggtggctatgaccgctactcaggaggaaattacagagacaattatgacaactgagatggggcatgcacacaaaatgtagatacacaaggaataacacttctgatccaggatcgtccttgcaaattgctgtatttataaagatttttggagctgcgctgaaacgtctgttttagtacatcaagttctatttttgaattgagctcccaaggtagtttgttaaagaacttttagaaagctccatgtgttctttaaacattttttcccttttaaaaacaaattttaagacatttgttacagtcccaatatttttccttttatcagaatttttgtttaggcactcaggattattgtttctggccaaaagaaaaaagtggccaggctaaatcagtttttaaagttgatgtgtatttagggacattttaaaaccttgtaaacaatgtttatcatggccagaaagtgatttctgaatgtacctatgaacaatccgagtcaagatcatgattgcttagagatgttttttattaaagatcaccatcctgattatatagaagagcttctttaaaatgtttgtgaatgtcattttttaatactggaagaaaagtattctgttgtgtctgatgtgtttaggatgtcctttatggagctaattaaaagatgaaacctgaacacaaatcaatac

SEQ ID NO: 60 - RNA, 25 nt, synthetic construct
  source 1..25: mol_type = other RNA
cctccaccccctagaacagaaggca

SEQ ID NO: 61 - RNA, 20 nt, synthetic construct
  source 1..20: mol_type = other RNA
gactcactatagggagaccc

SEQ ID NO: 62 - RNA, 18 nt, synthetic construct
  source 1..18: mol_type = other RNA
tagaaggcacagtcgagg

SEQ ID NO: 63 - RNA, 20 nt, synthetic construct
  source 1..20: mol_type = other RNA
atctgcagagggaccttgtc

SEQ ID NO: 64 - RNA, 20 nt, synthetic construct
  source 1..20: mol_type = other RNA
cagacttgcctgcatgatcc

SEQ ID NO: 65 - RNA, 29 nt, Mus musculus
  source 1..29: mol_type = other RNA
aatttaagcttctgtggctgtgcctggct

SEQ ID NO: 66 - RNA, 30 nt, Homo sapiens
  source 1..30: mol_type = other RNA
aacttaagctttccggccaccctttgctac

SEQ ID NO: 67 - RNA, 34 nt, Mus musculus
  source 1..34: mol_type = other RNA
aatttctcgagttcagacataggctcttaacatt

SEQ ID NO: 68 - RNA, 31 nt, Homo sapiens
  source 1..31: mol_type = other RNA
tagactcgagataggcaactctccctctcac

SEQ ID NO: 69 - RNA, 19 nt, Mus musculus
  source 1..19: mol_type = other RNA
ccctgctactacttacatc

SEQ ID NO: 70 - RNA, 19 nt, Homo sapiens
  source 1..19: mol_type = other RNA
ccttgctactacttacatc

SEQ ID NO: 71 - skipped

SEQ ID NO: 72 - DNA, 10 bp, Mus musculus
  source 1..10: mol_type = other DNA
gcctggcaga

SEQ ID NO: 73 - DNA, 10 bp, Homo sapiens
  source 1..10: mol_type = other DNA
ctgtgggcct

SEQ ID NO: 74 - DNA, 10 bp, Mus musculus
  source 1..10: mol_type = other DNA
ggaccggaga

SEQ ID NO: 75 - DNA, 10 bp, Homo sapiens
  source 1..10: mol_type = other DNA
ggcccggaga

SEQ ID NO: 76 - DNA, 10 bp, synthetic construct
  source 1..10: mol_type = other DNA
ggacctcgtg

SEQ ID NO: 77 - DNA, 10 bp, Mus musculus
  source 1..10: mol_type = other DNA
agtctgctgg

SEQ ID NO: 78 - DNA, 10 bp, Homo sapiens
  source 1..10: mol_type = other DNA
agtcttctga

SEQ ID NO: 79 - DNA, 10 bp, Mus musculus
  source 1..10: mol_type = other DNA
gatggactct

SEQ ID NO: 80 - DNA, 10 bp, Homo sapiens
  source 1..10: mol_type = other DNA
catggactct

SEQ ID NO: 81 - DNA, 20 bp, Mus musculus
  source 1..20: mol_type = other DNA
gcctggcagaggaccggaga

SEQ ID NO: 82 - DNA, 20 bp, Homo sapiens
  source 1..20: mol_type = other DNA
ctgtgggcctggcccggaga

SEQ ID NO: 83 - RNA, 19 nt, Homo sapiens
  source 1..19: mol_type = other RNA
ctcatgcaggaccagtcgt

SEQ ID NO: 84 - RNA, 19 nt, Homo sapiens
  source 1..19: mol_type = other RNA
gcctcatgcaggaccagtc

SEQ ID NO: 85 - RNA, 19 nt, Homo sapiens
  source 1..19: mol_type = other RNA
atgcaggaccagtcgtcgc

SEQ ID NO: 86 - RNA, 19 nt, Mus musculus
  source 1..19: mol_type = other RNA
tccgcctcatgcaggacca

SEQ ID NO: 87 - RNA, 19 nt, Homo sapiens
  source 1..19: mol_type = other RNA
tcggcctcatgcaggacca

SEQ ID NO: 88 - RNA, 20 nt, synthetic construct
  source 1..20: mol_type = other RNA
ggcgacgactggtcctgcat

SEQ ID NO: 89 - RNA, 15 nt, synthetic construct
  source 1..15: mol_type = other RNA
ggacctcgtgagtct

SEQ ID NO: 90 - RNA, 20 nt, synthetic construct
  source 1..20: mol_type = other RNA
ttctagggggtggagggcag

SEQ ID NO: 91 - RNA, 20 nt, synthetic construct
  source 1..20: mol_type = other RNA
ctactacctaagcccaaggc

SEQ ID NO: 92 - RNA, 20 nt, synthetic construct
  source 1..20: mol_type = other RNA
cttacatcttgactgaactc

SEQ ID NO: 93 - RNA, 18 nt, synthetic construct
  source 1..18: mol_type = other RNA
agatgtaagtagtagcag

SEQ ID NO: 94 - RNA, 25 nt, synthetic construct
  source 1..25: mol_type = other RNA
agatgtaagtagtagcaggggagac

SEQ ID NO: 95 - RNA, 25 nt, synthetic construct
  source 1..25: mol_type = other RNA
tcagtcaagatgtaagtagtagcag

SEQ ID NO: 96 - RNA, 19 nt, synthetic construct
  source 1..19: mol_type = other RNA
gatgtaagtagtagcaggg

SEQ ID NO: 97 - RNA, 19 nt, synthetic construct
  source 1..19: mol_type = other RNA
ctgcctgggaggcgacgac

SEQ ID NO: 98 - RNA, 19 nt, Homo sapiens
  source 1..19: mol_type = other RNA
tgggaggcgacgactggtc

SEQ ID NO: 99 - RNA, 19 nt, Homo sapiens
  source 1..19: mol_type = other RNA
ggcgacgactggtcctgca

SEQ ID NO: 100 - RNA, 19 nt, Homo sapiens
  source 1..19: mol_type = other RNA
cgactggtcctgcatgagg

SEQ ID NO: 101 - RNA, 19 nt, Homo sapiens
  source 1..19: mol_type = other RNA
gacgactggtcctgcatga

SEQ ID NO: 102 - RNA, 19 nt, synthetic construct
  source 1..19: mol_type = other RNA
actggtcctgcatgaggcg

SEQ ID NO: 103 - RNA, 19 nt, synthetic construct
  source 1..19: mol_type = other RNA
ggtcctgcatgaggcggag

SEQ ID NO: 104 - RNA, 19 nt, Homo sapiens
  source 1..19: mol_type = other RNA
ggagaggacctcgtgagtc

SEQ ID NO: 105 - RNA, 19 nt, synthetic construct
  source 1..19: mol_type = other RNA
ggacctcgtgagtctgctg

SEQ ID NO: 106 - RNA, 19 nt, synthetic construct
  source 1..19: mol_type = other RNA
tcgtgagtctgctgggatg

SEQ ID NO: 107 - RNA, 19 nt, synthetic construct
  source 1..19: mol_type = other RNA
agtctgctgggatggactc

SEQ ID NO: 108 - RNA, 18 nt, Homo sapiens
  source 1..18: mol_type = other RNA
gcactgtgcaagcctctt

SEQ ID NO: 109 - RNA, 18 nt, Homo sapiens
  source 1..18: mol_type = other RNA
cgtgacacgttcggagaa

SEQ ID NO: 110 - RNA, 18 nt, Homo sapiens
  source 1..18: mol_type = other RNA
agatgtaagtagtagcaa

SEQ ID NO: 111 - RNA, 19 nt, Homo sapiens
  source 1..19: mol_type = other RNA
gatgtaagtagtagcaagg

SEQ ID NO: 112 - RNA, 19 nt, Homo sapiens
  source 1..19: mol_type = other RNA
ccgcctgggaggcgacgac

SEQ ID NO: 113 - RNA, 19 nt, Homo sapiens
  source 1..19: mol_type = other RNA
actggtcctgcatgaggcc

SEQ ID NO: 114 - RNA, 19 nt, Homo sapiens
  source 1..19: mol_type = other RNA
acgactggtcctgcatgag

SEQ ID NO: 115 - RNA, 19 nt, Homo sapiens
  source 1..19: mol_type = other RNA
gactggtcctgcatgaggc

SEQ ID NO: 116 - RNA, 19 nt, Homo sapiens
  source 1..19: mol_type = other RNA
gcgacgactggtcctgcat

SEQ ID NO: 117 - RNA, 19 nt, Homo sapiens
  source 1..19: mol_type = other RNA
tggtcctgcatgaggccga

SEQ ID NO: 118 - RNA, 19 nt, Homo sapiens
  source 1..19: mol_type = other RNA
ggtcctgcatgaggccgag

SEQ ID NO: 119 - RNA, 19 nt, Homo sapiens
  source 1..19: mol_type = other RNA
ggacctcgtgagtcttctg

SEQ ID NO: 120 - RNA, 19 nt, Homo sapiens
  source 1..19: mol_type = other RNA
tcgtgagtcttctgacatg

SEQ ID NO: 121 - RNA, 19 nt, Homo sapiens
  source 1..19: mol_type = other RNA
agtcttctgacatggactc

SEQ ID NO: 122 - RNA, 18 nt, Mus musculus
  source 1..18: mol_type = other RNA
aagggaaactcttcgtag

SEQ ID NO: 123 - RNA, 23 nt, Mus musculus
  source 1..23: mol_type = other RNA
gacaacaaccacctcagagatag

SEQ ID NO: 124 - RNA, 29 nt, Mus musculus
  source 1..29: mol_type = other RNA
caacgggggacataaaagttattggtgga

SEQ ID NO: 125 - RNA, 26 nt, Mus musculus
  source 1..26: mol_type = other RNA
tgcaaccttaaccattttggggctgt

SEQ ID NO: 126 - RNA, 20 nt, Homo sapiens
  source 1..20: mol_type = other RNA
aaggaaagctcttcgtggga

SEQ ID NO: 127 - RNA, 20 nt, Homo sapiens
  source 1..20: mol_type = other RNA
gacaacgaccacctcagaga

SEQ ID NO: 128 - RNA, 25 nt, Homo sapiens
  source 1..25: mol_type = other RNA
cttcgctctctgctcctcctgttcg

SEQ ID NO: 129 - RNA, 24 nt, Homo sapiens
  source 1..24: mol_type = other RNA
accaggcgcccaatacgaccaaat

SEQ ID NO: 130 - RNA, 646 nt, synthetic construct
  source 1..646: mol_type = other RNA
gtggggcctcctatctgcagagggaccttgtccccatctgcggcttccttcattctttttgtttttccctctgtgtctgctcggggcagcgtggccaccaagccccgcagtgcccactcacaggagcatgactgccttttgttctagggggtggagggcagcggcagacagagcctggccgcctgggaggcgacgactggtcctgcatgaggccgagaagccacctgtggatggttgacctgccttgggcttaggtagtagagtctgcagacctgtgggcctggcccggagaggacctcgtgagtcttctgacatggactctgaagcctcaaaccaagagaggtgtctgtctagccctgatagtcgggcagcttagaggagtggggaggagaactcagcctgagttcagtcaagatgtaagtagtagcaaggggaacatggtgcattcaggtcattttgtgtgggattctatagctcctgatgaagccattctggggtaaaggagcttattctggaacatggcacccagaacctggtgcccagcaggaatttttggcctcttttgcccacttacccttgcctgctcttccctcaccattgccctgactgactgccaacccatcatccttgtgtctcccacacttag

SEQ ID NO: 131 - DNA, 32 bp, synthetic construct
  source 1..32: mol_type = other DNA
tcagtcaagatgtaagtagtagcaggggagac

SEQ ID NO: 132 - DNA, 39 bp, synthetic construct
  source 1..39: mol_type = other DNA
ctgcctgggaggcgacgactggtcctgcatgaggcggag

SEQ ID NO: 133 - RNA, 19 nt, synthetic construct
  source 1..19: mol_type = other RNA
tcatgcaggaccagtcgct

SEQ ID NO: 134 - DNA, 34 bp, synthetic construct
  source 1..34: mol_type = other DNA
ggagaggacctcgtgagtctgctgggatggactc

SEQ ID NO: 135 - RNA, 18 nt, synthetic construct
  source 1..18: mol_type = other RNA
  modified_base 1..18: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  misc_feature 1..18: note = MOE PS-MOE
ctgctactacttacatct

SEQ ID NO: 136 - RNA, 25 nt, synthetic construct
  source 1..25: mol_type = other RNA
  modified_base 1..25: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
  modified_base 19^20: mod_base = OTHER; note = phosphorothioate linkage
  modified_base 20^21: mod_base = OTHER; note = phosphorothioate linkage
  modified_base 21^22: mod_base = OTHER; note = phosphorothioate linkage
  modified_base 22^23: mod_base = OTHER; note = phosphorothioate linkage
  modified_base 23^24: mod_base = OTHER; note = phosphorothioate linkage
  modified_base 24^25: mod_base = OTHER; note = phosphorothioate linkage
  misc_feature 1..25: note = MOElongA PS-MOE
gtctcccctgctactacttacatct

SEQ ID NO: 137 - RNA, 25 nt, synthetic construct
  source 1..25: mol_type = other RNA
  modified_base 1..25: mod_base = OTHER; note = 2-prime-O-methoxyethyl nucleotides
  modified_base 1^2: mod_base = OTHER; note = phosphorothioate linkage
  misc_feature 1..25: note = MOElongB PS-MOE
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
  modified_base 19^20: mod_base = OTHER; note = phosphorothioate linkage
  modified_base 20^21: mod_base = OTHER; note = phosphorothioate linkage
  modified_base 21^22: mod_base = OTHER; note = phosphorothioate linkage
  modified_base 22^23: mod_base = OTHER; note = phosphorothioate linkage
  modified_base 23^24: mod_base = OTHER; note = phosphorothioate linkage
  modified_base 24^25: mod_base = OTHER; note = phosphorothioate linkage
ctgctactacttacatcttgactga

SEQ ID NO: 138 - RNA, 19 nt, synthetic construct
  source 1..19: mol_type = other RNA
  misc_feature 1..19: note = MOE’ PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ccctgctactacttacatc

SEQ ID NO: 139 - RNA, 19 nt, synthetic construct
  source 1..19: mol_type = other RNA
  misc_feature 1..19: note = M2A PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gtcgtcgcctcccaggcag

SEQ ID NO: 140 - RNA, 19 nt, synthetic construct
  source 1..19: mol_type = other RNA
  misc_feature 1..19: note = M2B PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gaccagtcgtcgcctccca

SEQ ID NO: 141 - RNA, 19 nt, synthetic construct
  source 1..19: mol_type = other RNA
  misc_feature 1..19: note = M2C PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
tgcaggaccagtcgtcgcc

SEQ ID NO: 142 - RNA, 19 nt, synthetic construct
  source 1..19: mol_type = other RNA
  misc_feature 1..19: note = M2D PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
cctcatgcaggaccagtcg

SEQ ID NO: 143 - RNA, 19 nt, synthetic construct
  source 1..19: mol_type = other RNA
  misc_feature 1..19: note = M2Da PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
tcatgcaggaccagtcgtc

SEQ ID NO: 144 - RNA, 19 nt, synthetic construct
  source 1..19: mol_type = other RNA
  misc_feature 1..19: note = M2Db PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
cgcctcatgcaggaccagt

SEQ ID NO: 145 - RNA, 19 nt, synthetic construct
  source 1..19: mol_type = other RNA
  misc_feature 1..19: note = M2E PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ctccgcctcatgcaggacc

SEQ ID NO: 146 - RNA, 19 nt, synthetic construct
  source 1..19: mol_type = other RNA
  misc_feature 1..19: note = M4A PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gactcacgaggtcctctcc

SEQ ID NO: 147 - RNA, 19 nt, synthetic construct
  source 1..19: mol_type = other RNA
  misc_feature 1..19: note = M4B PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
cagcagactcacgaggtcc

SEQ ID NO: 148 - RNA, 19 nt, synthetic construct
  source 1..19: mol_type = other RNA
  misc_feature 1..19: note = M4C PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
catcccagcagactcacga

SEQ ID NO: 149 - RNA, 19 nt, synthetic construct
  source 1..19: mol_type = other RNA
  misc_feature 1..19: note = M4D PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gagtccatcccagcagact

SEQ ID NO: 150 - RNA, 18 nt, synthetic construct
  source 1..18: mol_type = other RNA
  misc_feature 1..18: note = CTRL PS-MOE
  modified_base 1..18: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
ttctccgaacgtgtcacg

SEQ ID NO: 151 - RNA, 18 nt, synthetic construct
  source 1..18: mol_type = other RNA
  misc_feature 1..18: note = hMOE PS-MOE
  modified_base 1..18: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
ttgctactacttacatct

SEQ ID NO: 152 - RNA, 19 nt, synthetic construct
  source 1..19: mol_type = other RNA
  misc_feature 1..19: note = hMOE’PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ccttgctactacttacatc

SEQ ID NO: 153 - RNA, 19 nt, synthetic construct
  source 1..19: mol_type = other RNA
  misc_feature 1..19: note = hM2A PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gtcgtcgcctcccaggcgg

SEQ ID NO: 154 - RNA, 19 nt, synthetic construct
  source 1..19: mol_type = other RNA
  misc_feature 1..19: note = hM2B PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gaccagtcgtcgcctccca

SEQ ID NO: 155 - RNA, 19 nt, synthetic construct
  source 1..19: mol_type = other RNA
  misc_feature 1..19: note = hM2C PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
tgcaggaccagtcgtcgcc

SEQ ID NO: 156 - RNA, 19 nt, synthetic construct
  source 1..19: mol_type = other RNA
  misc_feature 1..19: note = hM2D PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
cctcatgcaggaccagtcg

SEQ ID NO: 157 - RNA, 19 nt, synthetic construct
  source 1..19: mol_type = other RNA
  misc_feature 1..19: note = hM2Da PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
tcatgcaggaccagtcgtc

SEQ ID NO: 158 - RNA, 19 nt, synthetic construct
  source 1..19: mol_type = other RNA
  misc_feature 1..19: note = hM2Db PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ggcctcatgcaggaccagt

SEQ ID NO: 159 - RNA, 19 nt, synthetic construct
  source 1..19: mol_type = other RNA
  misc_feature 1..19: note = hM2Dc PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ctcatgcaggaccagtcgt

SEQ ID NO: 160 - RNA, 19 nt, synthetic construct
  source 1..19: mol_type = other RNA
  misc_feature 1..19: note = hM2Dd PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gcctcatgcaggaccagtc

SEQ ID NO: 161 - RNA, 19 nt, synthetic construct
  source 1..19: mol_type = other RNA
  misc_feature 1..19: note = hM2De PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
atgcaggaccagtcgtcgc

SEQ ID NO: 162 - RNA, 19 nt, synthetic construct
  source 1..19: mol_type = other RNA
  misc_feature 1..19: note = hM2Df PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
tcggcctcatgcaggacca

SEQ ID NO: 163 - RNA, 19 nt, synthetic construct
  source 1..19: mol_type = other RNA
  misc_feature 1..19: note = hM2E PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
ctcggcctcatgcaggacc

SEQ ID NO: 164 - RNA, 19 nt, synthetic construct
  source 1..19: mol_type = other RNA
  misc_feature 1..19: note = hM4A PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gactcacgaggtcctctcc

SEQ ID NO: 165 - RNA, 19 nt, synthetic construct
  source 1..19: mol_type = other RNA
  misc_feature 1..19: note = hM4B PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
cagaagactcacgaggtcc

SEQ ID NO: 166 - RNA, 19 nt, synthetic construct
  source 1..19: mol_type = other RNA
  misc_feature 1..19: note = hM4C PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
catgtcagaagactcacga

SEQ ID NO: 167 - RNA, 19 nt, synthetic construct
  source 1..19: mol_type = other RNA
  misc_feature 1..19: note = hM4D PS-MOE
  modified_base 1..19: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
  modified_base 18^19: mod_base = OTHER; note = phosphorothioate linkage
gagtccatgtcagaagact

SEQ ID NO: 168 - RNA, 18 nt, synthetic construct
  source 1..18: mol_type = other RNA
  misc_feature 1..18: note = CTRL PS-MOE
  modified_base 1..18: mod_base = OTHER; note = 2-prime-O-methoxyethyl nucleotides
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 16^17: mod_base = OTHER; note = phosphorothioate linkage
  modified_base 17^18: mod_base = OTHER; note = phosphorothioate linkage
ttctccgaacgtgtcacg

SEQ ID NO: 169 - RNA, 25 nt, synthetic construct
  source 1..25: mol_type = other RNA
  modified_base 1..25: mod_base = OTHER; note = Morpholino oligonucleotides
  modified_base 1^2: mod_base = OTHER; note = Phosphorodiamidate linkage
  modified_base 2^3: mod_base = OTHER; note = Phosphorodiamidate linkage
  modified_base 3^4: mod_base = OTHER; note = Phosphorodiamidate linkage
  modified_base 4^5: mod_base = OTHER; note = Phosphorodiamidate linkage
  modified_base 5^6: mod_base = OTHER; note = Phosphorodiamidate linkage
  modified_base 6^7: mod_base = OTHER; note = Phosphorodiamidate linkage
  modified_base 7^8: mod_base = OTHER; note = Phosphorodiamidate linkage
  modified_base 8^9: mod_base = OTHER; note = Phosphorodiamidate linkage
  modified_base 9^10: mod_base = OTHER; note = Phosphorodiamidate linkage
  modified_base 10^11: mod_base = OTHER; note = Phosphorodiamidate linkage
  modified_base 11^12: mod_base = OTHER; note = Phosphorodiamidate linkage
  modified_base 12^13: mod_base = OTHER; note = Phosphorodiamidate linkage
  modified_base 13^14: mod_base = OTHER; note = Phosphorodiamidate linkage
  modified_base 14^15: mod_base = OTHER; note = Phosphorodiamidate linkage
  modified_base 15^16: mod_base = OTHER; note = Phosphorodiamidate linkage
  modified_base 16^17: mod_base = OTHER; note = Phosphorodiamidate linkage
  modified_base 17^18: mod_base = OTHER; note = Phosphorodiamidate linkage
  modified_base 18^19: mod_base = OTHER; note = Phosphorodiamidate linkage
  modified_base 19^20: mod_base = OTHER; note = Phosphorodiamidate linkage
  modified_base 20^21: mod_base = OTHER; note = Phosphorodiamidate linkage
  modified_base 21^22: mod_base = OTHER; note = Phosphorodiamidate linkage
  modified_base 22^23: mod_base = OTHER; note = Phosphorodiamidate linkage
  modified_base 23^24: mod_base = OTHER; note = Phosphorodiamidate linkage
  modified_base 24^25: mod_base = OTHER; note = Phosphorodiamidate linkage
  misc_feature 1..25: note = 5-prime ss morpholino
gtctcccctgctactacttacatct

SEQ ID NO: 170 - RNA, 25 nt, synthetic construct
  source 1..25: mol_type = other RNA
  modified_base 1..25: mod_base = OTHER; note = Morpholino oligonucleotides
  modified_base 1^2: mod_base = OTHER; note = Phosphorodiamidate linkage
  modified_base 2^3: mod_base = OTHER; note = Phosphorodiamidate linkage
  modified_base 3^4: mod_base = OTHER; note = Phosphorodiamidate linkage
  modified_base 4^5: mod_base = OTHER; note = Phosphorodiamidate linkage
  modified_base 5^6: mod_base = OTHER; note = Phosphorodiamidate linkage
  modified_base 6^7: mod_base = OTHER; note = Phosphorodiamidate linkage
  modified_base 7^8: mod_base = OTHER; note = Phosphorodiamidate linkage
  modified_base 8^9: mod_base = OTHER; note = Phosphorodiamidate linkage
  modified_base 9^10: mod_base = OTHER; note = Phosphorodiamidate linkage
  modified_base 10^11: mod_base = OTHER; note = Phosphorodiamidate linkage
  modified_base 11^12: mod_base = OTHER; note = Phosphorodiamidate linkage
  modified_base 12^13: mod_base = OTHER; note = Phosphorodiamidate linkage
  modified_base 13^14: mod_base = OTHER; note = Phosphorodiamidate linkage
  modified_base 14^15: mod_base = OTHER; note = Phosphorodiamidate linkage
  modified_base 15^16: mod_base = OTHER; note = Phosphorodiamidate linkage
  modified_base 16^17: mod_base = OTHER; note = Phosphorodiamidate linkage
  modified_base 17^18: mod_base = OTHER; note = Phosphorodiamidate linkage
  modified_base 18^19: mod_base = OTHER; note = Phosphorodiamidate linkage
  modified_base 19^20: mod_base = OTHER; note = Phosphorodiamidate linkage
  modified_base 20^21: mod_base = OTHER; note = Phosphorodiamidate linkage
  modified_base 21^22: mod_base = OTHER; note = Phosphorodiamidate linkage
  modified_base 22^23: mod_base = OTHER; note = Phosphorodiamidate linkage
  modified_base 23^24: mod_base = OTHER; note = Phosphorodiamidate linkage
  modified_base 24^25: mod_base = OTHER; note = Phosphorodiamidate linkage
  misc_feature 1..25: note = 3-prime ss morpholino
cctccaccccctagaacagaaggca